(19) United States
(12) Patent Application Publication
(10) Pub. No.: US 2019/0194150 A1
(43) Pub. Date: Jun. 27, 2019

(54) COMPOUNDS AND METHODS FOR MODULATING RNA FUNCTION

(71) Applicant: Arrakis Therapeutics, Inc., Waltham, MA (US)

(72) Inventors: Russell C. Petter, Stow, MA (US); James Gregory Barsoum, Lexington, MA (US); Gnanasambandam Kumaravel, Lexington, MA (US); Kenneth W. Bair, Wellesley, MA (US)

(73) Assignee: Arrakis Therapeutics, Inc., Waltham, MA (US)

(21) Appl. No.: 16/314,127

(22) PCT Filed: Jun. 30, 2017
(86) PCT No.: PCT/US2017/040514

§ 371 Date: Dec. 28, 2018

Related U.S. Application Data
(60) Provisional application No. 62/357,654, filed on Jul. 1, 2016.
(60) Provisional application No. 62/453,487, filed on Feb. 1, 2017.

(51) Int. Cl. C07D 263/24; C07D 413/04; C07C 237/26; C07H 5/06; C07D 487/04

(52) U.S. Cl. CPC C07D 263/24; C07D 413/04; C07D 487/04; C07H 5/06; C07C 237/26

(57) ABSTRACT

The present invention provides compounds, compositions thereof, and methods of using the same.

### TECHNICAL FIELD OF THE INVENTION

The present invention relates to compounds and methods of use thereof for modulating the biology of RNA transcripts to treat various diseases and conditions by the binding of such compounds to trans or cis three-way junctions. The invention also provides methods of identifying RNA transcripts that can form new three-way junctions stabilized by small molecule binders, thus rendering such three-way junctions druggable, and designing and screening drug candidates that will bind to RNA three-way junctions.

### BACKGROUND OF THE INVENTION

Mammalian diseases are ultimately mediated by the transcriptome, while viruses and other pathogens depend on RNA for various aspects of infection, reproduction, and survival. Insofar as messenger mRNA (mRNA) is part of the transcriptome, and all protein expression derives from mRNAs, there is the potential to intervene in protein-mediated diseases by modulating the expression of the relevant protein and by, in turn, modulating the translation of the corresponding upstream mRNA. But mRNA is only a small portion of the transcriptome: other transcribed RNAs also regulate cellular biology either directly by the structure and function of RNA structures (e.g., ribonucleoproteins) as well as via protein expression and action, including (but not limited to) miRNA, lncRNA, lincRNA, snoRNA, snRNA, scaRNA, piRNA, ceRNA, and pseudo-genes. RNA folds into secondary and tertiary structures in which portions of the nucleic acid form double-stranded segments through Watson-Crick base pairing while other segments remain unpaired (single-stranded). One common structure is a three-way junction (3WJ). In this instance, the nucleic acid forms three double helices in a Y-shaped pattern, with the central point of intersection of the three helices termed the 3WJ. Small molecules can bind with high affinity to the 3WJ and stabilize this structure, thus affecting the function of the target RNA by altering its structural preferences and hence modulating its downstream biological activity. However, the discovery and use of small molecules as ligands for RNA to treat RNA-mediated diseases has received little attention from the pharmaceutical industry.

Accordingly, targeting the RNA transcriptome and 3WJ in particular with small molecules represents an untapped therapeutic approach to treat RNA-mediated diseases. Furthermore, there remains a high, unmet need for improved treatments of RNA-mediated diseases, including mammalian RNA-mediated diseases, viruses, parasites, and microbes.

### SUMMARY OF THE INVENTION

The present invention provides compounds and methods of use thereof for the modulation of the levels and/or activities of nucleic acid, e.g. RNA or DNA, molecules by drug-like small molecules. The present invention also provides methods of screening nucleic acid sequences for cis or trans sequence complementarity likely to form 3WJ or likely to form 3WJ in the presence of a disclosed compound. Furthermore, the present invention provides methods of screening small molecules for binding to 3WJ. The present invention takes advantage of 3WJ formation by identifying these structures and screening for small molecules that selectively bind them to stabilize the 3WJ in cells and animals. By stabilizing the 3WJ, the small molecule can modulate the stability or activity of the nucleic acid.

### BRIEF DESCRIPTION OF THE FIGURES

FIG. 1A shows a fully complementary interaction between an miRNA and mRNA (i.e. the sequences share full sequence complementarity and form a RNA-RNA duplex). FIG. 1B shows an interaction in which the miRNA is splinted discontinuously across the flank region of the mRNA located at the base of the stem-loop element. This interaction creates a 3WJ at the base of the stem-loop element.

FIG. 2 shows a flowchart linking a hypothetical target RNA of interest to an effector RNA to 3WJ-binding small molecules to molecular mechanism in cells. Importantly, using the disclosed methods it is possible to begin with either a known RNA target and identify effector/target pairs that form 3WJs using a bioinformatic screen, or perform a bioinformatic screen of the entire transcriptome to identify all effector/target pairs that form 3WJs, followed by selection of an effector/target RNA pair of interest. Following the remaining steps of the flowchart identifies small molecules that bind 3WJs on targets of high therapeutic value, thereby modulating the target to treat or ameliorate a disease or disorder.

FIG. 3 shows exemplary effector RNA/target RNA pairings. For example, miRNA and mRNA could form a 3WJ in trans, priRNA could form a cis 3WJ, and so on.

FIG. 4 shows the mechanism of 3WJ binding a small molecule (represented by a triangle) with a stem-loop (3WJ) in the effector RNA. The RNA may or may not be bound to an RNA binding protein (RBP; represented by oval). Binding of the small molecule may (or may not) influence binding of any RBP that normally binds to the RNA/RNA duplex.

FIG. 5 shows the mechanism of 3WJ binding a small molecule with a stem-loop (3WJ) in the target nucleic acid. The RNA may or may not be bound to an RNA binding protein (RBP; represented by oval). Binding of the small molecule may (or may not) influence binding of any RBP that normally binds to the RNA/RNA duplex.

FIG. 6 shows two strategies for targeting trans-acting 3-way junctions. In one strategy (top), first one identifies a “natural” miRNA/mRNA pair that yields a 3WJ; then identifies small molecule ligands that lock in the miRNA/mRNA trans-regulatory structure to amplify the miRNA regulatory effect. Notably, the small molecule can thus act as either a translational agonist (amplify translation & protein expression) or a translational antagonist (suppress translation & protein expression). In a second strategy (bottom), first one identifies “candidate” miRNA/mRNA pairs that will present a 3WJ; then identifies SM ligands that lock in miRNA/mRNA trans-regulatory structure to amplify the miRNA regulatory effect. Notably, the small molecule can thus act as either translational agonist (amplify translation & protein expression) or a translational antagonist (suppress translation & protein expression).

FIG. 7 shows a scheme demonstrating how small molecules could act to stabilize trans-acting 3WJs. Using the disclosed methods, for example one could correlate ca. 1000 miRNAs with 20,000 mRNAs for 20,000,000 potential pairs—optionally with the simplifying assumption that each mRNA has only one hybridizable site. This catalog can be assembled without reference to the actual endogenous regulatory role of a given miRNA. Insofar as the small molecule can stabilize the ternary complex, this may render moot the contributions of Argonaute and/or RBPs to regulation of mRNA function.

FIG. 8 shows exemplary binding modes of 3WJ-binding small molecules. Small molecules might bind the 3WJ only, the 3WJ plus binding the major or minor groove adjacent to one base pair, the 3WJ plus binding the major or minor groove adjacent to one base pair in two separate duplexes, and so on.

FIG. 9 shows the topology of 3WJs. Note: (A) X, Y, and Z vary independently as 0, 1, 2, 3, 4, 5, or 6. (B) Nucleotides in the loops that define the periphery of the 3WJ vary independently as A, C, G, or U. (C) Inside the cells those nucleotides may have undergone post-transcriptional modifications. (D) The stems are minimally 4 base pairs each, but can contain bulges and loops. (E) The loop at the end of the stem-loop can vary from 3 on up.

FIG. 10 shows a cis model system that may be used to understand a corresponding trans 3WJ system. Synthetic single-stranded RNA can recapitulate key binding events. It can link via 5′-to-3′ or 3′-to-5′. The length and structure of the linker will be selected to favor but not enforce 3WJ formation. Ligands that bind to 3WJs in the cis model must induce formation of ternary complex in the trans model. This format enables rapid and combinatoric selectivity screening. The binding free energy here is expected to be predictive of cellular outcome. Some features of this method: the structure to be analyzed is small—small enough to screen by NMR; easy to make; screenable via DEL (DNA encoded libraries); biologically relevant—models the regulatory interaction of miRNA with its cognate mRNA; biologically specific—focus on single mRNA; immediate focus on one subsite, so one can design structure-informed displacement assays; inherent connection between target/subsite and biological function; and the potential for co-opting non-cognate miRNAs to tackle arbitrary mRNAs at arbitrary sites.

FIG. 11 shows how the PEARL-seq method (Hook the Worm, or HTW; see U.S. Ser. No. 62/62/289,671, which is hereby incorporated by reference) can reveal endogenous mRNA/miRNA pairs. WH=warhead moiety that reacts with proximate 2′-OH sites on an RNA after binding of a small molecule ligand that is covalently attached to the WH. Separate sequencing of RNA/RNA pairs such as miRNA/mRNA reveals which nucleotides are proximate to each other and to the binding site of the HTW molecule under the assay conditions and/or in vivo.

FIG. 12 shows exemplary triptycene scaffolds with 1 auxiliary (i.e. group capable of binding to a nucleic acid feature, such as the major or minor groove, outside of the central cavity of the 3WJ).

FIG. 13 shows exemplary triptycene scaffolds with 2 auxiliaries.

FIG. 14 shows exemplary triptycene scaffolds with 3 auxiliaries.

FIG. 15 shows exemplary trityl scaffolds with 1 auxiliary.

FIG. 16 shows exemplary trityl scaffolds with 2 auxiliaries.

FIG. 17 shows exemplary 1-azabicyclooctane scaffolds with 1 auxiliary.

FIG. 18 shows exemplary 1-azabicyclooctane scaffolds with 2 auxiliaries.

FIG. 19 shows exemplary trioxabicyclooctane scaffold with 1 auxiliary.

FIG. 20 shows exemplary trioxabicyclooctane scaffolds with 2 auxiliaries.

FIG. 21 shows exemplary adamantane scaffolds with 2 auxiliaries.

FIG. 22 shows exemplary adamantane scaffolds with 2 auxiliaries.

FIG. 23 shows general molecular scaffolds that may bind the central cavity of 3WJs and also bind to double stranded RNA grooves by interacting with exposed edges of base pairs when substituted with one or more functional groups or edge binders as described in various embodiments herein.

FIG. 24 shows structures of DNA groove binders that provide a variety of synthons for edge interactions.

FIG. 25 shows structures of exemplary small molecule scaffolds that feature functional groups capable of base-pairing with nucleic acid target 3WJs, as well as functional groups capable of binding interactions with edge features such as the minor groove.

FIG. 26 shows pictorial representations and an exemplary compound structure of a designed small molecule that features a nucleobase capable of base-pairing with nucleic acid target 3WJs, as well as functional groups capable of binding interactions with edge features such as the major or minor groove. H-bond donor and acceptor functionalities are spaced to optimize interactions (including sequence-specific interactions) with the stem of a nucleic acid stem-loop structure.

FIG. 27 shows exemplary binding modes for designed small molecules optimized for geometries of given nucleic acid junction structures. Binding focuses not only on the central cavity but also includes “arms” that bind with stem structures away from the central cavity, optionally including functionality capable of binding in distant secondary loops.

FIG. 28 shows exemplary binding modes for designed small molecules optimized for geometries of given nucleic acid duplexes that have one or more bulges (unpaired nucleotide(s)). Here, hydrogen bond donors and acceptors are spaced to interact with the duplex, such as in the major or minor groove, with one or more nucleobases placed to interact with the bulge(s).

FIG. 29 shows exemplary binding modes for designed small molecules optimized for geometries of given nucleic acid duplexes that have one or more bulges (unpaired nucleotide(s)). Here, hydrogen bond donors and acceptors are spaced to interact with the duplex, such as in the major or minor groove, with one or more nucleobases placed to interact with the bulge(s).

FIG. 30 shows exemplary binding modes for designed small molecules optimized for geometries of given nucleic acid 3WJs. Here, hydrogen bond donors and acceptors are spaced to interact with one or more duplex arms protruding from the 3WJ, such as interacting with the major or minor groove, with one or more nucleobases placed to interact with distant loops and a nucleobase (left structure) or chemical scaffold such as a compound of Formula I, II, etc. optionally placed for binding in the central cavity of the 3WJ (three rightmost structures).

FIG. 31 shows a list of exemplary target mRNAs that, in certain embodiments, are down-regulated by a disclosed compound.

FIG. 32 shows a list of exemplary target mRNAs that, in certain embodiments, are up-regulated by a disclosed compound.

FIG. 33 shows structures of exemplary DNA groove binders that provide a variety of synthons for edge interactions.

FIGS. 34-67 show exemplary compound scaffolds, small molecules, and synthetic methods of preparing same, for use in the present invention.

### DETAILED DESCRIPTION OF THE INVENTION

The present invention provides compounds and methods of use thereof for the modulation of the levels and/or activities of nucleic acid, e.g. RNA or DNA, molecules by drug-like small molecules. The present invention also provides methods of screening nucleic acid sequences for cis or trans sequence complementarity likely to form 3WJ or likely to form 3WJ in the presence of a disclosed compound. Furthermore, the present invention provides methods of screening small molecules for binding to 3WJ. The present invention takes advantage of 3WJ formation by identifying these structures and screening for small molecules that selectively bind them to stabilize the 3WJ in cells and animals. By stabilizing the 3WJ, the small molecule can modulate the stability or activity of the nucleic acid.

In one aspect, the present invention provides a compound or pharmaceutically acceptable salt thereof that selectively binds to and stabilizes a 3WJ. In some embodiments, the 3WJ is the product of cis interactions, i.e. results from interactions within the same nucleic acid, such as RNA, sequence or strand. In some embodiments, the 3WJ is the product of trans interactions i.e. results from interactions within two or more nucleic acid, such as RNA-RNA, RNA-DNA, or DNA-DNA, sequences or strands. Thus, in a cis 3WJ, the sequences comprising the 3WJ are all within one nucleic acid molecule. In contrast, in a trans 3WJ, the sequences forming the 3WJ are derived from the interaction of two or three, most commonly two, different nucleic acid molecules. For example, in some cis 3WJ, sequences in the 5′ untranslated region (UTR) of a single mRNA transcript fold to form a 3WJ. In other embodiments, an exemplary trans 3WJ is a 3WJ formed between a microRNA (miRNA) and an mRNA.

A variety of RNAs could be targeted for 3WJ formation and modulation by a small molecule that binds to the 3WJ. These include mRNA, long noncoding RNA (lncRNA), viral RNA, and microbial RNA. These will be referred to in this patent application as “targets”. One common use for this invention will be to target human mRNA in order to affect the level of the protein expressed by translation of that mRNA. Another use will be to target the genomes of RNA viruses to inhibit translation, block viral packaging, or inhibit other obligatory steps in the virus life cycle.

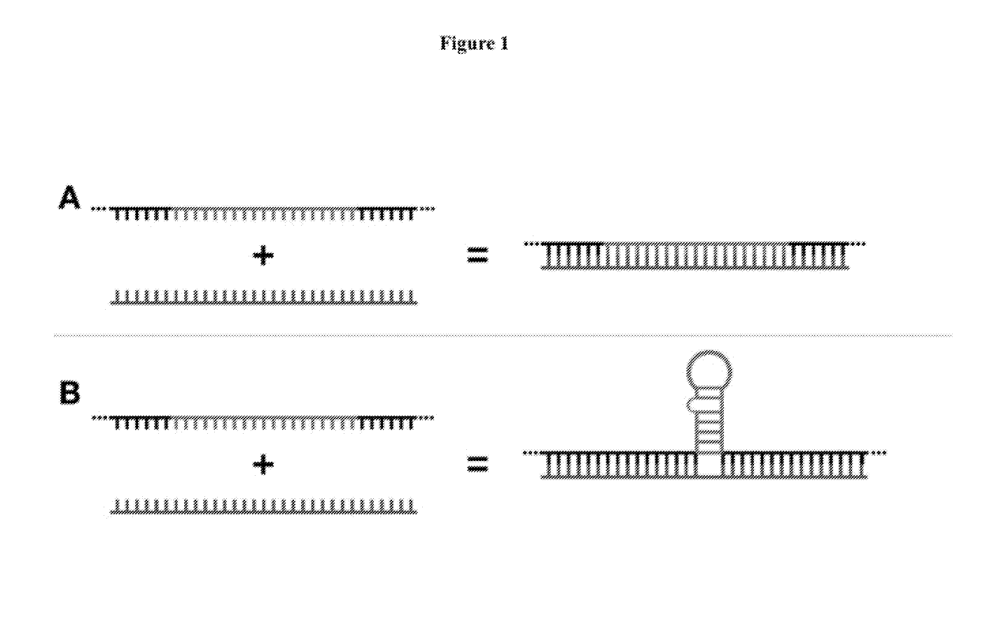

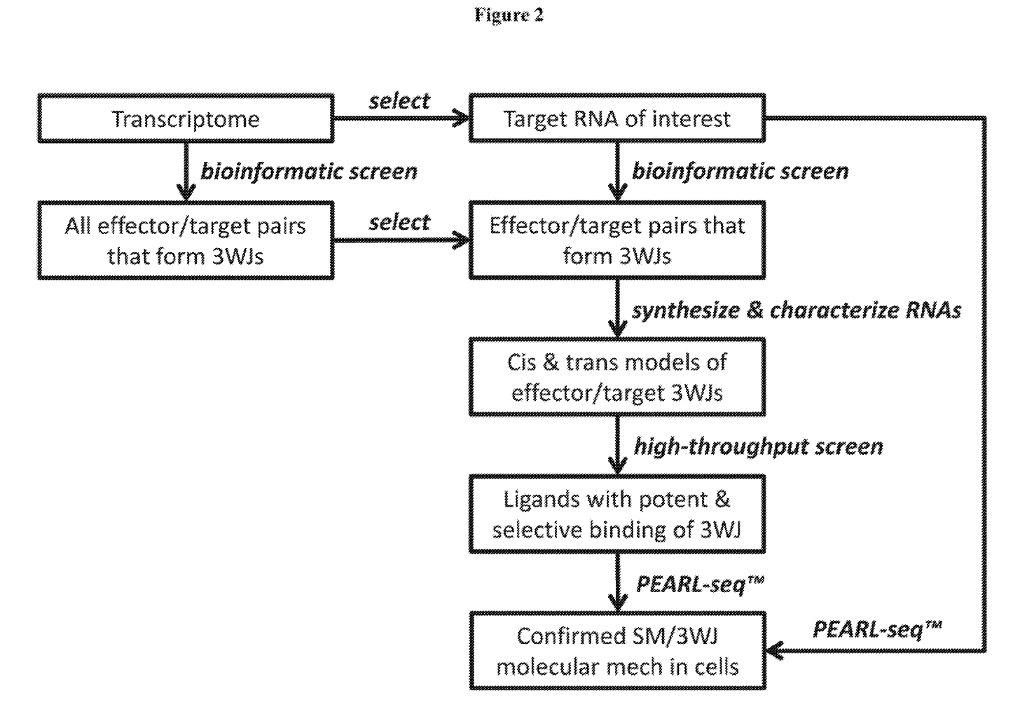

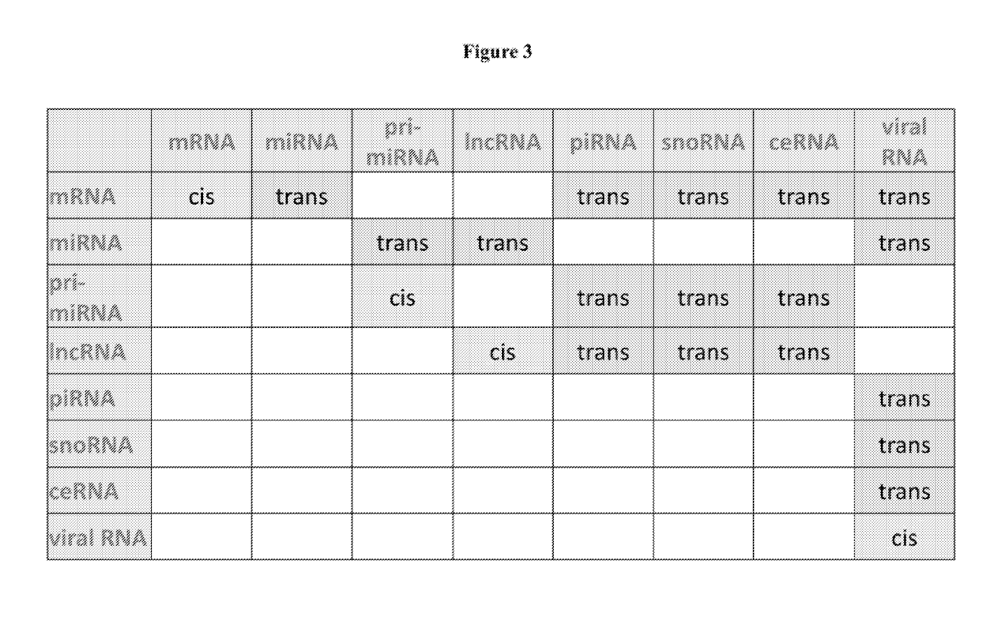

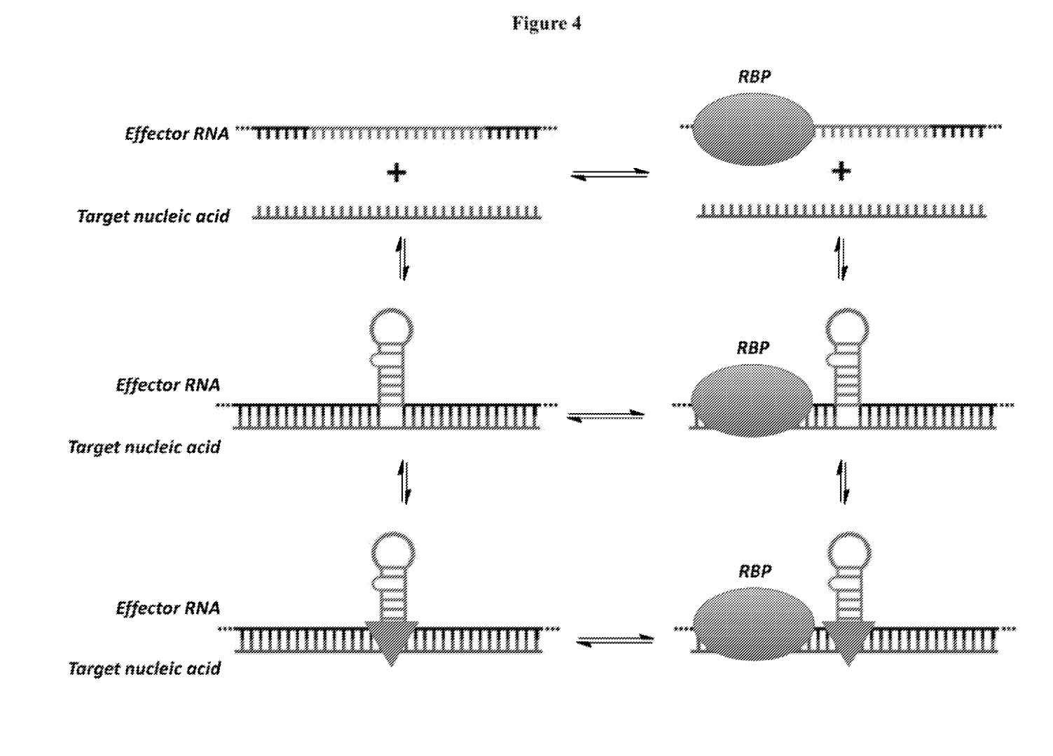

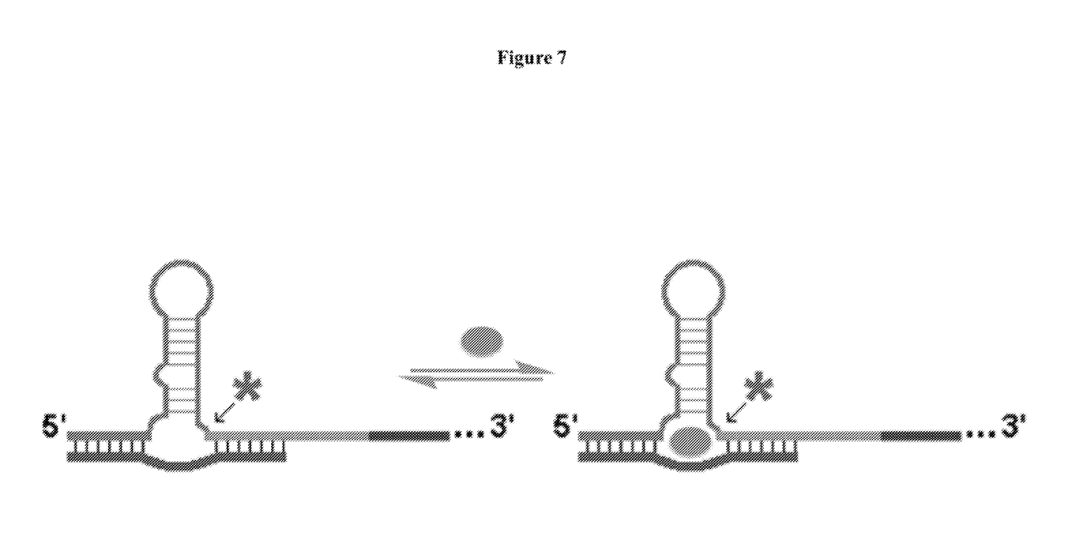

Small RNAs can act in trans to induce 3WJ formation. These RNAs will be referred to as “effectors”. In some embodiments, effectors are selected from various natural forms of small RNA such as miRNA, Piwi-interacting RNA (piRNA), and small nucleolar RNA (snoRNA). These small RNAs can base pair with a target such as mRNA in a manner that results in the formation of a 3WJ. The base pairing between the effector and target is often incomplete, meaning that the two sequences have some, but not complete, complementarity. Perfect complementarity would result in the formation of a fully double-stranded structure that lacks a 3WJ. In order to form a 3WJ, the effector (e.g., a miRNA) and target (e.g., an mRNA) would generally form a stretch of base pairing of at least 4 nucleotides (nt) followed by a base-pairing stem of at least 4 nt and a loop of unpaired nt, followed by a second stretch of base pairing between the effector and target sequences. The stem-loop can be formed either in the effector or the target RNA. Exemplary 3WJ structures are shown in FIGS. 1, 7, and 9.

The process for the identification of the 3WJ first comprises a computational approach to predict the 3WJ. Sequence databases can be searched for homology using a program such as BLAST. In some embodiments, to identify effector miRNA to target mRNAs, a homology search is performed between a library of miRNA and one particular mRNA of interest (or, in other embodiments, a known group of mRNAs associated with a particular miRNA; or, in other embodiments, all mRNAs are searched) using an annotated database such as the UC Santa Cruz Genome Browser. Public databases of miRNA are available (http://www.mirbase.org; http://mirdb.org/miRDB/; http://www.microrna.org/microma/home.do; http://mirtarbase.mbc.nctu.edu.tw; http://mircancer.ecu.edu; http://www.mir2disease.org; http://zmf.umm.uni-heidelberg.de/apps/zmf/mirwalk2/; http://www.targetscan.org). The effector:target identification search can be done by setting the parameters for the 3WJ including minimal base-pairing of the effector and target, minimal size of the stem and maximum size of the loop as well as the thermostability of the structure. Beyond this trans analysis, identification of cis 3WJs could be done by searching a nucleic acid database (e.g., annotated mRNA) for the ability of individual RNAs to form 3WJs.

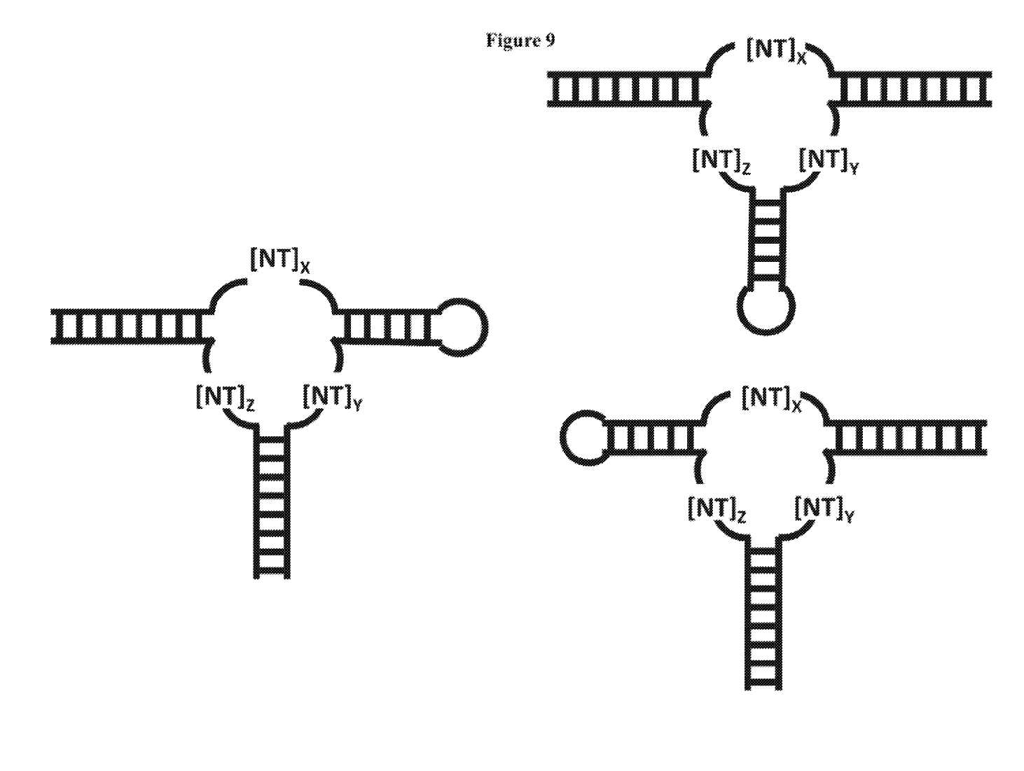

3WJs in nucleic acids form when, in a folded conformation, three double-helical “stems” converge and the stems are connected with one or two loops of varying length. These can be either “perfect” 3WJs in which all nucleotides in the junction take part in the base pairing or can be reduced-symmetry 3WJs where one or more nt at the junction between the three converging steps could be unpaired. The three positions—end of the first effector:target base pairing (x), base of the stem (y) and start of the second effector:target base pairing (y)—can have loops or bulges of unpaired nt (see FIG. 9). In some embodiments, the loop lengths for x, y and z in the 3WJ formed by binding of the small molecule to the target nucleic acid are independently selected from 0, 1, or 2 nt. In some embodiments each loop is independently selected from 0, 1, 2, 3, or 4 nt. In some embodiments each loop is 0 nt. In some embodiments each loop is 1 nt. In some embodiments each loop is 2 nt. In some embodiments one loop is 0 nt. In some embodiments one loop is 1 nt. In some embodiments one loop is 2 nt.

There have been reports of small molecules that bind to DNA and RNA 3WJs, stabilizing that 3D structure (as evidenced by elevation of the melting temperature). Formation of the 3WJ around the small molecule ligand has been reported for triptycenes with single-stranded nucleic acids (Barros & Chenoweth, Angew. Chem. Int. Ed. 2014, 53, 13746-13750; Barros & Chenoweth, Chem. Sci., 2015, 6, 4752-4755; Barros et al., Angew. Chem. Int. Ed. 2016, 55, 1-5), complementary hybridization of two strands around cocaine derivatives (Kent et al., Anal. Chem. 2013, 85, 9916-9923; Sharma & Heemstra, J. Am. Chem. Soc. 2011, 133, 12426-12429; Heemstra, Beilstein J. Org. Chem. 2015, 11, 2713-2720), and the convergence of three strands around symmetric organometallic complexes (Phongtongpasuk et al., Angew. Chem. Int. Ed. 2013, 52, 11513-11516; Oleksi et al., Angew. Chem. Int. Ed. 2006, 45, 1227-1231).

Insofar as essentially all biology can be traced to the classes of targets under consideration, a broad scope of diseases can potentially be addressed by this invention.

In one aspect of the present invention, certain small molecules bring together effector RNAs with regions of target nucleic acid, such as a target RNA, by inducing the formation of and stabilization of 3WJs without the assistance or participation of RNA-binding proteins (RBPs). Many target/effector nucleic acid pairs have multiple potential 3WJ interactions. Thus, small molecule ligands can be “programmed” to modulate the biological functions of essentially any targeted RNA in the cell. This recapitulates the original promise of antisense without the pharmaceutical limitations imposed by administration of exogenous oligonucleotides.

### Targeting Nucleic Acid 3WJs

RNA plays varied and important roles in the cell. Messenger RNAs (mRNAs) are transcribed from protein-encoding genes and are translated to proteins serving a wide range of functions. Since mRNAs adopt structural features such as 3WJs that are critical for their function, some embodiments of the invention provide compounds and methods in which one or more mRNAs are the target nucleic acid. In other embodiments, other types of RNA are a target nucleic acid. For example, ribosomal RNA (rRNA) and transfer RNA (tRNA) function in translation. Noncoding RNAs (ncRNAs) are diverse and abundant, playing regulatory roles in many cellular processes. Long noncoding RNAs (lncRNAs) are RNAs of over 200 nt that do not encode proteins (Morris & Mattick, Nature Reviews Genetics 2014, 15, 423-437; Mattick & Rinn, Nature Struct. & Mol. Biol. 2015, 22, 5-7; Iyer et al., Nature Genetics 2015, 47, 199-208). They can affect the expression of the protein-encoding mRNAs at the level of transcription, splicing, and mRNA decay. lncRNA can regulate transcription by recruiting epigenetic regulators that increase or decrease transcription by altering chromatin structure (Holoch & Moazed, Nat. Rev. Genetics 2015, 16, 71-84). lncRNAs are associated with a wide range of human diseases including cancer, inflammatory diseases, neurological diseases and cardiovascular disease. The targeted modulation of lncRNA could yield, depending on the site of 3WJ formation, up-regulation or down-regulation of the expression of specific genes and proteins for therapeutic benefit. RNA secondary and tertiary structures, in particular 3WJs, are critical for these regulatory activities. Thus, the noncoding transcripts (the noncoding transcriptome) represent a large group of new therapeutic targets. Some exemplary RNA targets and diseases are described in greater detail below.

Targeting mRNA

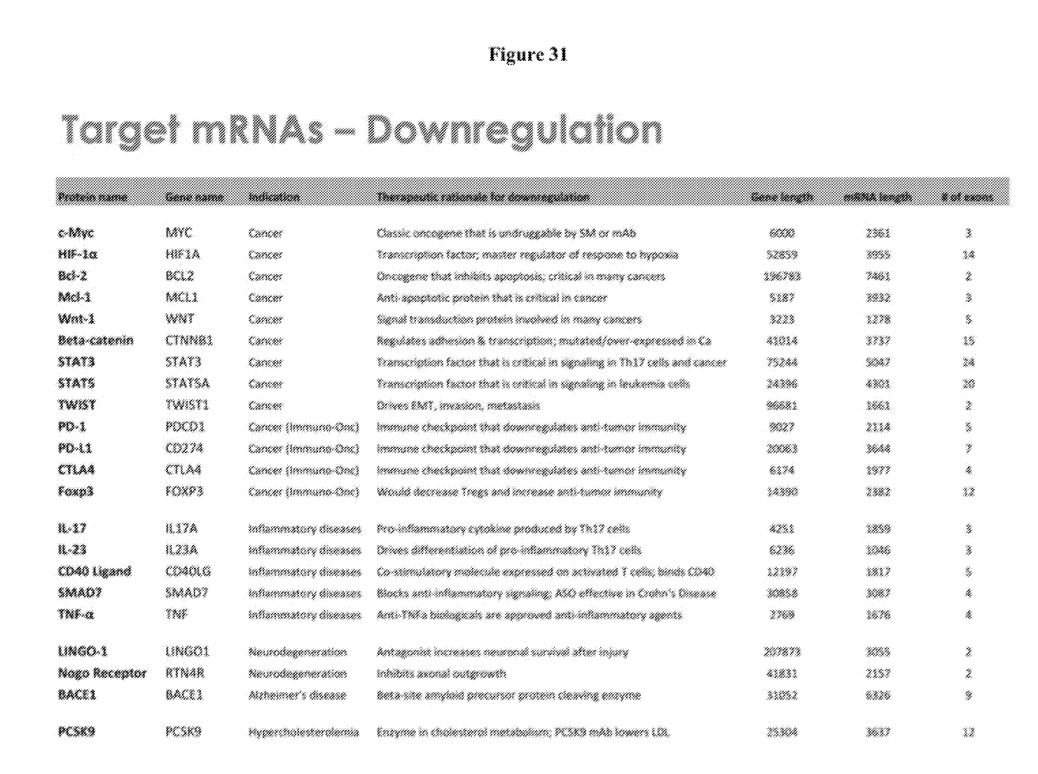

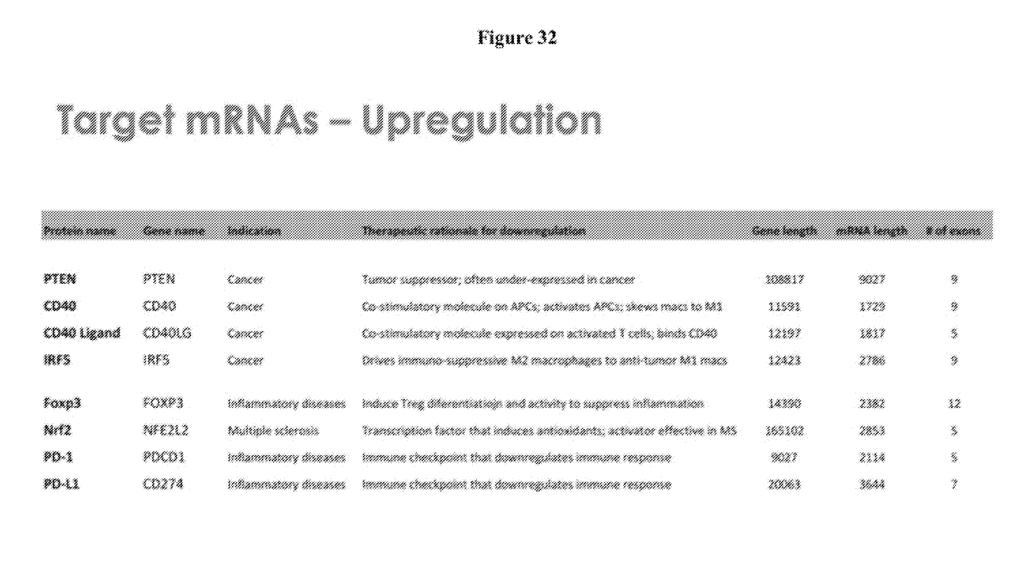

mRNAs are transcribed in the nucleus by RNA polymerase II. There are roughly 20,000 mRNAs in humans. However, there is substantial complexity in that each mRNA can have several different isoforms that may vary in the 5′ start site, the length of the 3′ UTR, and the polyAdenylation (polyA) site usage. Many mRNAs also have a wide variety of alternative splice forms. Typical mRNAs have a cap at the 5′ end, a 5′ UTR, a range of exon and intron numbers, a 3′ UTR and a polyA sequence at the 3′ end. The concentration or “level” of the mRNA, and its resultant protein expression via translation, could be modulated by small molecules that affect the structure of the mRNA, splicing efficiency, stability (half-life) of the mRNA, transport of the RNA, and the efficiency of translation. 3WJ formation could be induced and stabilized in either the coding or noncoding elements to affect mRNA and protein levels. Depending upon the therapeutic goal, the purpose could be either down-regulation or up-regulation. Accordingly, in some embodiments the present invention provides a method of altering the concentration of a target mRNA, comprising the step of contacting the target mRNA with a disclosed compound to form or stabilize a 3WJ comprising a portion of the target mRNA. In some embodiments, forming or stabilizing a 3WJ comprising a portion of the target mRNA affects the splicing efficiency, stability (e.g., half-life), transport, or efficiency of translation of the target mRNA. In some embodiments, the concentration of target mRNA is up-regulated. In some embodiments, the concentration of target mRNA is down-regulated. In some embodiments, the mRNA that is down-regulated is selected from those listed in FIG. 31. In some embodiments, the mRNA that is up-regulated is selected from those listed in FIG. 32. In some embodiments, the 3WJ is cis. In some embodiments, the 3WJ is trans.

Within mRNAs, noncoding regions can affect the level of mRNA and protein expression. Briefly, these include (a) internal ribosome entry sites (IBES) and (b) upstream open reading frames (uORF) in the 5′ UTR that affect translation efficiency, (c) intronic sequences that affect splicing efficiency and alternative splicing patterns, (d) 3′ UTR sequences that affect mRNA and protein localization, and (e) elements in the body of the mRNA and/or the 3′ UTR that control mRNA decay and half-life. (Komar and Hatzoglou, Frontiers Oncol. 5:233, 2015; Weingarten-Gabbay et al., Science 351:pii:aad4939, 2016; Calvo et al., Proc. Natl. Acad. Sci. USA 106:7507-7512; Le Quesne et al., J. Pathol. 220:140-151, 2010; Barbosa et al., PLOS Genetics 9:e10035529, 2013). For example, nearly half of all human mRNAs have uORFs, and many of these reduce the translation of the main ORF. A vast number of single-nucleotide polymorphisms (SNPs) associated with humans are located in these noncoding regions of mRNA, suggesting that they have critical regulatory functions and their targeting by 3WJ formation could impact expression levels. Accordingly, in some embodiments the present invention provides a method of altering the concentration of a target mRNA, comprising the step of contacting the target mRNA with a disclosed compound to form or stabilize a 3WJ comprising a noncoding portion of the target mRNA. In some embodiments, the noncoding portion of the target mRNA is selected from: an internal ribosome entry site (IRES), upstream open reading frames (uORF) in the 5′ UTR, an intronic sequence that affects splicing efficiency and alternative splicing patterns, a 3′ UTR sequence that affects mRNA and protein localization, or an element in the body of the mRNA and/or the 3′ UTR that controls mRNA decay and half-life.

In other embodiments, RNA structures in the 5′ UTR are targeted to inhibit translation. RNA structures such as hairpins in the 5′ UTR have been shown to affect translation. In some embodiments, the invention provides a method of modulating ribosome recognition and/or progression or inhibiting translation in order to decrease the production of a protein, comprising contacting an RNA structure in the 5′ UTR with a disclosed compound. In some embodiments, ribosome recognition and/or progression is modulated or translation is inhibited by formation or stabilization of a 3WJ at or just upstream of the AUG at the start of translation.

The half-life of mRNA is tightly controlled in cells (Palumbo et al., Wiley Interdiscip. Rev. RNA, 2015, 6, 327-336). The concentration of mRNA in cells is a product of tight regulation and the balance between transcription and mRNA decay. Individual mRNAs have their own distinct half-lives, but degradation can be accelerated when mRNA is misprocessed or translation is blocked. The decay of individual mRNAs can be stimulated or inhibited by translational impairment, and, likewise, changes in the half-life of mRNA can alter translational efficiency (Roy & Jacobson, Trends Genet. 2013, 29, 691-699). mRNA stability and degradation can also be controlled by environmental stimuli and biological processes such as cell cycling, cell differentiation, and immune response. mRNA stability is conserved between cell types and species. A majority of human mRNAs exhibit half-lives of less than 8 hours, with a substantial proportion having half-lives of less than 4 hours. Formation and stabilization of a 3WJ in mRNA could significantly increase or decrease mRNA stability. Most mRNA decay takes place by processive 3′ to 5′ and 5′ to 3′ degradation of the RNA by exoribonucleases that degrade mRNA from its ends. These exoribonucleases degrade single-stranded, but not double-stranded, RNA. Accordingly, in some embodiments, the present invention provides a method of increasing the half-life of an mRNA or modulating the level of a protein regulated by the mRNA, comprising contacting the mRNA with a disclosed compound. In some embodiments, the compound induces the formation of or stabilizes a 3WJ in the mRNA, or between the mRNA and a complementary nucleic acid such as another RNA. Without wishing to be bound by theory, the presence of the 3WJ may block the exoribonuclease activity to increase the mRNA half-life and increase the resulting protein level of a protein that would be therapeutically beneficial. In some embodiments, a disclosed compound induces formation of a 3WJ or stabilizes a 3WJ in the 3′ UTR upstream of the polyA site. This may protect a target mRNA from degradation. Alternatively, a 3WJ is introduced in the 5′ UTR provided that it does not inhibit translation.

Termination of mRNA transcripts can occur at different positions, thus forming mRNAs with distinct properties including alterative protein coding. mRNA processing occurs co-transcriptionally, such that slowdown of RNA polymerase II processivity is linked to 3′ end cleavage and polyadenylation. Protein-coding mRNAs of varying lengths can be created by alternative termination and polyA site usage. These distinct RNAs can have different half-lives, translational efficiencies or subcellular localization. In some embodiments, a disclosed compound binds to and/or stabilizes a 3WJ at a site of RNA polymerase II termination and/or polyA addition. In some embodiments, such 3WJ formation and/or stabilization alters the function of the mRNA.

One consideration for the selection of targets and effectors is their relative expression levels. In some embodiments, the effector RNA concentration is similar to the target nucleic acid (e.g., a target mRNA) concentration. If the abundance of the target greatly exceeds the effector, then a functional impact on the RNA target is rendered more challenging. In certain embodiments, the relative concentration of the effector:target nucleic acid in the biological system (e.g. subject's whole body, tissue, cell, nucleus, mitochondria, or cytoplasm) is between 100:1 to 1:100. In some embodiments, the relative concentration is 50:1 to 1:50, 25:1 to 1:25, 10:1 to 1:10, 5:1 to 1:5, 3:1 to 1:3, 2:1 to 1:2 or approximately 1:1. In some embodiments, the effector:target concentration is at least about 10:1. In some embodiments, the effector:target concentration is at least about 25:1. In addition, in certain embodiments both RNAs are localized to the same compartment of the cell such that an interaction is more likely. While the subcellular distribution need not match precisely, it should not be exclusive. For example, the effector should not be exclusively nuclear while the target nucleic acid is exclusively cytoplasmic.

Small molecules can be used to modulate splicing of pre-mRNA for therapeutic benefit in a variety of settings. One example is spinal muscular atrophy (SMA). SMA is a consequence of insufficient amounts of the survival of motor neuron (SMN) protein. Humans have two versions of the SMN gene, SMN1 and SMN2. SMA patients have a mutated SMN1 gene and thus rely solely on SMN2 for their SMN protein. The SMN2 gene has a silent mutation in exon 7 that causes inefficient splicing such that exon 7 is skipped in the majority of SMN2 transcripts, leading to the generation of a defective protein that is rapidly degraded in cells, thus limiting the amount of SMN protein produced from this locus. A small molecule that promotes the efficient inclusion of exon 7 during the splicing of SMN2 transcripts would be an effective treatment for SMA (Palacino et al., Nature Chem. Biol., 2015, 11, 511-517). Accordingly, in one aspect, the present invention provides a method of identifying a small molecule that modulates the splicing, transcription, or cellular half-life of a target pre-mRNA to treat a disease or disorder, comprising the steps of: screening one or more small molecules disclosed herein for binding to a 3WJ in the target pre-mRNA; and identifying which small molecule(s) bind to the 3WJ and modulate the splicing, transcription, or cellular half-life of the target pre-mRNA to treat the disease or disorder. In some embodiments, the method further comprises identifying the target pre-mRNA according to a disclosed computational survey. In some embodiments, the target pre-mRNA is capable of forming a 3WJ in the presence of an effector RNA such as an effector miRNA. In some embodiments, the small molecule binds to a trans 3WJ formed between the effector RNA and the target pre-mRNA. In some embodiments, the small molecule binds to a cis 3WJ formed between portions of the target pre-mRNA. In some embodiments, the pre-mRNA is an SMN2 transcript. In some embodiments, the disease or disorder is spinal muscular atrophy (SMA).

Even in cases in which defective splicing does not cause the disease, alteration of splicing patterns can be used to correct the disease. Nonsense mutations leading to premature translational termination can be eliminated by exon skipping if the exon sequences are in-frame. This can create a protein that is at least partially functional. One example of the use of exon skipping is the dystrophin gene in Duchenne muscular dystrophy (DMD). A variety of different mutations leading to premature termination codons in DMD patients can be eliminated by exon skipping promoted by oligonucleotides (reviewed in Fairclough et al., Nature Rev. Gen., 2013, 14, 373-378). Small molecules that bind RNA structures and affect splicing are expected to have a similar effect. Accordingly, in one aspect, the present invention provides a method of identifying a small molecule that modulates the splicing pattern of a target pre-mRNA to treat a disease or disorder, comprising the steps of: Accordingly, in one aspect, the present invention provides a method of identifying a small molecule that modulates the splicing pattern of a target pre-mRNA to treat a disease or disorder, comprising the steps of: screening one or more small molecules disclosed herein for binding to a 3WJ in the target pre-mRNA; and identifying which small molecule(s) bind to the 3WJ and modulate the splicing pattern of the target pre-mRNA to treat the disease or disorder. In some embodiments, the method further comprises identifying the target pre-mRNA according to a disclosed computational survey. In some embodiments, the target pre-mRNA is capable of forming a 3WJ in the presence of an effector RNA such as an effector miRNA. In some embodiments, the small molecule binds to a trans 3WJ formed between the effector RNA and the target pre-mRNA. In some embodiments, the small molecule binds to a cis 3WJ formed between portions of the target pre-mRNA. In some embodiments, the target pre-mRNA is a dystrophin gene transcript. In some embodiments, the small molecule promotes exon skipping to eliminate premature translational termination. In some embodiments, the disease or disorder is Duchenne muscular dystrophy (DMD).

The present invention contemplates the use of small molecules to up- or down-regulate the expression of specific proteins based on targeting 3WJs in their cognate mRNAs. Accordingly, the present invention provides methods of modulating the downstream protein expression associated with a target mRNA comprising the step of contacting the target mRNA with a small molecule disclosed herein that binds to or stabilizes a 3WJ in the target mRNA. In another aspect, the present invention provides a method of producing a small molecule that modulates the downstream protein expression associated with a target mRNA, comprising the steps of: screening one or more small molecules disclosed herein for binding to a 3WJ in the target mRNA; and identifying which small molecule(s) bind to the 3WJ and modulate the downstream protein expression associated with the target mRNA. In some embodiments, the method further comprises identifying the target mRNA according to a disclosed computational survey. In some embodiments, the target mRNA is capable of forming a 3WJ in the presence of an effector RNA such as an effector miRNA. In some embodiments, the small molecule binds to a trans 3WJ formed between the effector RNA and the target mRNA. In some embodiments, the small molecule binds to a cis 3WJ formed between portions of the target mRNA. In some embodiments, modulation of the downstream protein expression associated with the target mRNA treats or ameliorates a disclosed disease or condition.

In some embodiments, the present invention provides a method of treating a disease or disorder mediated by mRNA, comprising the step of administering to a patient in need thereof a small molecule disclosed herein.

### Targeting Regulatory RNA

The largest set of RNA targets comprises RNA that is transcribed but not translated into protein, termed “non-coding RNA”. Non-coding RNA is highly conserved and the many varieties of non-coding RNA play a wide range of regulatory functions. The term “non-coding RNA,” as used herein, includes but is not limited to micro-RNA (miRNA), long non-coding RNA (lncRNA), long intergenic non-coding RNA (lincRNA), Piwi-interacting RNA (piRNA), competing endogenous RNA (ceRNA), and pseudo-genes. Each of these sub-categories of non-coding RNA offers a large number of RNA targets with significant therapeutic potential. Accordingly, in one aspect, the present invention provides a method of producing a small molecule that modulates a regulatory function of a target non-coding RNA, comprising the steps of: screening one or more small molecules disclosed herein for binding to a 3WJ in the target non-coding RNA; and identifying which small molecule(s) bind to the 3WJ and modulate the regulatory function of the target non-coding RNA. In some embodiments, the method further comprises identifying the target non-coding RNA according to a disclosed computational survey. In some embodiments, the target non-coding RNA is capable of forming a 3WJ in the presence of an effector RNA such as an effector miRNA. In some embodiments, the small molecule binds to a trans 3WJ formed between the effector RNA and the target non-coding RNA. In some embodiments, the small molecule binds to a cis 3WJ formed between portions of the target non-coding RNA. In some embodiments, modulating the regulatory function of the target non-coding RNA treats or ameliorates a disease caused by a miRNA, lncRNA, lincRNA, piRNA, ceRNA, or pseudo-gene.

Targeting miRNA

miRNAs are short double-strand RNAs that regulate gene expression (see Elliott & Ladomery, Molecular Biology of RNA, 2^{nd} Ed.). Each miRNA can affect the expression of many human genes. There are nearly 2,000 miRNAs in humans. These RNAs regulate many biological processes, including cell differentiation, cell fate, motility, survival, and function. miRNA expression levels vary between different tissues, cell types, and disease settings. They are frequently aberrantly expressed in tumors versus normal tissue, and their activity may play significant roles in cancer (for reviews, see Croce, Nature Rev. Genet. 10:704-714, 2009; Dykxhoorn Cancer Res. 70:6401-6406, 2010). miRNAs have been shown to regulate oncogenes and tumor suppressors and themselves can act as oncogenes or tumor suppressors. Some have been shown to promote epithelial-mesenchymal transition (EMT) and cancer cell invasiveness and metastasis. In the case of oncogenic miRNAs, their inhibition could be an effective anti-cancer treatment. Accordingly, in one aspect, the present invention provides a method of producing a small molecule that modulates the activity of a target miRNA to treat a disease or disorder, comprising the steps of: screening one or more small molecules disclosed herein for binding to a 3WJ comprising at least a portion of the target miRNA; and identifying which small molecule(s) bind to the 3WJ and modulate activity of the target miRNA to treat the disease or disorder. In some embodiments, the method further comprises identifying the target miRNA according to a disclosed computational survey. In some embodiments, the target miRNA is capable of forming a 3WJ in the presence of an RNA to which it binds, such as an mRNA. In some embodiments, the small molecule binds to a trans 3WJ formed between the target miRNA and an RNA to which it binds. In some embodiments, the miRNA regulates an oncogene or tumor suppressor, or acts as an oncogene or tumor suppressor. In some embodiments, the disease is cancer. In some embodiments, the cancer is a solid tumor.

In some embodiments, the target miRNA is an oncogenic miRNA such as miR-155, miR-17˜92, miR-19, miR-21, or miR-10b (see Stahlhut & Slack, Genome Med. 2013, 5, 111). miR-155 plays pathological roles in inflammation, hypertension, heart failure, and cancer. In cancer, miR-155 triggers oncogenic cascades and apoptosis resistance, as well as increasing cancer cell invasiveness. Altered expression of miR-155 has been described in multiple cancers, reflecting staging, progress and treatment outcomes. Cancers in which miR-155 over-expression has been reported include breast cancer, thyroid carcinoma, colon cancer, cervical cancer, and lung cancer. It is reported to play a role in drug resistance in breast cancer. miR-17˜92 (also called Oncomir-1) is a polycistronic 1 kb primary transcript comprising miR-17, 20a, 18a, 19a, 92-1 and 19b-1. It is activated by MYC. miR-19 alters the gene expression and signal transduction pathways in multiple hematopoietic cells, and it triggers leukemogenesis and lymphomagenesis. It is implicated in a wide variety of human solid tumors and hematological cancers. miR-21 is an oncogenic miRNA that reduces the expression of multiple tumor suppressors. It stimulates cancer cell invasion and is associated with a wide variety of human cancers including breast, ovarian, cervix, colon, lung, liver, brain, esophagus, prostate, pancreas, and thyroid cancers. Accordingly, in some embodiments of the methods described above, the target miRNA is selected from miR-155, miR-17˜92, miR-19, miR-21, or miR-10b. In some embodiments, the disease or disorder is a cancer selected from breast cancer, ovarian cancer, cervical cancer, thyroid carcinoma, colon cancer, liver cancer, brain cancer, esophageal cancer, prostate cancer, lung cancer, leukemia, or lymph node cancer. In some embodiments, the cancer is a solid tumor.

Beyond oncology, miRNAs play roles in many other diseases including cardiovascular and metabolic diseases (Quiant and Olson, J. Clin. Invest. 123:11-18, 2013; Olson, Science Trans. Med. 6: 239ps3, 2014; Baffy, J. Clin. Med. 4:1977-1988, 2015).

Many mature miRNAs are relatively short in length and thus may lack sufficient folded, three-dimensional structure to be targeted by small molecules. However, it is believed that the levels of such miRNA could be reduced by small molecules that bind a 3WJ comprising at least a portion of the primary transcript or the pre-miRNA to block the biogenesis of the mature miRNA. Accordingly, in some embodiments of the methods described above, the target miRNA is a primary transcript or pre-miRNA.

lncRNA are RNAs of over 200 nucleotides (nt) that do not encode proteins (see Rinn & Chang, Ann. Rev. Biochem. 2012, 81, 145-166; (for reviews, see Morris and Mattick, Nature Reviews Genetics 15:423-437, 2014; Mattick and Rinn, Nature Structural & Mol. Biol. 22:5-7, 2015; Iyer et al., Nature Genetics 47(:199-208, 2015)). They can affect the expression of the protein-encoding mRNAs at the level of transcription, splicing and mRNA decay. Considerable research has shown that lncRNA can regulate transcription by recruiting epigenetic regulators that increase or decrease transcription by altering chromatin structure (e.g., Holoch and Moazed, Nature Reviews Genetics 16:71-84, 2015). lncRNAs are associated with human diseases including cancer, inflammatory diseases, neurological diseases and cardiovascular disease (for instance, Presner and Chinnaiyan, Cancer Discovery 1:391-407, 2011; Johnson, Neurobiology of Disease 46:245-254, 2012; Gutscher and Diederichs, RNA Biology 9:703-719, 2012; Kumar et al., PLOS Genetics 9:e1003201, 2013; van de Vondervoort et al., Frontiers in Molecular Neuroscience, 2013; Li et al., Int. J. Mol. Sci. 14:18790-18808, 2013). The targeting of lncRNA could be done to up-regulate or down-regulate the expression of specific genes and proteins for therapeutic benefit (e.g., Wahlestedt, Nature Reviews Drug Discovery 12:433-446, 2013; Guil and Esteller, Nature Structural & Mol. Biol. 19:1068-1075, 2012). In general, lncRNAs are expressed at lower levels relative to mRNAs. Many lncRNAs are physically associated with chromatin (Werner et al., Cell Reports 12, 1-10, 2015) and are transcribed in close proximity to protein-encoding genes. They often remain physically associated at their site of transcription and act locally, in cis, to regulate the expression of a neighboring mRNA. lncRNAs regulate the expression of protein-encoding genes, acting at multiple different levels to affect transcription, alternative splicing and mRNA decay. For example, lncRNA has been shown to bind to the epigenetic regulator PRC2 to promote its recruitment to genes whose transcription is then repressed via chromatin modification. lncRNA may form complex structures such as 3WJ that mediate their association with various regulatory proteins. A small molecule that binds to these lncRNA structures could be used to modulate the expression of genes that are normally regulated by an individual lncRNA. The mutation and dysregulation of lncRNA is associated with human diseases; therefore, there are a multitude of lncRNAs that could be therapeutic targets. Accordingly, in some embodiments of the methods described above, the target non-coding RNA is a lncRNA. In some embodiments, the lncRNA is associated with a cancer, inflammatory disease, neurological disease, or cardiovascular disease. In some embodiments, the method further comprises identifying the target lncRNA according to a disclosed computational survey. In some embodiments, the target lncRNA is capable of forming a 3WJ in the presence of an effector RNA such as an effector miRNA. In some embodiments, the small molecule binds to a trans 3WJ formed between the effector RNA and the target lncRNA. In some embodiments, the small molecule binds to a cis 3WJ formed between portions of the target lncRNA.

One examplary target lncRNA is HOTAIR, a lncRNA expressed from the HoxC locus on human chromosome 12. Its expression level is low (˜100 RNA copies per cell). Unlike many lncRNAs, HOTAIR can act in trans to affect the expression of distant genes. It binds the epigenetic repressor PRC2 as well as the LSD1/CoREST/REST complex, another repressive epigenetic regulator (Tsai et al., Science 329, 689-693, 2010). HOTAIR is a highly structured RNA with over 50% of its nucleotides being involved in base pairing. It is frequently dysregulated (often up-regulated) in various types of cancer (Yao et al., Int. Mol. Sci. 15:18985-18999, 2014; Deng et al., PLOS One 9:e110059, 2014). Cancer patients with high expression levels of HOTAIR have a significantly poorer prognosis, compared with those with low expression levels. HOTAIR has been reported to be involved in the control of apoptosis, proliferation, metastasis, angiogenesis, DNA repair, chemoresistance and tumor cell metabolism. It is highly expressed in metastatic breast cancers. High levels of expression in primary breast tumors are a significant predictor of subsequent metastasis and death. HOTAIR also has been reported to be associated with esophageal squamous cell carcinoma, and it is a prognostic factor in colorectal cancer, cervical cancer, gastric cancer and endometrial carcinoma. Therefore, HOTAIR-binding small molecules are novel anti-cancer drug candidates. Accordingly, in some embodiments of the methods described above, the target non-coding RNA is HOTAIR. In some embodiments, the small molecule binds to a 3WJ in the HOTAIR structure. In some embodiments, the disease or disorder is breast cancer, esophageal squamous cell carcinoma, colorectal cancer, cervical cancer, gastric cancer, or endometrial carcinoma.

Another potential cancer target among lncRNA is MALAT-1 (metastasis-associated lung adenocarcinoma transcript 1), also known as NEAT2 (nuclear-enriched abundant transcript 2) (Gutschner et al, Cancer Res. 73:1180-1189, 2013; Brown et al, Nat. Structural & Mol. Biol. 21:633-640, 2014). It is a highly conserved 7 kb nuclear lncRNA that is localized in nuclear speckles. It is ubiquitously expressed in normal tissues, but is up-regulated in many cancers. MALAT-1 is a predictive marker for metastasis development in multiple cancers including lung cancer. It appears to function as a regulator of gene expression, potentially affecting transcription and/or splicing. MALAT-1 knockout mice have no phenotype, indicating that it has limited normal function. However, MALAT-1-deficient cancer cells are impaired in migration and form fewer tumors in a mouse xenograft tumor models. Antisense oligonucleotides (ASO) blocking MALAT-1 prevent metastasis formation after tumor implantation in mice. Some mouse xenograft tumor model data indicates that MALAT-1 knockdown by ASOs may inhibit both primary tumor growth and metastasis. Thus, a small molecule targeting MALAT-1 is expected to be effective in inhibiting tumor growth and metastasis. Accordingly, in some embodiments of the methods described above, the target non-coding RNA is MALAT-1. In some embodiments, the small molecule binds to a 3WJ in the MALAT-1 structure. In some embodiments, the disease or disorder is a cancer in which MALAT-1 is up-regulated, such as lung cancer.

In some embodiments, the present invention provides a method of treating a disease or disorder mediated by non-coding RNA (such as HOTAIR or MALAT-1), comprising the step of administering to a patient in need thereof a compound of the present invention. Such compounds are described in detail herein.

### Targeting Toxic RNA (Repeat RNA)

Simple repeats in mRNA often are associated with human disease. These are often, but not exclusively, repeats of three nucleotides such as CAG (“triplet repeats”) (for reviews, see Gatchel and Zoghbi, Nature Reviews Genetics 6:743-755, 2005; Krzyzosiak et al., Nucleic Acids Res. 40:11-26, 2012; Budworth and McMurray, Methods Mol. Biol. 1010:3-17, 2013). Triplet repeats are abundant in the human genome, and they tend to undergo expansion over generations. Approximately 40 human diseases are associated with the expansion of repeat sequences. Diseases caused by triplet expansions are known as Triplet Repeat Expansion Diseases (TRED). Healthy individuals have a variable number of triplet repeats, but there is a threshold beyond which a higher repeat number causes disease. The threshold varies in different disorders. The triplet repeat can be unstable. As the gene is inherited, the number of repeats may increase, and the condition may be more severe or have an earlier onset from generation to generation. When an individual has a number of repeats in the normal range, it is not expected to expand when passed to the next generation. When the repeat number is in the premutation range (a normal, but unstable repeat number), then the repeats may or may not expand upon transmission to the next generation. Normal individuals who carry a premutation do not have the condition, but are at risk of having a child who has inherited a triplet repeat in the full mutation range and who will be affected. TREDs can be autosomal dominant, autosomal recessive or X-linked. The more common triplet repeat disorders are autosomal dominant.

The repeats can be in the coding or noncoding portions of the mRNA. In the case of repeats within noncoding regions, the repeats may lie in the 5′ UTR, introns, or 3′ UTR sequences. Some examples of diseases caused by repeat sequences within coding regions are shown in Table 1.

TABLE 1
Repeat Expansion Diseases in Which the Repeat
Resides in the Coding Regions of mRNA

| | | | Normal repeat | Disease repeat |
|---|---|---|---|---|
| Disease | Gene | Repeat | number | number |
| HD | HTT | CAG | 6-35 | 36-250 |
| DRPLA | ATN1 | CAG | 6-35 | 49-88 |
| SBMA | AR | CAG | 9-36 | 38-62 |
| SCA1 | ATXN1 | CAG | 6-35 | 49-88 |
| SCA2 | ATXN2 | CAG | 14-32 | 33-77 |
| SCA3 | ATXN3 | CAG | 12-40 | 55-86 |
| SCA6 | CACNA1A | CAG | 4-18 | 21-30 |
| SCA7 | ATXN7 | CAG | 7-17 | 38-120 |
| SCA17 | TBP | CAG | 25-42 | 47-63 |

Some examples of diseases caused by repeat sequences within noncoding regions of mRNA are shown in Table 2.

TABLE 2
Repeat Expansion Diseases in Which the Repeat
Resides in the Noncoding Regions of mRNA

| | | | | Normal | Disease |
|---|---|---|---|---|---|
| | | | Repeat | repeat | repeat |
| Disease | Gene | Repeat | location | number | number |
| Fragile X | FMR1 | CGG | 5′ UTR | 6-53 | ≥230 |
| DM1 | DMPK | CTG | 3′ UTR | 5-37 | ≥50 |
| FRDA | FXN | GAA | Intron | 7-34 | ≥100 |
| SCA8 | ATXN8 | CTG | Noncoding | 16-37 | 110-250 |
| | | | antisense | | |
| SCA10 | ATXN10 | ATTCT | Intron | 9-32 | 800-4500 |
| SCA12 | PPP2R2B | CAG | 5′ UTR | 7-28 | 66-78 |
| C9FTD/ALS | C9orf72 | GGGGCC | Intron | ~30 | 100s |

The toxicity that results from the repeat sequence can be direct consequence of the action of the toxic RNA itself, or, in cases in which the repeat expansion is in the coding sequence, due to the toxicity of the RNA and/or the aberrant protein. The repeat expansion RNA can act by sequestering critical RNA-binding proteins (RBP) into foci. One example of a sequestered RBP is the Muscleblind family protein MBNL1. Sequestration of RBPs leads to defects in splicing as well as defects in nuclear-cytoplasmic transport of RNA and proteins. Sequestration of RBPs also can affect miRNA biogenesis. These perturbations in RNA biology can profoundly affect neuronal function and survival, leading to a variety of neurological diseases.

Repeat sequences in RNA form secondary and tertiary structures that bind RBPs and affect normal RNA biology. One specific example disease is myotonic dystrophy (DM1; dystrophia myotonica), a common inherited form of muscle disease characterized by muscle weakness and slow relaxation of the muscles after contraction (Machuca-Tzili et al., Muscle Nerve 32:1-18, 2005). It is caused by a CUG expansion in the 3′ UTR of the dystrophia myotonica protein kinase (DMPK) gene. This repeat-containing RNA causes the misregulation of alternative splicing of several developmentally regulated transcripts through effects on the splicing regulators MBNL1 and the CUG repeat binding protein (CELF1) (Wheeler et al., Science 325:336-339, 2009). Small molecules that bind the CUG repeat within the DMPK transcript would alter the RNA structure and prevent focus formation and alleviate the effects on these spicing regulators. Fragile X Syndrome (FXS), the most common inherited form of mental retardation, is the consequence of a CGG repeat expansion within the 5′ UTR of the FMR1 gene (Lozano et al., Intractable Rare Dis. Res. 3:134-146, 2014). FMRP is critical for the regulation of translation of many mRNAs and for protein trafficking, and it is an essential protein for synaptic development and neural plasticity. Thus, its deficiency leads to neuropathology. A small molecule targeting this CGG repeat RNA may alleviate the suppression of FMR1 mRNA and FMRP protein expression. Another TRED having a very high unmet medical need is Huntington's disease (HD). HD is a progressive neurological disorder with motor, cognitive, and psychiatric changes (Zuccato et al., Physiol Rev. 90:905-981, 2010). It is characterized as a poly-glutamine or polyQ disorder since the CAG repeat within the coding sequence of the HTT gene leads to a protein having a poly-glutamine repeat that appears to have detrimental effects on transcription, vesicle trafficking, mitochondrial function, and proteasome activity. However, the HTT CAG repeat RNA itself also demonstrates toxicity, including the sequestration of MBNL1 protein into nuclear inclusions. One other specific example is the GGGGCC repeat expansion in the C9orf72 (chromosome 9 open reading frame 72) gene that is prevalent in both familial frontotemporal dementia (FTD) and familial amyotrophic lateral sclerosis (ALS) (Ling et al., Neuron 79:416-438, 2013; Haeusler et al., Nature 507:195-200, 2014). The repeat RNA structures form nuclear foci that sequester critical RNA binding proteins. The GGGGCC repeat RNA also binds and sequesters RanGAP1 to impair nucleocytoplasmic transport of RNA and proteins (Zhang et al., Nature 525:56-61, 2015). Selectively targeting any of these repeat expansion RNAs could add therapeutic benefit in these neurological diseases.

There is some evidence that triplet repeats adopt 3WJ structures, and as such are suitable targets for disclosed small molecules. Without wishing to be bound by theory, it is believed that the repeat nucleotide sequences in certain repeat diseases organize themselves into transient 3WJ such as slipped 3WJ formed by (CAG)-(CTG) repeats. Such 3WJ could be stabilized by small molecules of the present invention and their toxic effects decreased in order to treat a TRED such as those described above or in Tables 1 and 2. (See, e.g., Barros et al., Chem. Sci. 2015, 6, 4752-4755.)

The present invention contemplates a method of treating a disease or disorder wherein aberrant RNAs themselves cause pathogenic effects, rather than acting through the agency of protein expression or regulation of protein expression. In some embodiments, the disease or disorder is mediated by repeat RNA, such as those described above or in Tables 1 and 2. In some embodiments, the disease or disorder is a repeat expansion disease in which the repeat resides in the coding regions of mRNA. In some embodiments, the disease or disorder is a repeat expansion disease in which the repeat resides in the noncoding regions of mRNA. In some embodiments, the disease or disorder is selected from Huntington's disease (HD), dentatorubral-pallidoluysian atrophy (DRPLA), spinal-bulbar muscular atrophy (SBMA), or a spinocerebellar ataxia (SCA) selected from SCA1, SCA2, SCA3, SCA6, SCAT, or SCA17. In some embodiments, the disease or disorder is selected from Fragile X Syndrome, myotonic dystrophy (DM1 or dystrophia myotonica), Friedreich's Ataxia (FRDA), a spinocerebellar ataxia (SCA) selected from SCAB, SCA10, or SCA12, or C9FTD (amyotrophic lateral sclerosis or ALS).

In some embodiments, the disease is amyotrophic lateral sclerosis (ALS), Huntington's disease (HD), frontotemporal dementia (FTD), myotonic dystrophy (DM1 or dystrophia myotonica), or Fragile X Syndrome.

In some embodiments, the present invention provides a method of treating a disease or disorder mediated by repeat RNA, comprising the step of administering to a patient in need thereof a compound of the present invention. Such compounds are described in detail herein.

Also provided is a method of producing a small molecule that modulates the activity of a target repeat expansion RNA, comprising the steps of: screening one or more small molecules disclosed herein for binding to a 3WJ in the target repeat expansion RNA; and identifying which small molecule(s) bind to the 3WJ and modulate the activity of the target repeat expansion RNA. In some embodiments, the method further comprises identifying the target repeat expansion RNA according to a disclosed computational survey. In some embodiments, the target repeat expansion RNA is capable of forming a 3WJ in the presence of an effector RNA such as an effector miRNA. In some embodiments, the small molecule binds to a trans 3WJ formed between the effector RNA and the target repeat expansion RNA. In some embodiments, the small molecule binds to a cis 3WJ formed between portions of the target repeat expansion RNA. In some embodiments, the repeat expansion RNA causes a disease or disorder selected from HD, DRPLA, SBMA, SCA1, SCA2, SCA3, SCA6, SCAT, or SCA17. In some embodiments, the disease or disorder is selected from Fragile X Syndrome, DM1, FRDA, SCAB, SCA10, SCA12, or C9FTD. In some embodiments, the small molecule is effective to treat or ameliorate the disease or disorder.

### Other Target RNAs and Diseases/Conditions

An association exists between a large number of additional RNAs and diseases or conditions, some of which are shown below in Table 3. Accordingly, in some embodiments of the methods described above, the target RNA is selected from those in Table 3. In some embodiments, the disease or disorder is selected from those in Table 3.

TABLE 3
Target RNAs and Associated Diseases/Conditions

| RNA Target | Indication |
|---|---|
| A20 | inflammatory diseases; liver failure; liver |
| | transplant |
| ABCA1 | coronary artery disease |
| ABCB11 | Primary Biliary Sclerosis |
| ABCB4 | Primary Biliary Sclerosis |
| ABCG5/8 | coronary artery disease |
| Adiponectin | diabetes; obesity; metabolic syndrome |
| AMPK | diabetes |
| ApoA1 | hypercholesterolemia |
| ApoA5 | hypercholesterolemia |
| ApoC3 | chylomicronemia syndrome |
| AR | prostate cancer |
| ARlnc-1 | prostate cancer |
| ATXN1 | spinocerebellar ataxia 1 |
| ATXN10 | spinocerebellar ataxia 10 |
| ATXN2 | spinocerebellar ataxia 2 |
| ATXN3 | spinocerebellar ataxia 3 |
| ATXN7 | spinocerebellar ataxia 7 |
| ATXN8 | spinocerebellar ataxia 8 |
| BACE1 | AD |
| BCL2 | cancer |
| BCR/ABL | CML |
| BDNF | Huntington's Disease |
| Beta-catenin | cancer |
| BRAF | cancer |
| BRCA1 | cancer |
| BRD4 | cancer |
| BTK | cancer |
| C9orf72 (ALS, FTD) | ALS, FTD |
| CACNA1A | spinocerebellar ataxia 6 |
| CD274 | tumor immunology |
| CD279 | tumor immunology |
| CD3zeta | inflammation and autoimmune diseases |
| CD40LG | inflammation |
| CFTR | Cystic Fibrosis |
| cKIT | GIST; mastocytoma |
| CNTF | macular degeneration |
| Complement Factor H | macular degeneration |
| CRACM1 | inflammatory diseases; autoimmune disease; |
| | organ transplant |
| CTLA4 | cancer; inflammatory diseases |
| DGAT2 | NASH |
| DIO2 | dyslipidemia |
| Dystrophin | Duchenne Muscular Dystrophy; Becker's |
| | Muscular Dystrophy |
| EGFR | cancer |
| EIF4E | cancer |
| EZH2 | cancer |
| Factor 7 | hemophilia |
| Factor 8 | hemophilia |
| Factor 9 | hemophilia |
| Fetal Hemoglobin | sickle cell anemia; beta-thalassemia |
| FLT3 | AML |
| FMR1 | Fragile X Syndrome |
| Foxp3 | inflammation & autoimmune diseases |
| Frataxin | Friedreich's Ataxia |
| HAMP/Hepcidin | thalassemia; hereditary hemochromatosis |
| HER2 | cancer |
| HIF-1a | cancer |
| HOTAIR | cancer |
| HTT | Huntington's Disease |
| IL-1 | rheumatoid arthritis |
| IL-17 | inflammatory & autoimmune diseases |
| IL-23 | inflammatory & autoimmune diseases |
| IL-6 | rheumatoid arthritis |
| Ipf1/Pdx1 | diabetes |
| KRAS | cancer |
| Laminin-1a | Merosin-deficient congenital muscular dystrophy |
| | MDCA1 |
| LARGE | Muscular Dystroglycanopathy Type B, 6 |
| LDLR | hypercholesterolemia |
| LINGO1 | neurodegeneration |
| MALAT1 | cancer |
| MAX | cancer |
| MBNL1 | Myotonic Dystrophy |
| MCL1 | cancer |
| MECP2 | Rett Syndrome |
| Mertk | Lupus |
| miR-103 | NASH |
| miR-107 | NASH |
| miR-10b | GBM |
| miR-155 | ALS and others |
| miR-21 | solid tumors |
| miR-221 | HCC |
| mTOR | cancer |
| MYC | cancer |
| Nanog | neurological diseases |
| NF1 | neurofibromatosis |
| Nrf2 | multiple sclerosis |
| PAH | phenylketonuria |
| PCSK6 | hypertension |
| PCSK9 | hypercholesterolemia |
| PD-1 | cancer; inflammation |
| PD-L1 | cancer; inflammation |
| PDK1/2 | Polycystic kidney disease |
| PGC1-a/FNDC5 | PGC1-a/FNDC5 |
| Progranulin | neurological diseases |
| PTB-1B | diabetes |
| PTEN | cancer |
| PTPN1 | Type II diabetes |
| r(AUUCU)^{exp} | SCA10 |
| r(CAG)^{exp} | Huntington's Disease |
| r(CCUG)^{exp} | DM2 |
| r(CGG)^{exp} | FXTAS |
| r(CUG)^{exp} | DM1 |
| r(GGGGCC)^{exp} | c9ALS (familial) |
| r(GGGGCC)^{cxp} | c9FTD |
| Ras | Cancer |
| RORC | autoimmune disease |
| RTN4 | Neurodegeneration |
| RTN4R | Neurodegeneration |
| Sarcospan | Duchenne Muscular Dystrophy |
| Serca2a | congestive heart failure |
| SirT6 | Cancer |
| SMAD7 | IBD |
| SMN2 | Spinal Muscular Atrophy |
| SNCA | AD |
| SORT1 | coronary artery disease |
| SRBI | coronary artery disease |
| STAT3 | Cancer |
| STAT5 | Cancer |
| T-bet | Cancer |
| Thyroid Hormone | dyslipidemia; NASH; NAFLD |
| Receptor beta | |
| TIM-3 | inflammatory diseases; cancer |
| TNFa | inflammatory disease |
| TNFRSF11A | Osteoporosis |
| TNFSF11 | Osteoporosis |
| TRIB1 | coronary artery disease |
| TTR | Amyloidosis |
| TWIST1 | Cancer |
| Utrophin | Duchenne Muscular Dystrophy |
| Wnt | Cancer |

### Targeting Viral RNAs

In an aspect of the invention, the compounds and disclosed methods are used to target a viral nucleic acid or a transcript thereof. In some embodiments, the virus has an RNA genome. Both single-strand RNA and double-strand RNA viruses have double-stranded RNA sequences that may be selected as targets for modulation. For viruses such as HCV (Pirakitikulr et al., Mol. Cell 2016, 61, 1-10) and HIV-1 (Lavender et al., PLOS Comp. Bio. 2015, 11), a substantial amount of RNA structural information exists, and 3WJs are present in the structures of the viral RNA genomes. Many viruses have RNA structures or genetic elements that are rarely found or not found in mammalian genomes. Thus, targeting these elements provides selective antiviral agents with minimal effect on host processes. These genetic elements include RNA sequences that mediate translation, such as IRES elements that are far more common and functionally significant in viruses than in the mammalian genome. Unique RNA sequences also can be essential for the packaging of the RNA into a virus particle. Accordingly, in some embodiments, the target nucleic acid is a viral RNA structure or genetic element rarely found in mammalian genomes or exclusively found in viral genomes. In some embodiments, the viral RNA structure or genetic element is an IRES element.

The disclosed compounds and methods may be used to target virtually any virus, because every virus must produce RNA and thence proteins at some point in their life cycles. In some embodiments the virus is selected from a Group I (dsDNA viruses), Group II (ssDNA viruses), Group III (dsRNA viruses), Group IV ((+)sense RNA viruses), Group V ((−) sense RNA viruses), Group VI (RNA reverse transcribing viruses), Group VII (DNA reverse transcribing viruses) virus, or a subviral agent such as a satellite or viroid.

For additional target viruses, see, e.g. http://www.virology.net/Big_Virology/BVFamilyGroup.html. In addition to targeting viral RNA transcripts containing a 3WJ, mixed RNA/DNA hybrids comprising viral nucleic acids may be targeted. For example, viral genomic DNA interacts with endogenous RNA effectors to produce 3WJs from mixed RNA/DNA hybridization events. Accordingly, in some embodiments the target nucleic acid is a mixed RNA/DNA hybrid comprising genetic information from a virus such as those disclosed above, wherein the hybrid is capable of forming or contains one or more 3WJs.

In some embodiments, the virus is an RNA virus (i.e., a virus having an RNA genome) such as a flavivirus, for example Zika virus, West Nile virus, Dengue virus, Yellow Fever virus, or Japanese encephalitis (Shi (editor), Molecular Virology and Control of Flaviviruses, 2012, Caister Academic Press). In some embodiments the virus is a coronavirus. Coronaviruses are a family of single-strand (ss) RNA viruses that includes human pathogens such as SARS-causing virus SARS-CoV (Thiel, (editor), Coronaviruses: Molecular and Cellular Biology (1st ed.), 2007, Caister Academic Press). Other RNA viruses that may be treated by the present invention include Ebola virus. In some embodiments, one or more of the foregoing RNA viruses is targeted by small molecule-induced 3WJ formation and/or small molecule-mediated stabilization of the 3WJ in order to block replication, translation or packaging of the viral RNA. In some embodiments, the target RNA is the RNA viral genome of the viral pathogen and the effector RNA is an endogenous small RNA in the infected cell.

In some embodiments, the virus is selected from a Group IV single-stranded RNA virus such as Coxsackie virus, Norovirus, Measles virus, Hepatitis C virus, Zika virus, Ebola virus, Rabies virus, West Nile virus, Dengue virus, SARS coronavirus, or Yellow fever virus. In some embodiments, the virus is selected from a Coronavirus such as Avian infectious bronchitis virus, Bovine coronavirus, Canine coronavirus, Feline infectious peritonitis virus, Human coronavirus 299E, Human coronavirus OC43, Murine hepatitis virus, Porcine epidemic diarrhea virus, Porcine hemagglutinating encephalomyelitis virus, Porcine transmissible gastroenteritis virus, Rat coronavirus, Turkey coronavirus, Rabbit coronavirus, Torovirus, Berne virus, or Breda virus. In some embodiments, the virus is selected from a Filovirus, Marburg virus, or Ebola virus.

### Microbial Nucleic Acid Targets

Various other infectious agents provide suitable target nucleic acids. In some embodiments, the target nucleic acid is a bacterial nucleic acid. Nucleic acids such as RNA in pathogenic bacteria provide target sequences that are critical for the life cycle of the pathogens or affect the bacterial life cycle, virulence, or pathogenicity and in some cases differ greatly in sequence and function from human RNAs. For example, the target bacteria may be a tuberculosis-causing bacteria.

### Fungal and Parasitic Targets

In some embodiments, the target nucleic acid is a fungal or parasitic nucleic acid, such as a malarial nucleic acid. Nucleic acids such as RNA in pathogenic fungi and parasites provide target sequences that are critical for the life cycle of the pathogens or affect the life cycle, virulence, or pathogenicity and in some cases differ greatly in sequence and function from human RNAs.

miRNA as Effectors

MicroRNA (miRNA) is a class of single-strand noncoding RNAs that are approximately 22 nucleotides (nt) in length (Friedman et al., Genome Res. 2009, 19, 92-105; Ghildiyal & Zamore, Nat. Rev. Genet. 2009, 10, 94-108; Ipsaro & Joshua-Tor, Nat. Struct. Mol. Biol. 2015, 22, 20-28; Izaurralde, Science 2015, 349, 380-382). They play important regulatory roles in repressing gene expression by functioning at the post-transcriptional level. They are well-conserved and are believed to be critical components in gene regulation across many species in plants and animals. Their “seed” sequence can interact with sequences in the 3′ untranslated region (UTR) of target mRNA, and less frequently, the 5′ end of mRNA. This base-pairing event leads to a decrease in the expression of the target gene by inducing degradation of the mRNA or by inhibiting translation of the mRNA into protein. miRNAs influence a wide range of biological processes including cell differentiation, cell fate, motility, survival, and cell function. miRNAs can be associated with a variety of human diseases including cancer as well as metabolic, cardiovascular, neurological and inflammatory diseases (Croce, Nature Rev. Genet. 2009, 10, 704-714; Dykxhoorn Cancer Res. 2010, 70, 6401-6406; Quiant & Olson, J. Clin. Invest. 2013, 123, 11-18; Olson, Science Trans. Med. 2014, 6, 239ps3; Baffy, J. Clin. Med. 2015, 4, 1977-1988). In many settings, the alteration in miRNA expression is associated with human diseases.

Accordingly, in some embodiments the present invention provides a method of modulating the activity of a target miRNA or precursor or protein-bound complex or mRNA- or ncRNA-bound complex thereof, comprising contacting the target miRNA or precursor or protein-bound complex or mRNA- or ncRNA-bound complex thereof with a disclosed compound.

miRNAs are expressed from their own distinct genes or are expressed within introns, or in some cases exons, of other genes. Most miRNAs are transcribed by RNA polymerase II. The primary transcript containing the miRNA sequence is known as a pri-miRNA. Pri-miRNAs have unique structural features that differ from other types of RNA, including a segment that folds to form a “hairpin” or stem-loop structure that is flanked by segments of a single-strand RNA. This double-stranded RNA structure can be recognized by the microprocessor complex that contains the RNA-binding protein DGCR8 (DiGeorge Syndrome Critical Region 8) and the RNase III enzyme Drosha. DGCR8 orients the catalytic RNase III domain of Drosha to produce shorter hairpins from pri-miRNAs by cleaving RNA at approximately eleven nucleotides from the hairpin base (one helical double-strand RNA turn into the stem). The microprocessor complex cuts the pri-miRNA to generate a pre-miRNA of 60 to 70 nt. A single pri-miRNA can contain one to six pre-miRNAs. The pre-miRNA itself has a distinct stem-loop structure. It interacts with Exportin-5 and Ran GTPase, leading to its transport from the nucleus into the cytoplasm. In the cytosol, the miRNA-protein complex is recognized by the RNase III enzyme Dicer that cleaves the pre-miRNA into a mature miRNA. The Dicer endoribonuclease interacts with 5′ and 3′ ends of the hairpin and cleaves the loop joining the 3′ and 5′ arms, yielding a miRNA duplex. The final miRNA is an approximately 22 base pair duplex having 2 nt 3′ overhangs and 5′ phosphate groups. One strand of this mature miRNA is then loaded onto Argonaute (Ago) protein to form the RNA-Induced Silencing Complex (RISC). Members of the Ago protein family are central to RISC function. Ago proteins are needed for miRNA-induced silencing and contain two conserved RNA binding domains—a PAZ domain that can bind the single stranded 3′ end of the mature miRNA and a PIWI domain that structurally resembles ribonuclease H and functions to interact with the 5′ end of the guide strand of the miRNA. These proteins bind the mature miRNA and orient it for interaction with a target mRNA. Some Ago family members such as the human Ago2 cleave the RNA targets directly. Ago proteins also can recruit additional proteins to inhibit translation. Although either strand of the duplex may potentially act as a functional miRNA, only one strand is incorporated into the RISC complex with which the miRNA and its mRNA target ultimately interact. miRNA base pairs with its target mRNA, leading to decreased target mRNA levels or inhibition of its translation. Induction of mRNA cleavage leading to mRNA decay appears to be the more common mechanism of inhibition of expression. Translational repression is less well understood than the induction of mRNA degradation. As noted above, a single miRNA can have many target mRNAs. Besides targeting mRNA, miRNAs also can target noncoding RNAs.

Thus, in some embodiments, the target miRNA is a precursor to a corresponding mature miRNA. In some embodiments, the target miRNA is a pri-miRNA. In some embodiments, the target miRNA is a pre-miRNA. In some embodiments, the target miRNA is an mRNA- or ncRNA-bound complex such as a mature miRNA bound to an mRNA or ncRNA whose activity it regulates. In some embodiments, the target miRNA is associated with a disease or disorder such as cancer, a metabolic disorder, a cardiovascular disorder, a neurological disorder, or an inflammatory disease. In some embodiments, the compound up-regulates the activity of the target miRNA. In some embodiments, the compound down-regulates the activity of the target miRNA. In some embodiments, the down-regulation is via binding to a 3WJ in the target miRNA-mRNA or miRNA-ncRNA complex. In some embodiments, binding to the 3WJ inhibits translation of the mRNA. In some embodiments, binding to the 3WJ induces degradation of the mRNA or ncRNA.

Also provided is a method of producing a small molecule that modulates the activity of a target miRNA or precursor or protein-bound complex or mRNA- or ncRNA-bound complex thereof, comprising the steps of: screening one or more small molecules disclosed herein for binding to a 3WJ in the target miRNA or precursor or protein-bound complex or mRNA- or ncRNA-bound complex thereof; and identifying which small molecule(s) bind to the 3WJ and modulate the activity of the target miRNA or precursor or protein-bound complex or mRNA- or ncRNA-bound complex thereof. In some embodiments, the method further comprises identifying the target miRNA or precursor or protein-bound complex or mRNA- or ncRNA-bound complex thereof according to a disclosed computational survey. In some embodiments, the target miRNA is an effector capable of forming a 3WJ with an mRNA or ncRNA which it regulates. In some embodiments, the small molecule binds to a trans 3WJ formed between the effector and the mRNA or ncRNA. In some embodiments, the small molecule binds to a cis 3WJ formed by a portion of the mRNA or ncRNA.

There are roughly 2000 miRNAs in the human genome. miRNAs can be expressed in a tissue-specific and cell-specific manner. Their expression can be affected by a variety of stimuli including hormones, cell stress, oncoproteins, cytokines and hypoxia. As stated above, aberrant miRNA expression can be associated with a wide variety of human diseases, and this aberrant miRNA expression is correlated with the dysregulation of the expression of their target genes.

Perfect base-pairing of the entire miRNA with target mRNA is rare in mammalian cells. It is believed that perfect or near perfect base pairing with the target mRNA promotes cleavage of the target RNA. A short stretch of nt in the 5′ end of the miRNA (“the seed”) appears to be sufficient for base-pairing to the target sequence in the mRNA. This base-pairing can comprise as few as 6 nt. The limited sequence complementarity needed for miRNA-mRNA interaction enables single miRNAs to regulate a large number of transcripts. It has been documented that individual miRNA can affect the expression of well over 100 different mRNA, with estimates as high as 400 mRNA targets. Although the requirements for miRNA recognition of mRNA do not appear to be stringent, research has led to a description of rules that predict canonical miRNA targeting. Seed nucleotides 2-8 at the 5′ end of the miRNA are important for target recognition. Crystal structures of the Ago-miRNA complex (Schirle et al., Science 2014, 346, 608-613) suggest that the seed sequence is positioned to initiate the interaction between the miRNA and its target mRNA. A perfect seed complementarity is considered to be canonical targeting, but imperfect or non-seed interactions are observed (reviewed in Seok et al., Mol. Cell, 2016, 39, 375-381). Thus, it appears that a variety of base-pairing can be used to achieve miRNA-mRNA recognition. Non-canonical pairing can take place and may have biological consequences. It has been hypothesized that these weaker, non-canonical, binding events could lead to less effective repression of expression and could, therefore, provide for a range of effectiveness in the repressive activity of miRNA. It even has been reported that human microRNA miR369-3 directs the association of Ago and FXR1 proteins with sequences in the 3′ UTRs of mRNA to increase translation, while another miRNA, Let-7, also can increase translation of target mRNAs upon cell cycle arrest (Vasudevan et al., Science 2007, 318, 1931-1934). Therefore, it is demonstrated that miRNAs can have a wide range of effects in mammalian cells. Accordingly, in some embodiments, a disclosed compound binds to a 3WJ formed between a target miRNA and an mRNA or ncRNA that the miRNA regulates. In some embodiments, a sequence of nt in the 5′ end of the miRNA (the seed) is capable of base-pairing to a target sequence in the mRNA. In some embodiments, a sequence of nt in the 3′ end of the miRNA (the seed) is capable of base-pairing to a target sequence in the mRNA. In some embodiments the base-pairing comprises canonical (Watson-Crick) base-pairing, for example between 75%; 80%; 85%; 90%; 95%; 98%; 99%; or all of the seed and its target sequence. In some embodiments, the base-pairing comprises at least 6 nt. In some embodiments, the base-pairing comprises at least 7, 8, 9, 10, 11, 12, 13, 14, 15, or 20 nt. In some embodiments, the base-pairing comprises at least 25, 30, 35, 40, 45, 50, 75, or 100 nt.

The rapid and controlled turnover of mature miRNA is needed for rapid changes in miRNA expression profiles and resulting gene regulation. For example, the binding of miRNA to Ago protein in the cytoplasm is believed to stabilize the miRNA guide strand, while the opposite passenger strand is degraded. Target engagement also may stabilize the miRNA. Post-transcriptional modifications of the miRNA also can affect their half-lives in cells.

In some embodiments, the miRNA is an effector such that the miRNA recognizes a target sequence in an RNA such as an mRNA and bind in a manner which leads to the formation of a 3WJ. A described above, in some embodiments this 3WJ comprises a stem-loop structure flanked on each side by sequences of base pairing between the miRNA and mRNA. The stem-loop can be formed by either the miRNA or mRNA. In some embodiments, a stable 3WJ is formed and degradation of the mRNA is not promoted. In some embodiments, there is not perfect complementarity (canonical base-pairing) between the miRNA seed (miRNA positions 2-7 or 2-8 from the 5′ end) and the target mRNA. In some embodiments, the base pairing is just downstream of the seed or can overlap with the seed, but does not include the entire seed. For example, nt 5-10 of the miRNA could have perfect base pairing with the mRNA target, immediately followed by a formation of a stem-loop by the mRNA sequence, and then the miRNA could base pair perfectly with the mRNA over a stretch of 4-12 nt starting at miRNA nt 11, 12, 13 or 14.

piRNA as Effectors

Piwi-interacting RNA (piRNA) is the largest class of small noncoding RNA expressed in animal cells (reviewed in Seto et al., Molecular Cell, 2007, 26, 603-609; Klattenhoff and Theurkauf, Development 2008, 135, 3-9, 2008). There may be 100,000 or more piRNAs in animal cells. These RNAs are 26-31 nt in length. piRNAs derive their name due to their interaction with piwi proteins. They are reported to be involved in gene silencing, but far less is known about the generation of piRNA and their mechanism of gene silencing relative to miRNA. Many piRNAs are antisense to transposons; therefore, the silencing of transposons may be a critical role of piRNAs. They are found to localize to both nuclear and cytosolic compartments of the cell. piRNAs are abundant in germ cells where they may play critical roles in germ cell development and function. However, they also are abundant in a wide variety of mammalian somatic cells and tissues.

Accordingly, in some embodiments the present invention provides a method of modulating the activity of a target piRNA or precursor or protein-bound complex thereof, comprising contacting the target piRNA or precursor or protein-bound complex thereof with a disclosed compound.

In some embodiments, the target piRNA is associated with a disease or disorder such as a disease involving aberrant germ cell development or function. In some embodiments, the compound up-regulates the activity of the target piRNA. In some embodiments, the compound down-regulates the activity of the target piRNA. In some embodiments, the down-regulation is via binding to a 3WJ in the target piRNA or precursor or protein-bound complex thereof. In some embodiments, binding to the 3WJ induces degradation of the piRNA.

Also provided is a method of producing a small molecule that modulates the activity of a target piRNA or precursor or protein-bound complex thereof, comprising the steps of: screening one or more small molecules disclosed herein for binding to a 3WJ in the target piRNA or precursor or protein-bound complex thereof; and identifying which small molecule(s) bind to the 3WJ and modulate the activity of the target piRNA or precursor or protein-bound complex thereof In some embodiments, the method further comprises identifying the target piRNA or precursor or protein-bound complex thereof according to a disclosed computational survey. In some embodiments, the target piRNA is an effector capable of forming a 3WJ with an RNA which it regulates. In some embodiments, the small molecule binds to a trans 3WJ formed between the effector and the RNA which it regulates. In some embodiments, the small molecule binds to a cis 3WJ formed by a portion of the piRNA.

snoRNA as Effectors

Small nucleolar RNAs (snoRNAs) are small RNAs that direct chemical modifications such as methylation or pseudouridylation of other RNAs such as ribosomal RNAs, tRNAs, and small nuclear RNAs (snRNA) (reviewed in Bachellerie et al., Biochimie 2002, 84, 775-790; Jorjani et al., Nucleic Acids Res. 2016, May 12 [Epub ahead of print]). They are typically 70-90 nt in length. 1740 snoRNAs have been identified. They are fairly abundant in mammalian cells. To carry out RNA modifications, snoRNAs associate with proteins in a small nucleolar ribonucleoprotein (snoRNP) complex. The snoRNA contains an antisense sequence of 10-20 nt complementary to the sequence surrounding the base targeted for modification. Most snoRNAs are encoded in the introns of genes encoding proteins involved in ribosome synthesis or translation. They also can be located in intergenic regions, ORFs of protein coding genes, and in UTRs. They can be transcribed by either RNA polymerase II or III. The sequences of snoRNA are conserved, but their expression levels differ dramatically between species.

Accordingly, in some embodiments the present invention provides a method of modulating the activity of a target snoRNA or precursor or protein-bound (such as a snoRNP) complex thereof, comprising contacting the target snoRNA or precursor or protein-bound complex thereof with a disclosed compound.

In some embodiments, the target snoRNA is associated with a disease or disorder such as a disease involving aberrant nucleic acid modification such as methylation or pseudouridylation. In some embodiments, the aberrantly modified nucleic acid is a ribosomal RNA, a tRNA, or an snRNA. In some embodiments, the compound up-regulates the activity of the target snoRNA. In some embodiments, the compound down-regulates the activity of the target snoRNA. In some embodiments, the down-regulation is via binding to a 3WJ in the target snoRNA or precursor or protein-bound complex thereof.

Also provided is a method of producing a small molecule that modulates the activity of a target snoRNA or precursor or protein-bound (such as a snoRNP) complex thereof, comprising the steps of: screening one or more small molecules disclosed herein for binding to a 3WJ in the target snoRNA or precursor or protein-bound (such as a snoRNP) complex thereof; and identifying which small molecule(s) bind to the 3WJ and modulate the activity of the target snoRNA or precursor or protein-bound complex thereof. In some embodiments, the method further comprises identifying the target snoRNA or precursor or protein-bound complex thereof according to a disclosed computational survey. In some embodiments, the target snoRNA is an effector capable of forming a 3WJ with an RNA which it regulates. In some embodiments, the small molecule binds to a trans 3WJ formed between the effector and the RNA which it regulates. In some embodiments, the small molecule binds to a cis 3WJ formed by a portion of the snoRNA.

### 3. Methods of Identifying Target 3 WJs

### Computational Survey of Potential Effector/Target Nucleic Acid Interactions

The present invention provides methods of identifying a potential target 3WJ in a nucleic acid. Information about nucleic acid three-dimensional structure is more challenging to obtain than proteins, and there exists a need for better methods for interrogating nucleic acid structure, particularly in vivo. Accordingly, in one aspect the present invention provides a method of identifying a target nucleic acid capable of forming a cis or trans 3WJ, wherein binding by a disclosed compound stabilizes the 3WJ, thus modulating the activity of the nucleic acid, for example to treat a disclosed disease or condition. In some embodiments, a provided method comprises as a first step running an in silico search that identifies potential interactions between effector RNAs and candidate target RNAs. In some embodiments, the search focuses on a target RNA selected based on prior knowledge of the value of targeting either that RNA itself or the protein that it expresses upon translation. The output of the search is a list of effector RNAs (such as small effector RNAs of, for example, 20-100 nt) that can hybridize with the target RNA such that a 3WJ can form. In other embodiments, a broader or even comprehensive, in silico search of all potential interactions between RNAs (as effectors) and all target RNAs in the cell is run. In some embodiments, the search is conducted initially without reference to any extant body of knowledge of the actual, known biological functions of the effector RNAs. Rather, each effector RNA is treated essentially as a chemical without reference to its biological role.

In one aspect, the present invention provides a method of identifying a target nucleic acid capable of forming a 3WJ comprising the following steps:
(a) providing one or more effector nucleic acids (such as a human small RNA) comprising one or more energetically accessible stem-loop structures;
(b) providing one or more target nucleic acids (such as a human mRNA) comprising one or more energetically accessible stem-loop structures;
(c) screening for one or more effector/target nucleic acid hybridization interactions that accommodate a stem-loop structure such as a 3WJ in either the effector nucleic acid or the target nucleic acid;
(d) optionally, categorizing the resulting database of 3WJs comprising an effector nucleic acid and target nucleic acid by one or more of: (1) loop topology, (2) loop sequence, (3) stem-loop stability, and (4) identity of the nucleobases that impinge directly on the 3WJ cavity; and
(e) optionally, cross-referencing the resulting database of possible 3WJs comprising an effector nucleic acid and target nucleic acid with available knowledge about the biological role and therapeutic significance of the target nucleic acids as well as available knowledge about the relative abundance and cellular location of effector nucleic acids such as small effector RNAs.

In step (a) above, in the case of effector miRNAs, in some embodiments a publicly available database is used as a source of potential effector miRNAs. In some embodiments, the database is selected from: (http://www.mirbase.org; http://mirdb.org/miRDB/; http.//www.microma.org/microma/home.do; http://mirtarbase.mbc.nctu.edu.tw; http://mircancer.ecu.edu; http://www.mir2disease.org; http://zmf.umm.uni-heidelberg.de/apps/zmf/mirwalk2/; or http://www.targetscan.org). In some embodiments, the target nucleic acid is human. In some embodiments, the target nucleic acid is viral. In some embodiments, a database of viral genomes is used, such as that available at http://www.ncbi.nlm.nih.gov/genome/viruses/. In some embodiments, the target nucleic acid is microbial. In some embodiments, the target nucleic acid is fungal. In some embodiments, the target nucleic acid is parasitic.

In step (a) above, in some embodiments, “energetically accessible” means that at least 0.1% of that miRNA in a living cell adopts (or is predicted to adopt) a given stem-loop structure. In other embodiments, the threshold of “accessible” might be set at 0.1% to 1%, 1% to 10%, or about 1%, 2%, 3%, 4%, 5%, 6%, 7%, 8%, 9%, or 10%. In some embodiments, to be included in the method above, such stem-loop needs to leave at least 4 nucleotides upstream (5′) and at least 4 nucleotides downstream (3′) of the stem-loop that can participate in hybridization with the target nucleic acid.

In step (b) above, in some embodiments, “energetically accessible” means that at least 0.1% of that miRNA in a living cell adopts (or is predicted to adopt) a given stem-loop structure. In other embodiments, the threshold of “accessible” might be set at 0.1% to 1%, 1% to 10%, or about 1%, 2%, 3%, 4%, 5%, 6%, 7%, 8%, 9%, or 10%. In some embodiments, to be included in the method above, such stem-loop needs to leave at least 4 nucleotides upstream (5′) and at least 4 nucleotides downstream (3′) of the stem-loop that can participate in hybridization with the target nucleic acid.

In step (c) above, in some embodiments, the method produces a compilation of all effector/target nucleic acid pairs that create a 3WJ satisfying the constraints described above. In all three of the possible unhybridized inter-stem loops in a 3WJ, the lengths of those loops can vary independently from 0 to 4 (or, in some embodiments, 0 to 2 or 0 to 1), with any of the standard or post-transcriptionally modified nucleobases occupying those loop positions.

In some embodiments, pairings that engage therapeutically high-value target nucleic acids with relatively high-abundance small effector RNAs will be the focus of further study. The structure, dynamics, and biological significance of the selected key 3WJs can be characterized extensively using a wide range of established methods described below.

In some embodiments, a 3WJ such as a 3WJ identified in the above computational screening methods, may be characterized by synthesizing the effector nucleic acid (such as a small effector RNA) and its cognate target nucleic acid using standard, commercially available, machine-enabled synthetic methods. In addition, in many embodiments, a single-stranded (“cis”) analog of a trans-3WJ is synthesized; in such embodiments, the two components can be linked either 5′-to-3′ or 3′-to-5′. The advantages of a single-stranded model include simplified assay formats and controlled stoichiometry in structural characterization.

Whether in trans or in cis, the model effector/target nucleic acid interacting pairs can be structurally characterized in detail using x-ray crystallography, cryo-EM (Binshtein & Ohi, Biochemistry 2015, 54, 3133-3141), NMR, circular dichroism, UV-vis spectroscopy, and SHAPE-MaP (Siegfried et al., Nature Methods 2014, 11, 959-973), and related methods that help to establish the detailed 3D structure and dynamics of the construct and thus, by implication, allow inference of the structure and dynamics of the congeneric trans pair in cells.

### Building Binding Assays to Measure SM Binding to 3WJs

One of the principal purposes in building the above cis and trans models is to screen for small molecules that will bind selectively and with high affinity into the cavities defined by the 3WJs. Many methods that are currently deployed for analogous screening of small molecules against protein targets can be adapted to similar effect in 3WJs. In some embodiments, one of the following exemplary assays is used to measure small molecule binding to an effector/target nucleic acid 3WJ:

NMR—NMR can be used to assess the binding of small molecules to biomolecules such as RNA. One key advantage of this method is that, if the structure of the target biomolecule can be observed during the screen, then the screen will yield not just the fact of molecular association between SM and RNA, but also the subsite and binding mode. Some of the disadvantages of NMR are that relatively high concentrations of ligand are required (μM to mM), meaning that high-affinity interactions are difficult to measure, and that the method is slow, thus permitting only limited throughput. But these latter problems actually make NMR an ideal read-out in carrying out fragment screening, where high-concentration/low-affinity interactions are the focus and relatively few fragments (typically <5000) are usually screened in fragment-based lead discovery. In some cases the atomic nucleus that is observed is ¹⁹F rather than protons.

SPR—Surface plasmon resonance is a widely implemented method for assessing molecular associations. SPR requires that one component (the target RNA or the small molecule ligand) be covalently affixed to the surface and the plasmon resonance is measured in response to partner molecules added to the solution above the immobilized partner. On rates and off rates can be measured directly, affording equilibrium dissociation constants.

DELs—DNA-encoded libraries serve here as an example of a broader category of pull-down methods. In DELs, a cis model of a key 3WJ would be modified with functional groups (e.g., biotin) that allowed facile pull-down (e.g., with streptavidin) after having been exposed to a homogeneous DNA-encoded library. Those small molecule ligands in the DEL that associate tightly with the 3WJ will also be pulled down when the 3WJ is pulled down and the identity of the ligands exposed using PCR and sequencing.

Chromophoric 3WJs—There are multiple reports of derivatizing RNAs such that two chromophores interact when proximal, leading to either activation or suppression of emission, which in turn relies upon the RNA assuming the correct conformation (in this case the anticipated 3WJ). Loop sequence, loop length, temperature, and other parameters can be optimized so that achieving the 3WJ conformation depends on the stabilizing influence of an added small molecule that binds into the 3WJ cavity. These assays are straightforward and can be run in heterogeneous or homogeneous formats and allow screening of standard, commercially available libraries of small molecules.

RNA Microarrays—One can immobilize a wide range of 3WJs onto a microarray—in effect an immobilized library of targets. The chromophore functionality described above can be incorporated so that when the surface-immobilized library of RNA (or other nucleic acid) targets is exposed to small molecules (singly or as mixtures), chromophoric change at specific sites on the microarray indicate that that RNA (or other nucleic acid) is ligated and, more importantly, stabilized in the 3WJ conformation.

SM Microarrays—One can immobilize a wide range of small molecules onto a microarray. There is a broad literature on this topic and a commensurately broad range of immobilization techniques. In such microarrays, the identity of the SM is typically associated with its position in the microarray. Chromophoric 3WJ models can then be exposed in to the microarray and specific SM/3WJ interactions identified by the observation of the chromophore at specific sites on the microarray.

MS—Nucleic acids can be analyzed by a variety of liquid chromatography techniques. Chromatography of RNA in the presence of potential small molecule ligands would lead to bound small molecule ligands being carried with the RNA as it passes through the chromatography media. Post-chromatographic separation of the ligand from the RNA will allow identification of the binding ligands by mass spectrometry. If two ligands share the same mass (are isomers), there are numerous pathways to disambiguation.

Initial Screens of 3WJs—Initial screening of small molecules against the 3WJ models will focus on three types of libraries: (1) Compounds that are C3-symmetric or are variations on C3-symmetric scaffolds; (2) Compounds that are variations on reduced-symmetry scaffolds that still approximate the C3-symmetric congeners; and (3) A structurally diverse library. These scaffolds and exemplary embodiments thereof are described in detail below.

Structural Characterization of 3WJ/SM Complexes—Many therapeutically interesting RNA targets are large and only modestly structured, so detailed structural characterization is either not possible or not practical. However, targeting of 3WJs as in the present invention means that the target substructure modeled by the synthetic 3WJ is relatively small and its complexes with SMs may be characterized in detail. Methods of structural characterization include NMR, x-ray crystallography, cryo-EM, and CD. Such a detailed characterization of a small molecule complexed with its target RNA provides nearly unprecedented opportunities for structure-based drug design against an RNA target.

### Confirmation of Trans Effect

In much of the foregoing the focus is on the cis (single-stranded) models. When the therapeutic targets are composed of a trans interaction between an effector RNA and a target RNA, then the single-stranded cis construct is studied out of convenience and any small molecule ligand needs to be demonstrated to bind to and stabilize the congeneric trans complex upon which the cis model is based. Put differently, it is essential that the selected small molecules identified via design or screening induce the formation of the intended effector/SM/target ternary complex.

### Building Competitive Displacement Assays

The screens described above will identify small molecule leads against trans effector/target nucleic acid 3WJs of interest. While exploration of the SAR from those leads to development candidates may be possible using the methods and techniques described above, it is often important to develop assays where new molecules compete with a reference molecule for the same biologically essential subsite, in this case the cavity circumscribed by the 3WJ. This assay format is often very high-throughput and focuses the screen to find molecules that share the same binding mode as the reference molecule. In some embodiments, such an assay comprises the steps of: (1) identification of the initial small molecule for testing, described above and below, (2) modification of that small molecule, where necessary, to include a chromophoric read-out where displacement either activates or suppresses emission and (3) concomitant modification, where necessary, of the nucleic acid target proximal to the binding site to incorporate a complementary chromophoric read-out.

### Demonstration of Cellular Activity

All of the foregoing takes place in relatively artificial biochemical settings. But the selected small molecules need to induce the formation of the ternary complex inside cells. Furthermore, the formation of that ternary complex needs to impact the stability, function, and/or translation of the targeted nucleic acid, e.g. RNA, which will in some embodiments is a pre-mRNA or mRNA. The demonstration that the required ternary complex as formed inside the cell can be readily achieved by making a tethered reagent, based on the indicated ligand, and performing PEARL-seq or other affinity-based techniques on that ternary complex.

Impacting the functional career of a targeted RNA can be demonstrating using standard reporter gene assay methods. In some embodiments, the target nucleic acid, e.g. RNA sequence, is introduced into a Luciferase reporter vector or other standard reporter construct and the impact of the 3WJ and small molecule on the reporter expression is measured in cells. In cases in which the 3WJ and small molecule will affect the expression levels of the target, the levels of the RNA can be measured by quantitative RT-PCR and the level of protein measured by ELISA, Western blot or FRET assay.

### 2. Compounds and Embodiments Thereof

In one aspect, the present invention provides compounds, and pharmaceutically acceptable compositions thereof, for use in modulating the activity of a target nucleic acid by binding to a 3WJ in the target nucleic acid or an effector/nucleic acid complex. Such compounds are effective for treating, preventing, or ameliorating a disease or condition associated with a target RNA; and for use in methods described herein.

Compounds that may be used in the present invention and methods of discovering such compounds (such as SHAPE-MaP, RING-MaP, and PEARL-seg™ (also known as Hook the Worm and Hook and Click methods) are described in U.S. Provisional Patent Application U.S. Ser. No. 62/289,671, which is hereby incorporated by reference in its entirety.

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M.B. and March, J., John Wiley & Sons, New York: 2001, the entire contents of which are hereby incorporated by reference.

### Small Molecules

The design and synthesis of novel, small molecule ligands capable of binding RNA represents largely untapped therapeutic potential. Certain small molecule ligands including macrolides (e.g., erythromycin, azithromycin), alkaloids (e.g., berberine, palmatine), aminoglycosides (e.g., paromomycin, neomycin B, kanamycin A), tetracyclines (e.g., doxycycline, oxytetracycline), theophyllines, and oxazolidinones (e.g., linezolid, tedizolid) are known to bind to RNA, paving the way for the search for small molecules as RNA targeting drugs. Organic dyes, amino acids, biological cofactors, metal complexes as well as peptides also show RNA binding ability. It is possible to modulate RNAs such as riboswitches, RNA molecules with expanded nucleotide repeats, and viral RNA elements.

The terms “small molecule that binds a target RNA,” “small molecule RNA binder,” “affinity moiety,” or “ligand moiety,” as used herein, include all compounds generally classified as small molecules that are capable of binding to a target RNA with sufficient affinity and specificity for use in a disclosed method, or to treat, prevent, or ameliorate a disease associated with the target RNA. Small molecules that bind RNA for use in the present invention may bind to one or more secondary or tertiary structure elements of a target RNA. These sites include RNA triplexes, 3WJs, 4WJs, parallel-Y junctions, hairpins, bulge loops, pseudoknots, internal loops, and other higher-order RNA structural motifs described or referred to herein.

Accordingly, in some embodiments, the small molecule that binds to a target RNA is selected from a macrolide, alkaloid, aminoglycoside, a member of the tetracycline family, an oxazolidinone, an SMN2 ligand, ribocil or a related compound, an anthracene, or a triptycene. In some embodiments, the small molecule RNA binder is selected from paromomycin, a neomycin (such as neomycin B), a kanamycin (such as kanamycin A), linezolid, tedizolid, pleuromutilin, ribocil, NVS-SM1, anthracene, or triptycene. In some embodiments, the small molecule is selected from those shown in U.S. Provisional Patent Application U.S. Ser. No. 62/289,671, which is hereby incorporated by reference in its entirety; or a pharmaceutically acceptable salt, stereoisomer, or tautomer thereof. Further exemplary small molecules are described in detail below.

In some embodiments, the present invention provides a compound of Formula I:

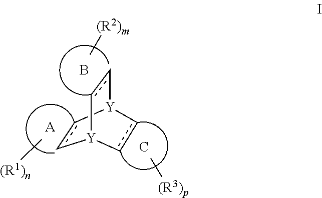

or a pharmaceutically acceptable salt thereof, wherein:
Rings A, B, and C are each, independently, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each Y is independently CR or N,
each R¹ is independently —R, halogen, —CN, —OR, —N(R)₂, —NO₂, —N₃, —SR, or —L¹—R⁶;
each R² is independently —R, halogen, —CN, —OR, —N(R)₂, —NO₂, —N₃, —SR, —L²—R⁶, or two R² groups on the same carbon are optionally taken together to form ═NR⁶, ═NOR⁶, ═O, or ═S;
each R³ is independently —R, halogen, —CN, —OR, —N(R)₂, —NO₂, —N₃, —SR, or —L³—R⁶;
each R⁶ is independently hydrogen or Cl₁₋₆ alkyl optionally substituted with 1, 2, 3, 4, 5, or 6 halogens;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each L¹, L², and L³ is independently a covalent bond or a C₁₋₈ bivalent straight or branched hydrocarbon chain wherein 1, 2, or 3 methylene units of the chain are independently and optionally replaced with —O-, —C(O)-, —C(O)O-, —OC(O)-, —N(R)-, —C(O)N(R)-, —(R)NC(O)-, —OC(O)N(R)-, —(R)NC(O)O-, —N(R)C(O)N(R)-, —S-, —SO-, —SO₂-, —SO₂N(R)-, —(R)NSO₂-, —C(S)-, —C(S)O-, —OC(S)-, —C(S)N(R)-, —(R)NC(S)-, —(R)NC(S)N(R)-, or —Cy-;
m is 0, 1, 2, 3, or 4;
n is 0, 1, 2, 3, or 4; and
p is 0, 1, 2, 3, or 4.

As defined generally above, Rings A, B, and C are each, independently, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

In some embodiments, Ring A is a 3-8 membered saturated or partially unsaturated monocyclic carbocyclic ring. In some embodiments, Ring A is phenyl. In some embodiments, Ring A is an 8-10 membered bicyclic aromatic carbocyclic ring. In some embodiments, Ring A is a 4-8 membered saturated or partially unsaturated monocyclic heterocyclic ring having 1-2 heteroatoms independently selected from nitrogen, oxygen, or sulfur. In some embodiments, Ring A is a 5-6 membered monocyclic heteroaromatic ring having 1-4 heteroatoms independently selected from nitrogen, oxygen, or sulfur. In some embodiments, Ring A is an 8-10 membered bicyclic heteroaromatic ring having 1-5 heteroatoms independently selected from nitrogen, oxygen, or sulfur.

In some embodiments, Ring A is a 5-6 membered monocyclic heteroaromatic ring having 1-2 heteroatoms independently selected from nitrogen, oxygen, or sulfur.

In some embodiments, Ring B is a 3-8 membered saturated or partially unsaturated monocyclic carbocyclic ring. In some embodiments, Ring B is phenyl. In some embodiments, Ring B is an 8-10 membered bicyclic aromatic carbocyclic ring. In some embodiments, Ring B is a 4-8 membered saturated or partially unsaturated monocyclic heterocyclic ring having 1-2 heteroatoms independently selected from nitrogen, oxygen, or sulfur. In some embodiments, Ring B is a 5-6 membered monocyclic heteroaromatic ring having 1-4 heteroatoms independently selected from nitrogen, oxygen, or sulfur. In some embodiments, Ring B is an 8-10 membered bicyclic heteroaromatic ring having 1-5 heteroatoms independently selected from nitrogen, oxygen, or sulfur.

In some embodiments, Ring B is a 5-6 membered monocyclic heteroaromatic ring having 1-2 heteroatoms independently selected from nitrogen, oxygen, or sulfur.

In some embodiments, Ring B is absent.

In some embodiments, Ring C is a 3-8 membered saturated or partially unsaturated monocyclic carbocyclic ring. In some embodiments, Ring C is phenyl. In some embodiments, Ring C is an 8-10 membered bicyclic aromatic carbocyclic ring. In some embodiments, Ring C is a 4-8 membered saturated or partially unsaturated monocyclic heterocyclic ring having 1-2 heteroatoms independently selected from nitrogen, oxygen, or sulfur. In some embodiments, Ring C is a 5-6 membered monocyclic heteroaromatic ring having 1-4 heteroatoms independently selected from nitrogen, oxygen, or sulfur. In some embodiments, Ring C is an 8-10 membered bicyclic heteroaromatic ring having 1-5 heteroatoms independently selected from nitrogen, oxygen, or sulfur.

In some embodiments, Ring C is a 5-6 membered monocyclic heteroaromatic ring having 1-2 heteroatoms independently selected from nitrogen, oxygen, or sulfur.

In some embodiments, Rings A, B, and C are each, independently, selected from:

phenyl,

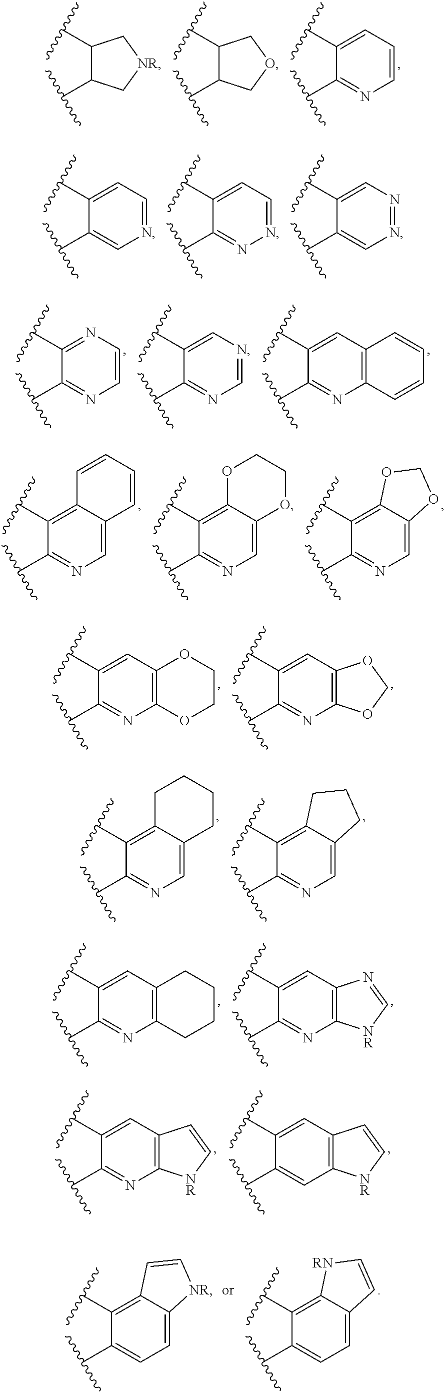

In some embodiments, Rings A, B, and C are each, independently, selected from:

phenyl,

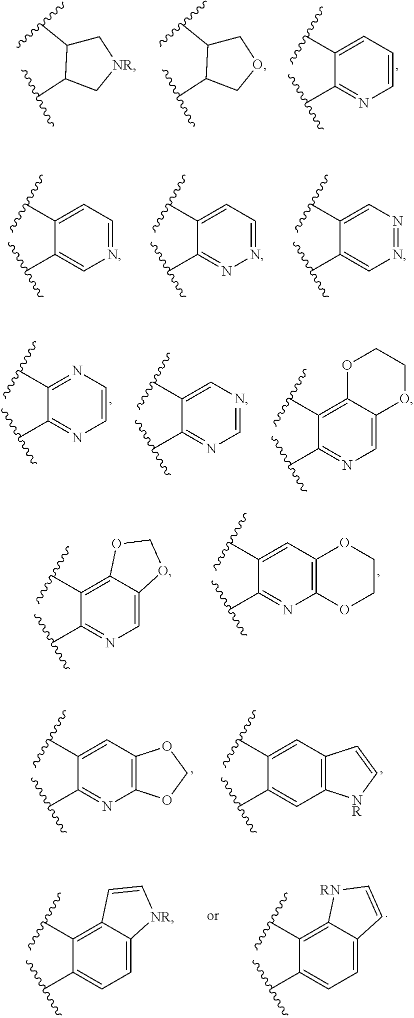

In some embodiments, Ring A is selected from:

phenyl,

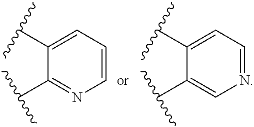 [SMILES: *c1cccnc1* ; *c1ccncc1*]

In some embodiments, Ring B is selected from:

phenyl,

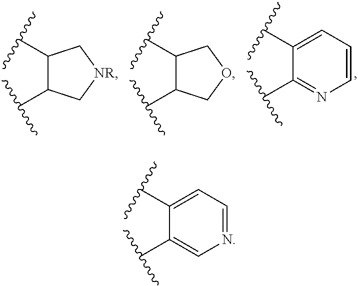

In some embodiments, Ring C selected from: phenyl,

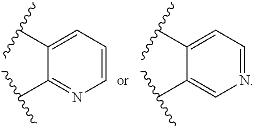 [SMILES: *c1cccnc1* ; *c1ccncc1*]

In some embodiments, at least one of Ring A, B, or C is

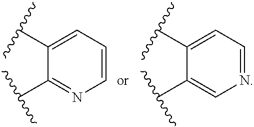 [SMILES: *c1cccnc1* ; *c1ccncc1*]

As defined generally above, each R¹ is independently R, halogen, —CN, —OR, —N(R)₂, —NO₂, —N₃, —SR, or —L¹—R⁶.

In some embodiments, R¹ is R. In some embodiments, R¹ is halogen. In some embodiments, R¹ is —CN. In some embodiments, R¹ is —OR. In some embodiments, R¹ is —N(R)₂. In some embodiments, R¹ is —NO₂. In some embodiments, R¹ is —N₃. In some embodiments, le is —SR. In some embodiments, R¹ is —L¹-R⁶.

In some embodiments, R¹ is hydrogen. In some embodiments, R¹ is an optionally substituted C₁₋₆ aliphatic group. In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 4-8 membered saturated or partially unsaturated monocyclic heterocyclic ring having 1-2 heteroatoms independently selected from nitrogen, oxygen, or sulfur. In some embodiments, R¹ is an optionally substituted 5-6 membered monocyclic heteroaromatic ring having 1-4 heteroatoms independently selected from nitrogen, oxygen, or sulfur. In some embodiments, R¹ is an optionally substituted 8-10 membered bicyclic heteroaromatic ring having 1-5 heteroatoms independently selected from nitrogen, oxygen, or sulfur.

As defined generally above, each R² is independently R, halogen, —CN, —OR, —N(R)₂, —NO₂, —N₃, —SR, or —L²—R⁶.

In some embodiments, R² is R. In some embodiments, R² is halogen. In some embodiments, R² is —CN. In some embodiments, R² is —OR. In some embodiments, R² is —N(R)₂. In some embodiments, R² is —NO₂. In some embodiments, R² is —N₃. In some embodiments, R² is —SR. In some embodiments, R² is —L²—R⁶.

In some embodiments, R² is hydrogen. In some embodiments, R² is an optionally substituted C₁₋₆ aliphatic group. In some embodiments, R² is an optionally substituted 3-8 membered saturated or partially unsaturated monocyclic carbocyclic ring. In some embodiments, R² is an optionally substituted phenyl. In some embodiments, R² is an optionally substituted 8-10 membered bicyclic aromatic carbocyclic ring. In some embodiments, R² is an optionally substituted 4-8 membered saturated or partially unsaturated monocyclic heterocyclic ring having 1-2 heteroatoms independently selected from nitrogen, oxygen, or sulfur. In some embodiments, R¹ is an optionally substituted 5-6 membered monocyclic heteroaromatic ring having 1-4 heteroatoms independently selected from nitrogen, oxygen, or sulfur. In some embodiments, R² is an optionally substituted 8-10 membered bicyclic heteroaromatic ring having 1-5 heteroatoms independently selected from nitrogen, oxygen, or sulfur.

As defined generally above, each R³ is independently R, halogen, —CN, —OR, —N(R)₂, —NO₂, —N₃, —SR, or —L³—R⁶.

In some embodiments, R³ is R. In some embodiments, R³ is halogen. In some embodiments, R³ is —CN. In some embodiments, R³ is —OR. In some embodiments, R³ is —N(R)₂. In some embodiments, R³ is —NO₂. In some embodiments, R³ is —N₃. In some embodiments, R³ is —SR. In some embodiments, R³ is —L³—R⁶.

In some embodiments, R³ is hydrogen. In some embodiments, R³ is an optionally substituted C₁₋₆ aliphatic group. In some embodiments, R³ is an optionally substituted 3-8 membered saturated or partially unsaturated monocyclic carbocyclic ring. In some embodiments, R³ is an optionally substituted phenyl. In some embodiments, R³ is an optionally substituted 8-10 membered bicyclic aromatic carbocyclic ring. In some embodiments, R³ is an optionally substituted 4-8 membered saturated or partially unsaturated monocyclic heterocyclic ring having 1-2 heteroatoms independently selected from nitrogen, oxygen, or sulfur. In some embodiments, R³ is an optionally substituted 5-6 membered monocyclic heteroaromatic ring having 1-4 heteroatoms independently selected from nitrogen, oxygen, or sulfur. In some embodiments, R³ is an optionally substituted 8-10 membered bicyclic heteroaromatic ring having 1-5 heteroatoms independently selected from nitrogen, oxygen, or sulfur.

As defined generally above, each L¹, L², and L³ is independently a covalent bond or a C₁₋₈ bivalent straight or branched hydrocarbon chain wherein 1, 2, or 3 methylene units of the chain are independently and optionally replaced with —O-, —C(O)-, —C(O)O-, —OC(O)-, —N(R)-, —C(O)N(R)-, —(R)NC(O)-, —OC(O)N(R)-, —(R)NC(O)O-, —N(R)C(O)N(R)-, —S-, —SO-, —SO₂-, —SO₂N(R)-, —(R)NSO₂-, —C(S)-, —C(S)O-, —OC(S)-, —C(S)N(R)-, —(R)NC(S)-, —(R)NC(S)N(R)-, or —Cy-.

In some embodiments, L¹ is a covalent bond. In some embodiments, L¹ is a C₁₋₈ bivalent straight or branched hydrocarbon chain. In some embodiments, L¹ is a C₁₋₈ bivalent straight or branched hydrocarbon chain wherein 1, 2, or 3 methylene units of the chain are independently and optionally replaced with —O-, —C(O)-, —C(O)O-, —OC(O)-, —N(R)-, —C(O)N(R)-, —(R)NC(O)-, —OC(O)N(R)-, —(R)NC(O)O-, —N(R)C(O)N(R)-, —S-, —SO-, —SO₂-, —SO₂N(R)-, —(R)NSO₂-, —C(S)-, —C(S)O-, —OC(S)-, —C(S)N(R)-, —(R)NC(S)-, —(R)NC(S)N(R)-, or —Cy-.

In some embodiments, L¹ is a C₁₋₆ bivalent straight or branched hydrocarbon chain wherein 1, 2, or 3 methylene units of the chain are independently and optionally replaced with —O-, —C(O)-, —N(R)-, —S-, —SO-, —SO₂-, —SO₂N(R)-, —(R)NSO₂-, —C(S)-, or —Cy-, and each R is independently hydrogen, —CH₂-phenyl, phenyl, C₁₋₆ alkyl, cyclopropyl, cyclobutyl, cyclopentyl, cyclohexyl, —CH₂F, —CHF₂, —CF₃, —CH₂CHF₂, or —CH₂CF₃; or each R is independently hydrogen or methyl; or R is hydrogen.

In some embodiments, L² is a covalent bond. In some embodiments, L² is a C₁₋₈ bivalent straight or branched hydrocarbon chain. In some embodiments, L² is a C₁₋₈ bivalent straight or branched hydrocarbon chain wherein 1, 2, or 3 methylene units of the chain are independently and optionally replaced with —O-, —C(O)-, —C(O)O, —OC(O)-, —N(R)-, —C(O)N(R)-, —(R)NC(O)-, —OC(O)N(R)-, —(R)NC(O)O-, —N(R)C(O)N(R)-, —S-, —SO-, —SO₂-, —SO₂N(R)-, —(R)NSO₂-, —C(S)-, —C(S)O-, —OC(S)-, —C(S)N(R)-, —(R)NC(S)-, —(R)NC(S)N(R)-, or —Cy-.

In some embodiments, L² is a C₁₋₆ bivalent straight or branched hydrocarbon chain wherein 1, 2, or 3 methylene units of the chain are independently and optionally replaced with —O-, —C(O)-, —N(R)-, —S-, —SO-, —SO₂-, —SO₂N(R)-, —(R)NSO₂-, —C(S)-, or —Cy-, and each R is independently hydrogen, —CH₂-phenyl, phenyl, C₁₋₆ alkyl, cyclopropyl, cyclobutyl, cyclopentyl, cyclohexyl, —CH₂F, —CHF₂, —CF₃, —CH₂CHF₂, or —CH₂CF₃; or each R is independently hydrogen or methyl; or R is hydrogen.

In some embodiments, L³ is a covalent bond. In some embodiments, L³ is a C₁₋₈ bivalent straight or branched hydrocarbon chain. In some embodiments, L³ is a C₁₋₈ bivalent straight or branched hydrocarbon chain wherein 1, 2, or 3 methylene units of the chain are independently and optionally replaced with —O-, —C(O)-, —C(O)O-, —OC(O)-, —N(R)-, —C(O)N(R)-, —(R)NC(O)-, —OC(O)N(R)-, —(R)NC(O)O-, —N(R)C(O)N(R)-, —S-, —SO-, —SO₂-, —SO₂N(R)-, —(R)NSO₂-, —C(S)-, —C(S)O-, —OC(S)-, —C(S)N(R)-, —(R)NC(S)-, —(R)NC(S)N(R)-, or —Cy-.

In some embodiments, L³ is a C₁₋₆ bivalent straight or branched hydrocarbon chain wherein 1, 2, or 3 methylene units of the chain are independently and optionally replaced with —O-, —C(O)-, —N(R)-, —S-, —SO-, —SO₂-, —SO₂N(R)-, —(R)NSO₂-, —C(S)-, or —Cy-, and each R is independently hydrogen, —CH₂-phenyl, phenyl, C₁₋₆ alkyl, cyclopropyl, cyclobutyl, cyclopentyl, cyclohexyl, —CH₂F, —CHF₂, —CF₃, —CH₂CHF₂, or —CH₂CF₃; or each R is independently hydrogen or methyl; or R is hydrogen.

As defined generally above, each —Cy- is independently a bivalent optionally substituted 3-8 membered saturated or partially unsaturated monocyclic carbocyclic ring, optionally substituted phenylene, an optionally substituted 4-8 membered saturated or partially unsaturated monocyclic heterocyclic ring having 1-3 heteroatoms independently selected from nitrogen, oxygen, or sulfur, an optionally substituted 5-6 membered monocyclic heteroaromatic ring having 1-4 heteroatoms independently selected from nitrogen, oxygen, or sulfur, an optionally substituted 8-10 membered bicyclic or bridged bicyclic saturated or partially unsaturated heterocyclic ring having 1-5 heteroatoms independently selected from nitrogen, oxygen, or sulfur, or an optionally substituted 8-10 membered bicyclic or bridged bicyclic heteroaromatic ring having 1-5 heteroatoms independently selected from nitrogen, oxygen, or sulfur.

In some embodiments, —Cy- is a bivalent optionally substituted 3-8 membered saturated or partially unsaturated monocyclic carbocyclic ring. In some embodiments, —Cy- is an optionally substituted phenylene. In some embodiments, —Cy- is an optionally substituted 4-8 membered saturated or partially unsaturated monocyclic heterocyclic ring having 1-3 heteroatoms independently selected from nitrogen, oxygen, or sulfur. In some embodiments, —Cy- is an optionally substituted 5-6 membered monocyclic heteroaromatic ring having 1-4 heteroatoms independently selected from nitrogen, oxygen, or sulfur. In some embodiments, —Cy- is an optionally substituted 8-10 membered bicyclic or bridged bicyclic saturated or partially unsaturated heterocyclic ring having 1-5 heteroatoms independently selected from nitrogen, oxygen, or sulfur. In some embodiments, —Cy- is an optionally substituted 8-10 membered bicyclic or bridged bicyclic heteroaromatic ring having 1-5 heteroatoms independently selected from nitrogen, oxygen, or sulfur.

As defined generally above, each R⁶ is independently hydrogen or C₁₋₆ alkyl optionally substituted with 1, 2, 3, 4, 5, or 6 halogens.

As defined generally above, m is 0, 1, 2, 3, or 4. In some embodiments, m is 0. In some embodiments, m is 1. In some embodiments, m is 2. In some embodiments, m is 3. In some embodiments, m is 4. In some embodiments, m is 0, 1, 2, or 3. In some embodiments, m is 0, 1, or 2. In some embodiments, m is 1, 2, or 3.

As defined generally above, n is 0, 1, 2, 3, or 4. In some embodiments, n is 0. In some embodiments, n is 1. In some embodiments, n is 2. In some embodiments, n is 3. In some embodiments, n is 4. In some embodiments, n is 0, 1, 2, or 3. In some embodiments, n is 0, 1, or 2. In some embodiments, n is 1, 2, or 3.

As defined generally above, p is 0, 1, 2, 3, or 4. In some embodiments, p is 0. In some embodiments, p is 1. In some embodiments, p is 2. In some embodiments, p is 3. In some embodiments, p is 4. In some embodiments, p is 0, 1, 2, or 3. In some embodiments, p is 0, 1, or 2. In some embodiments, p is 1, 2, or 3

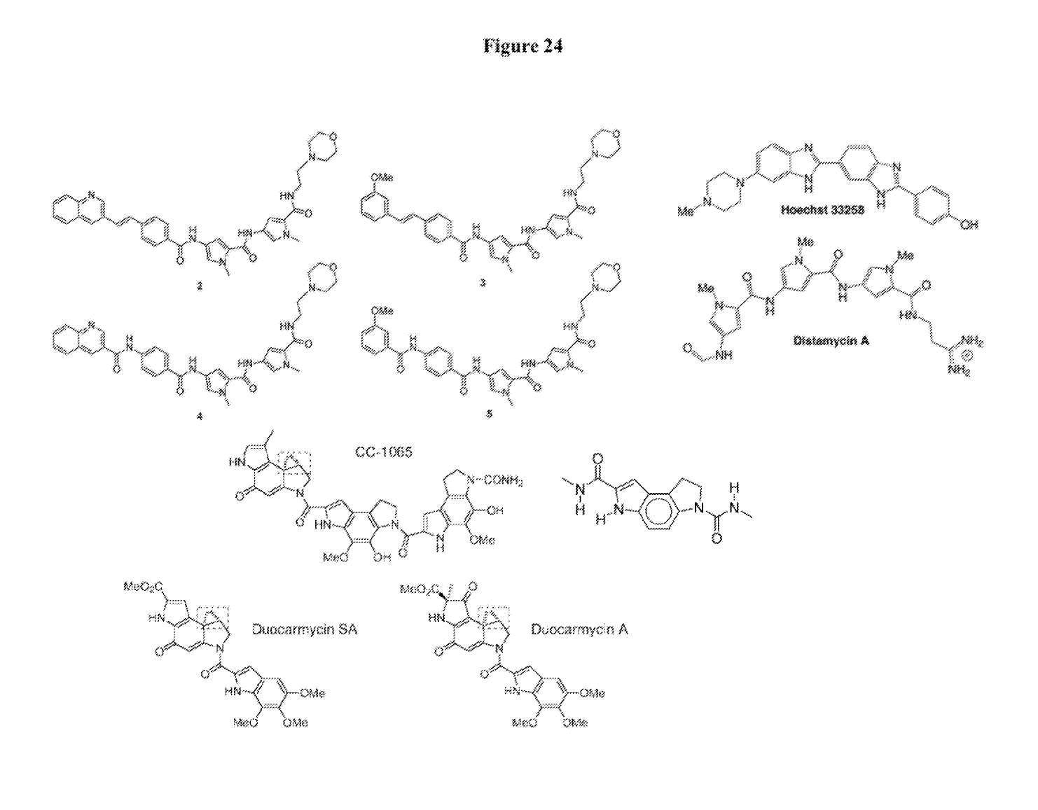

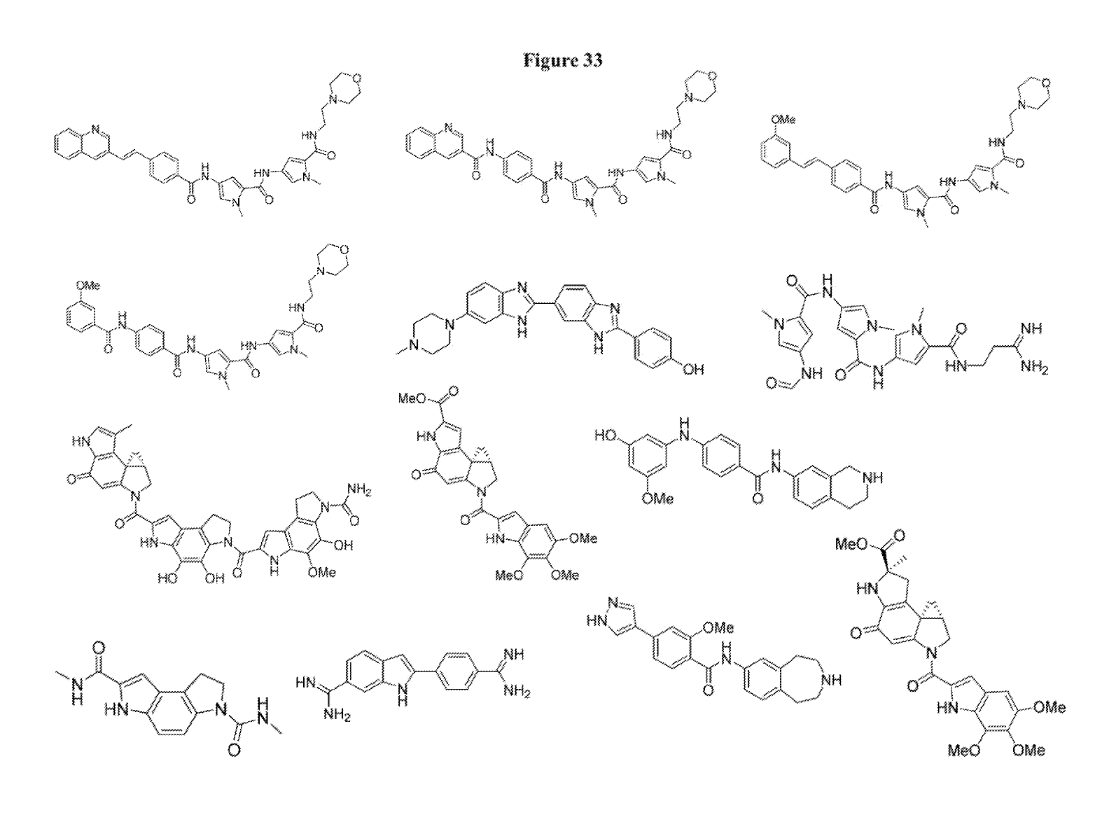

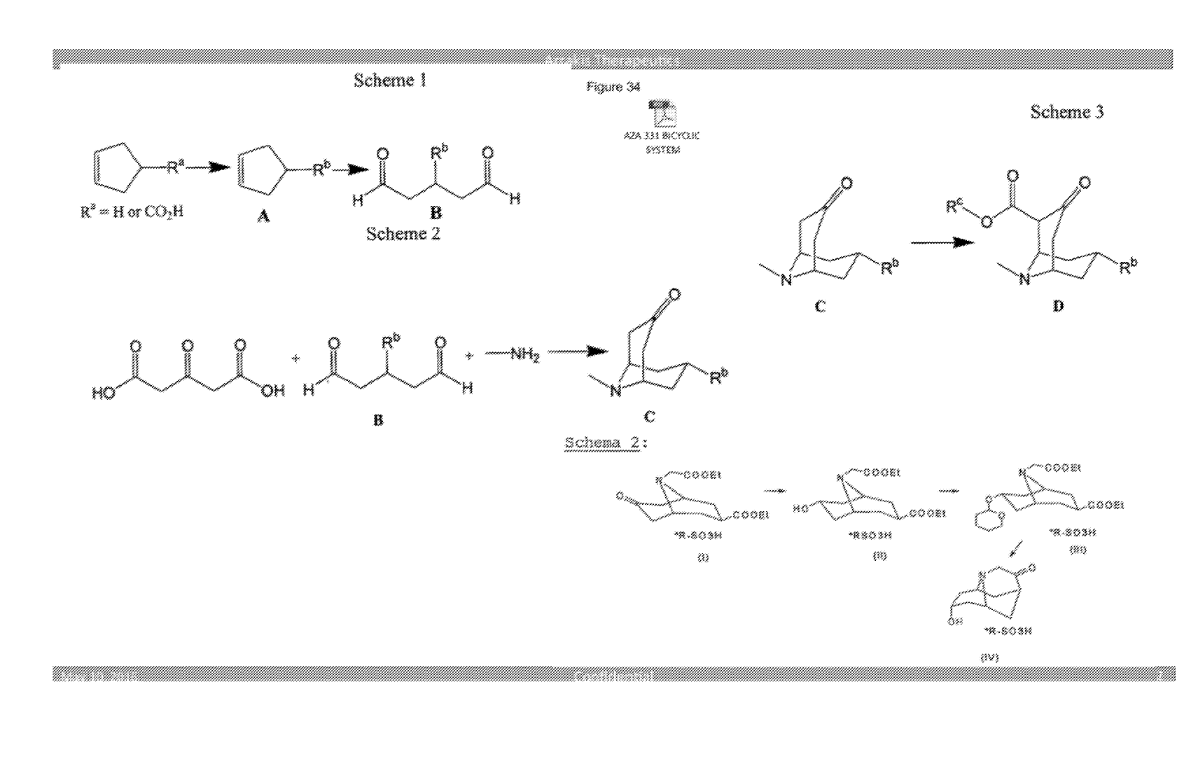

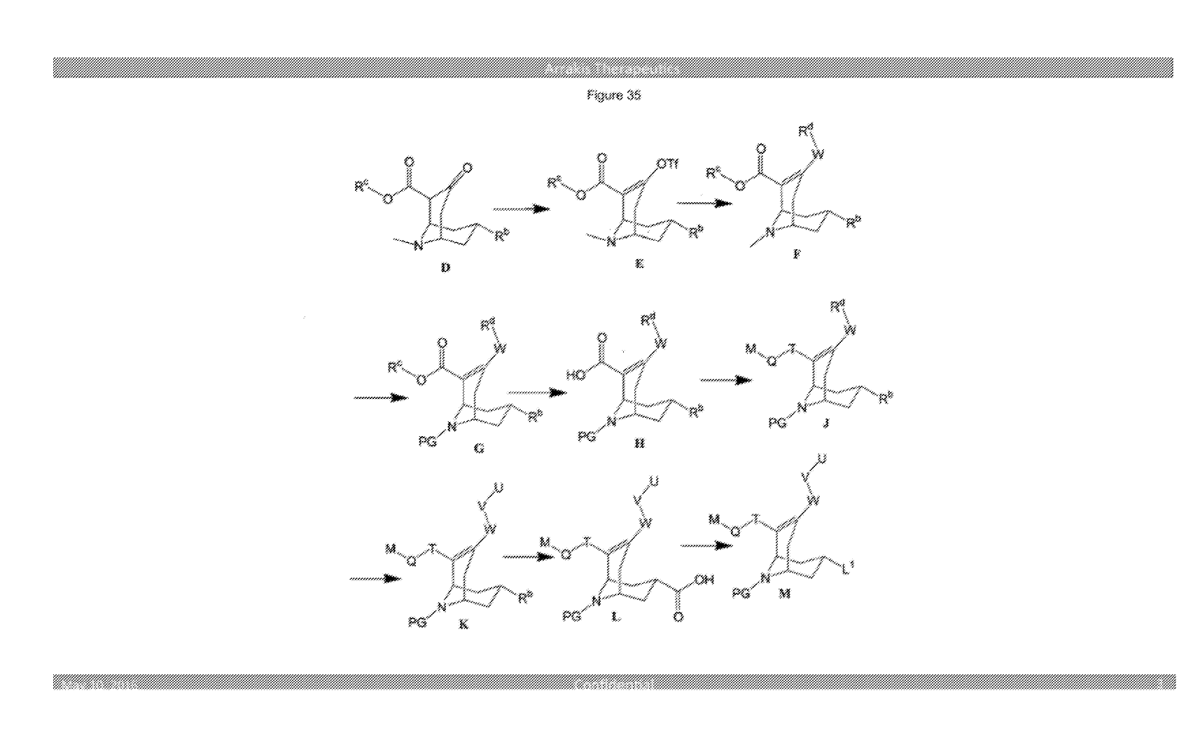

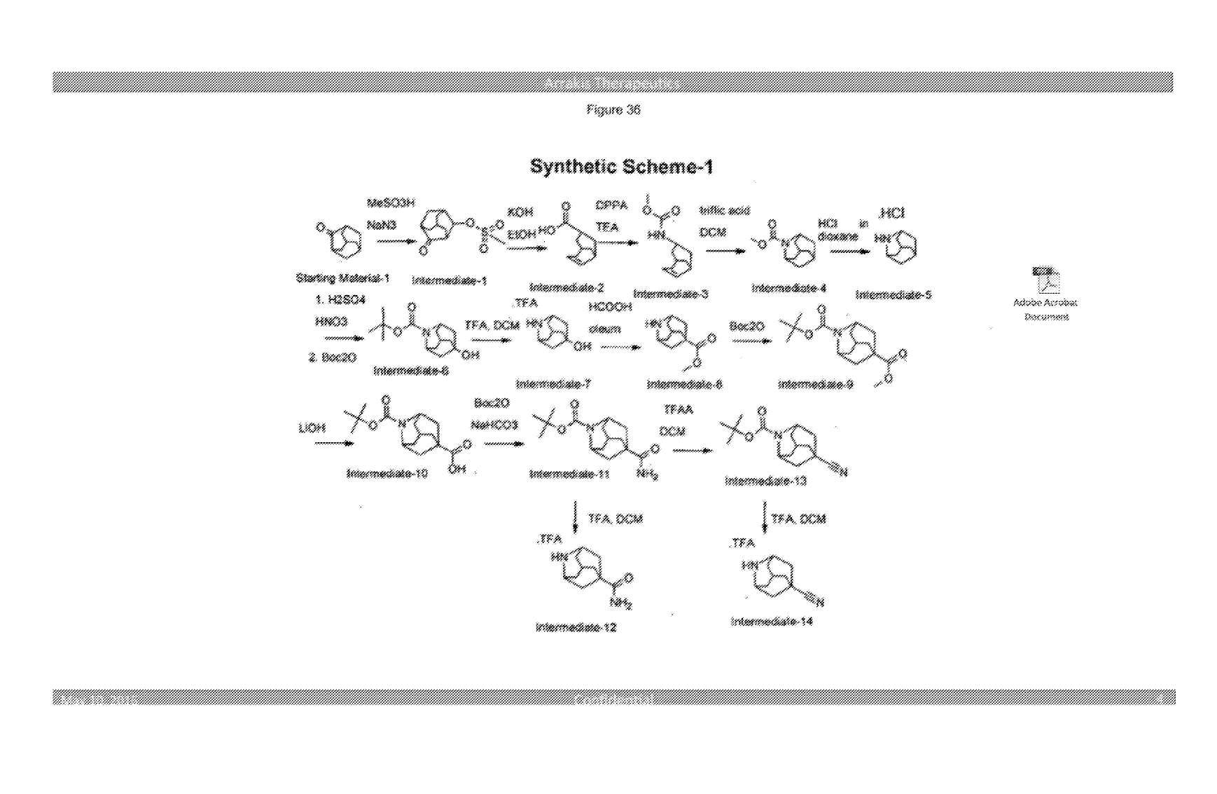

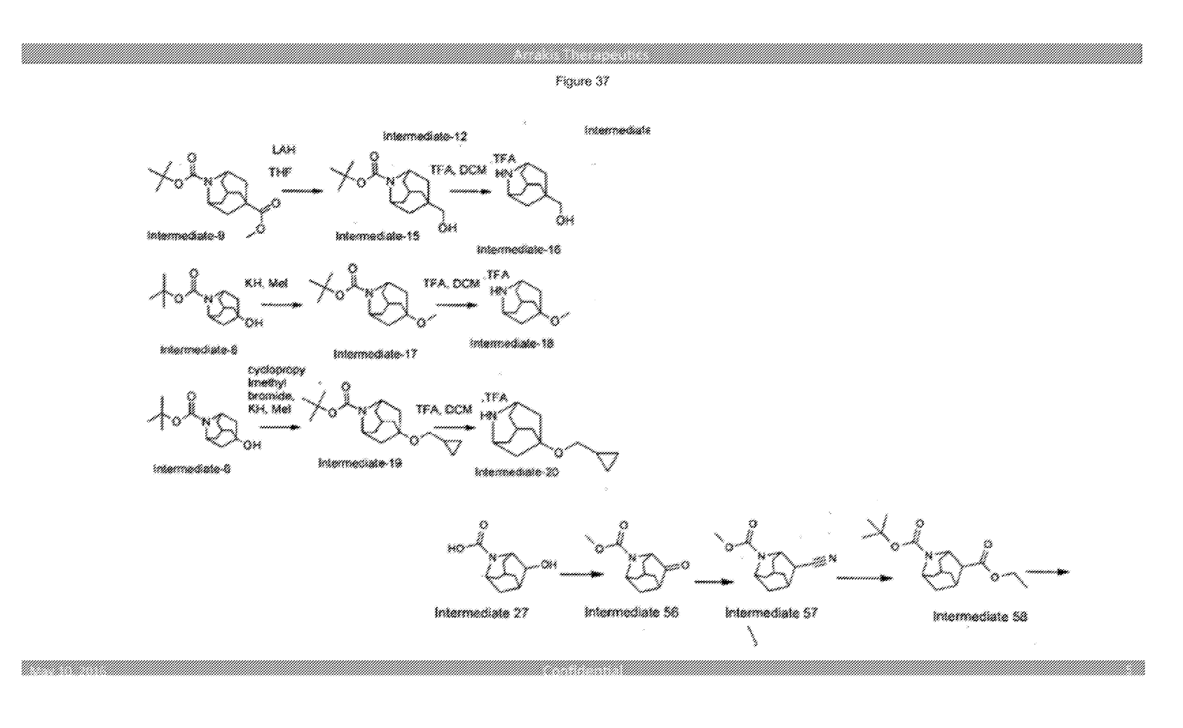

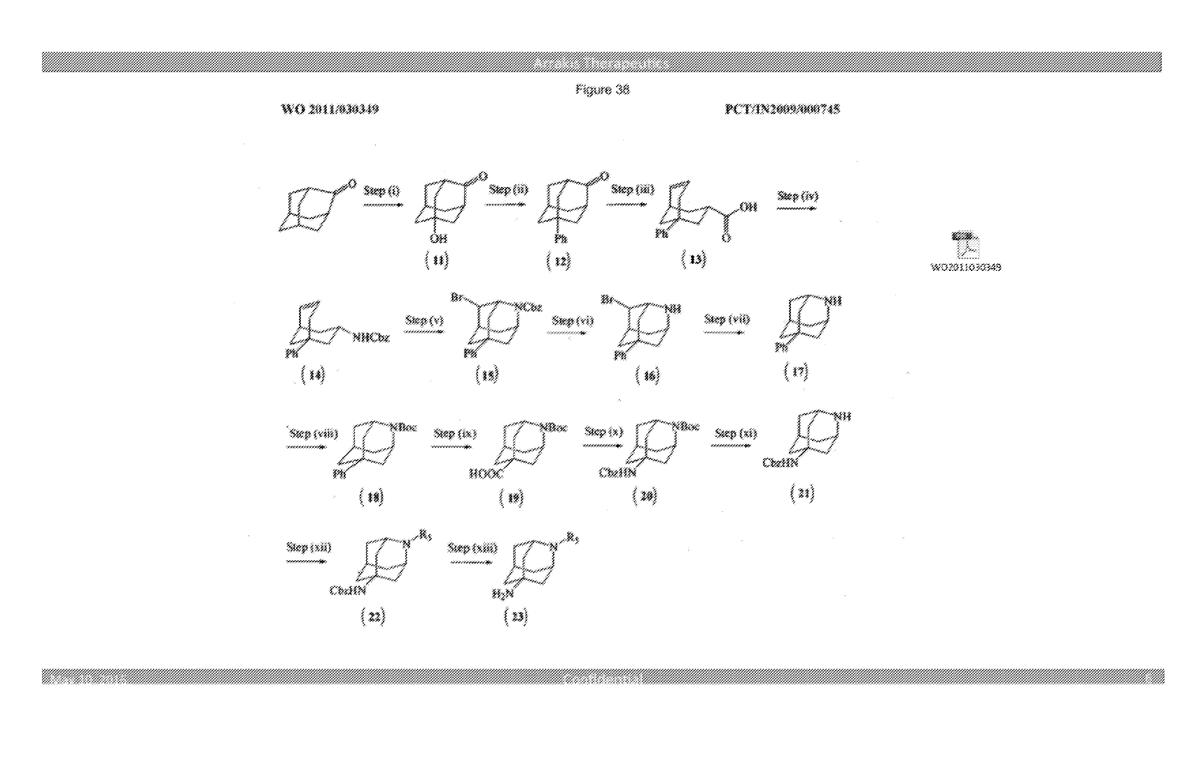

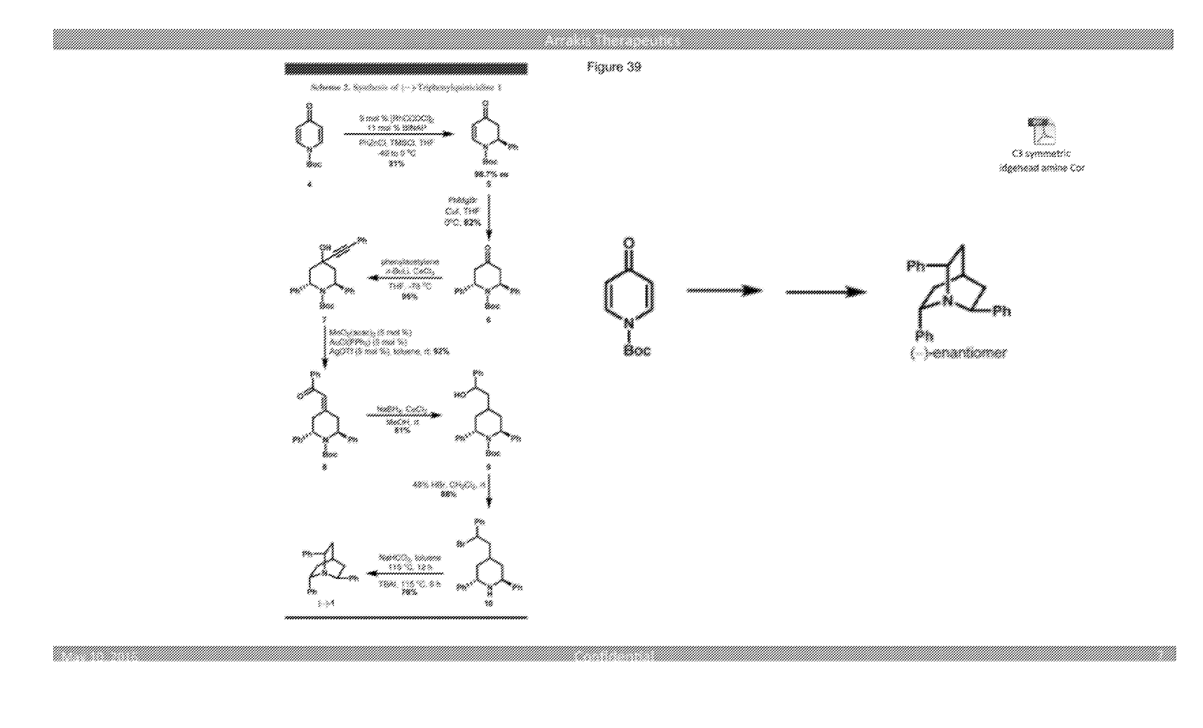

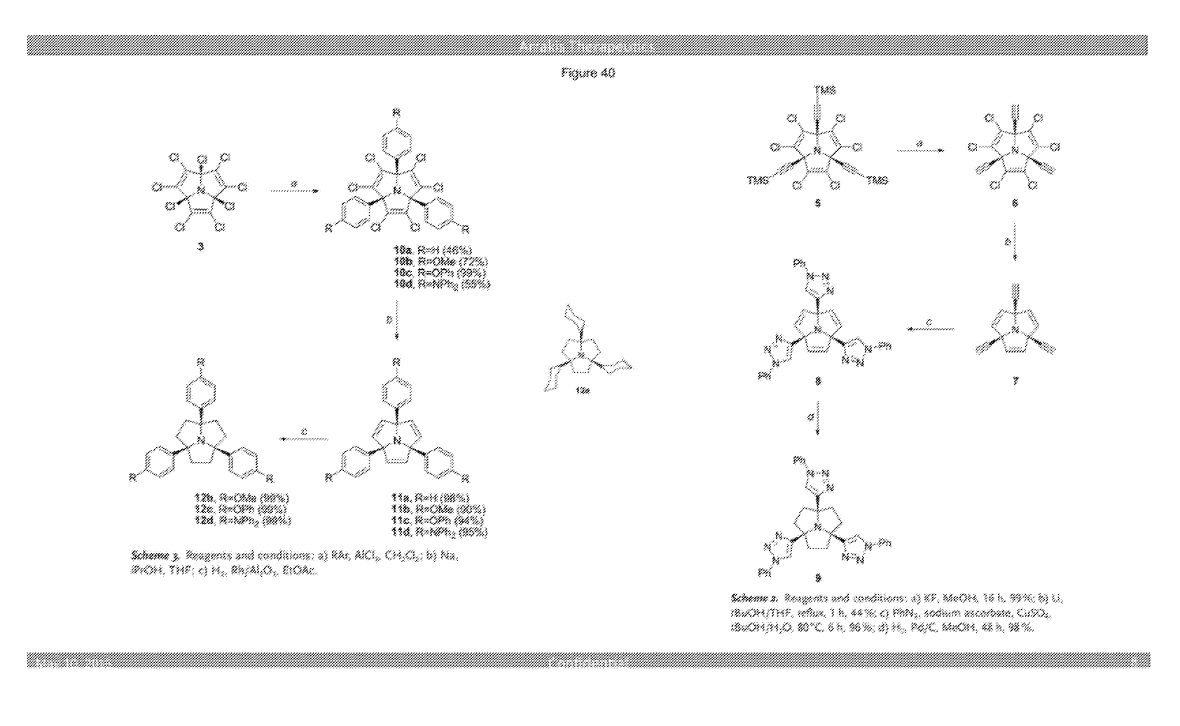

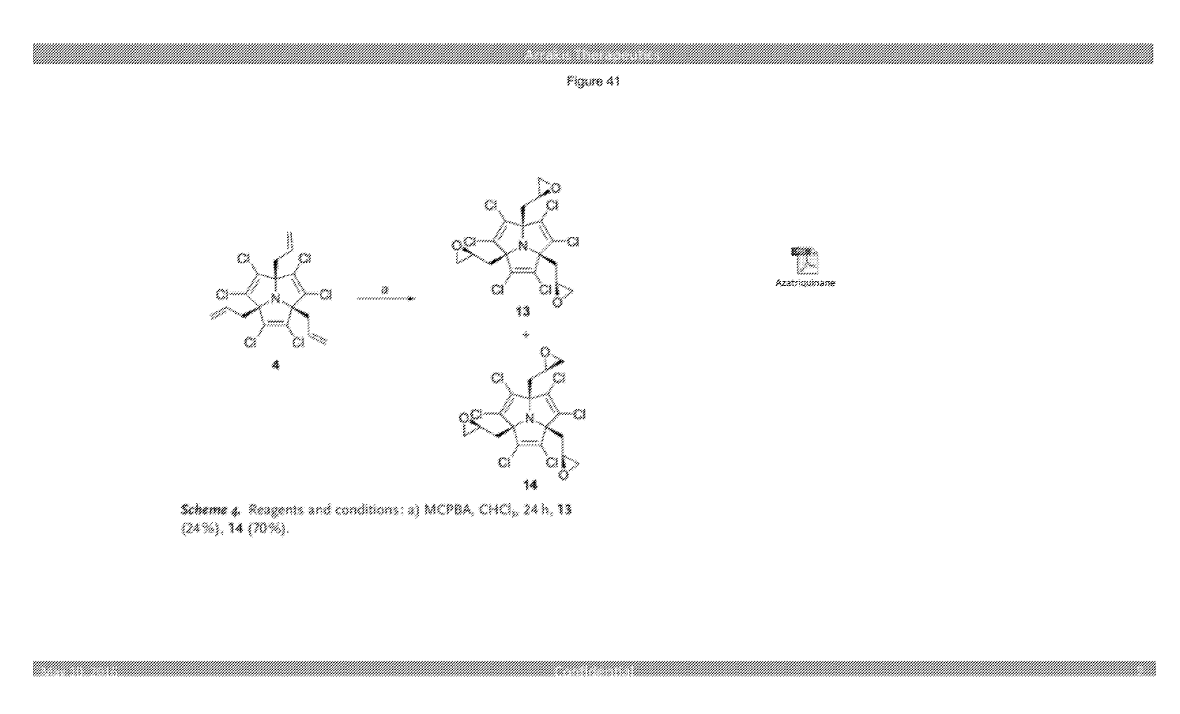

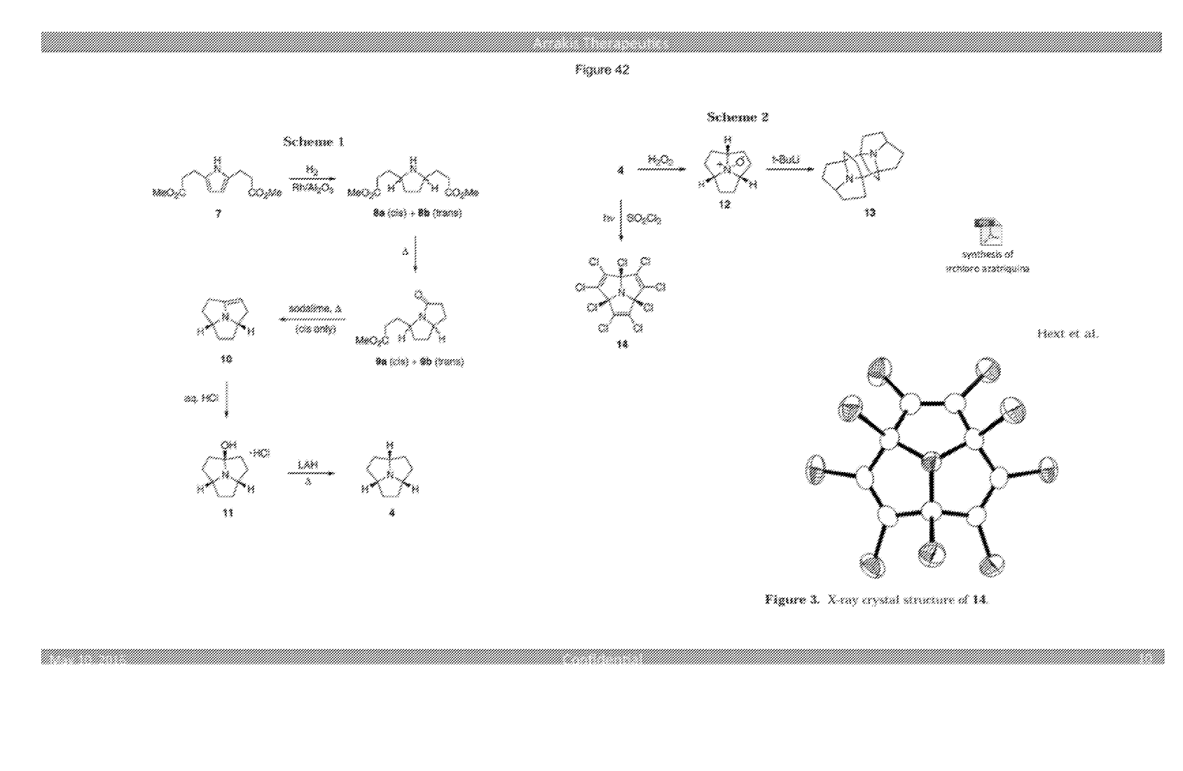

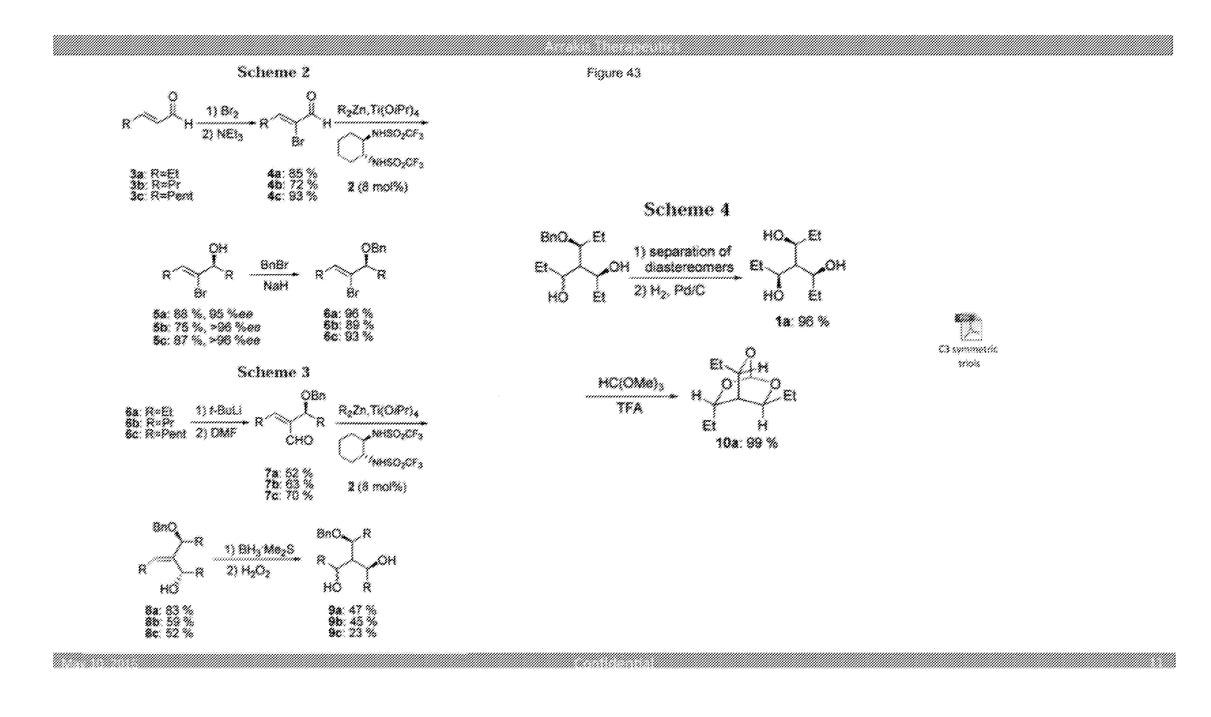

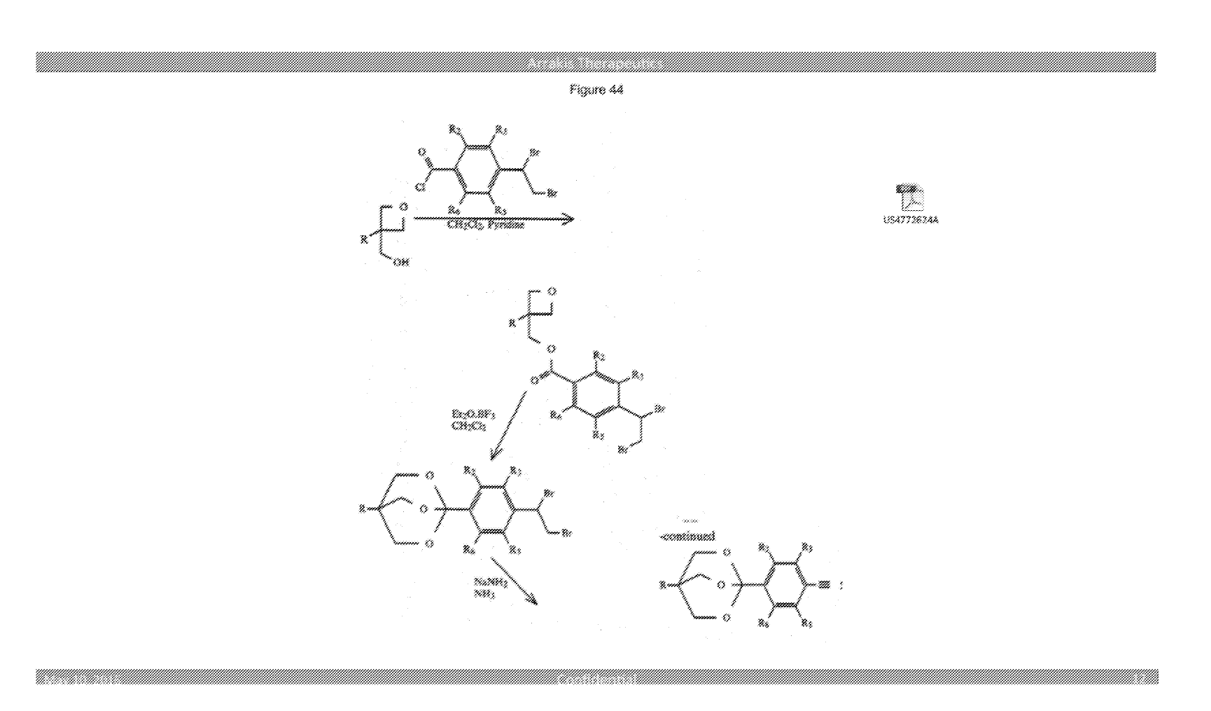

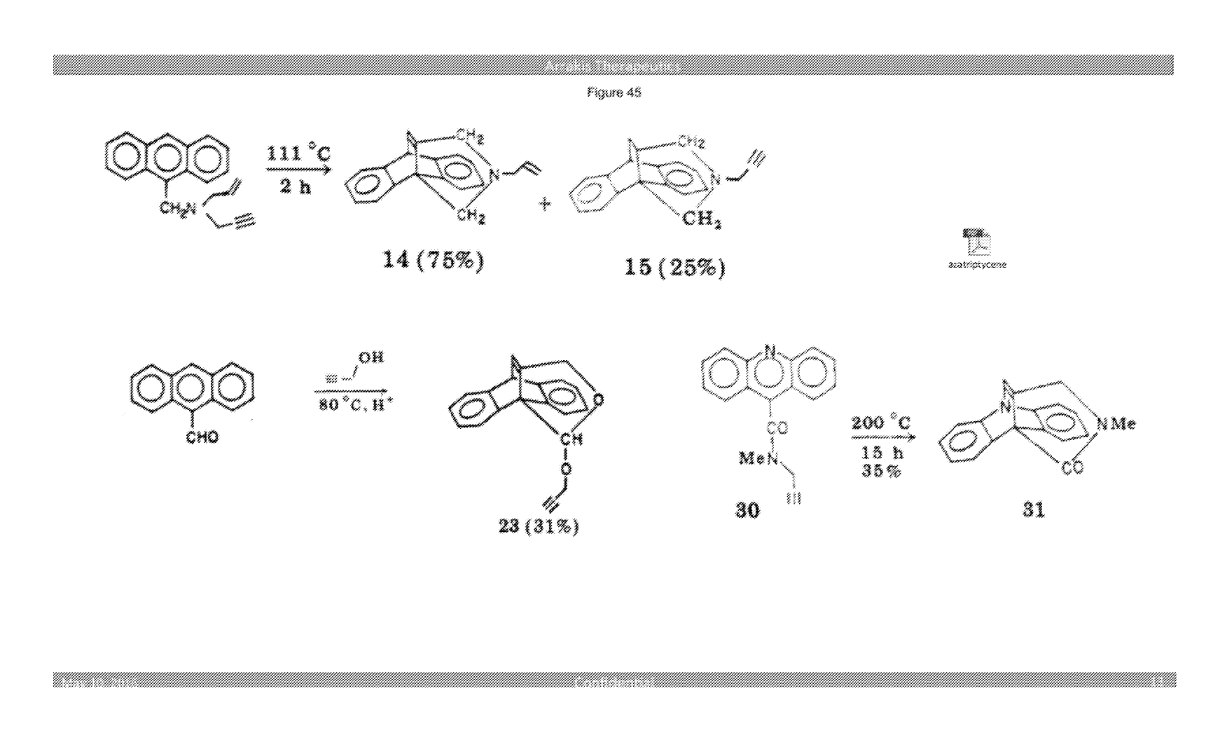

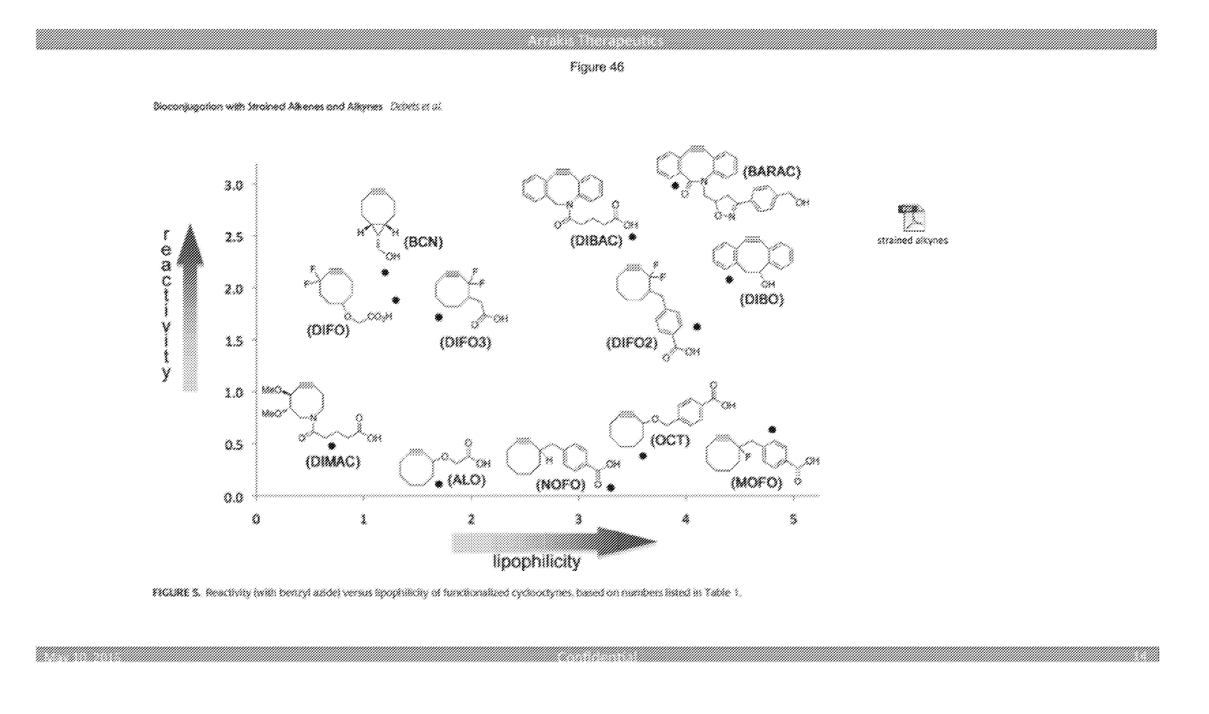

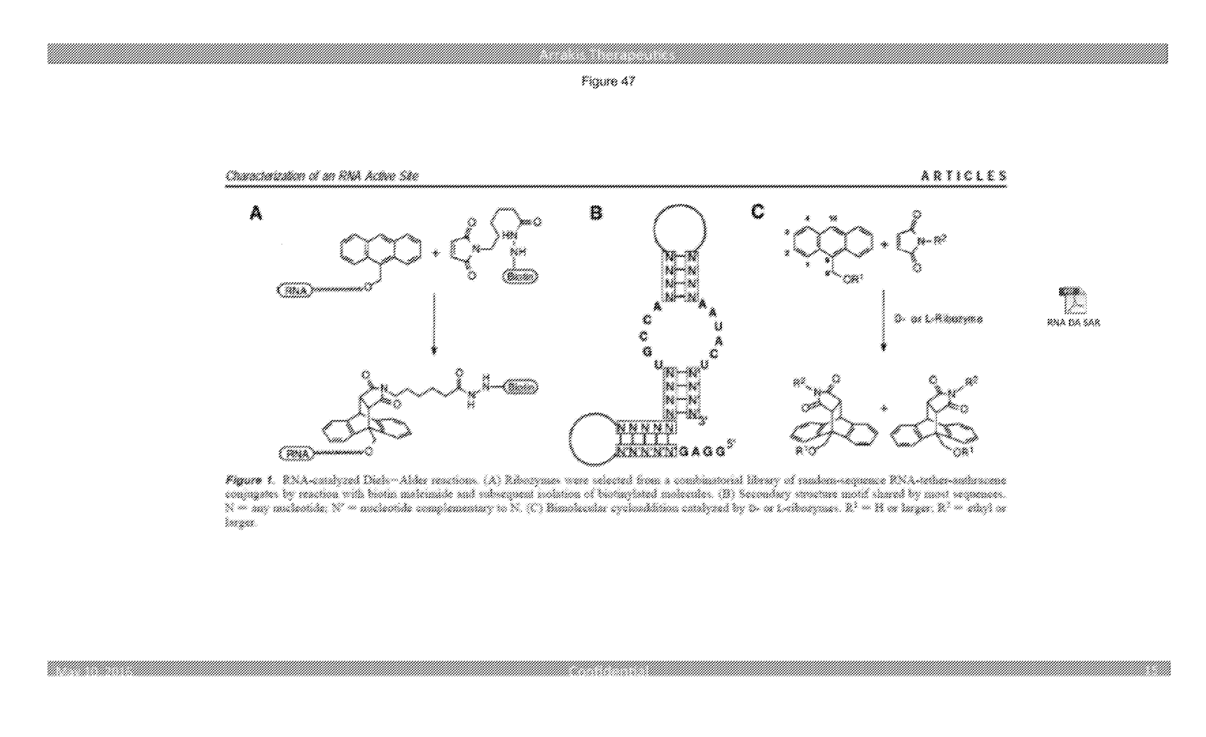

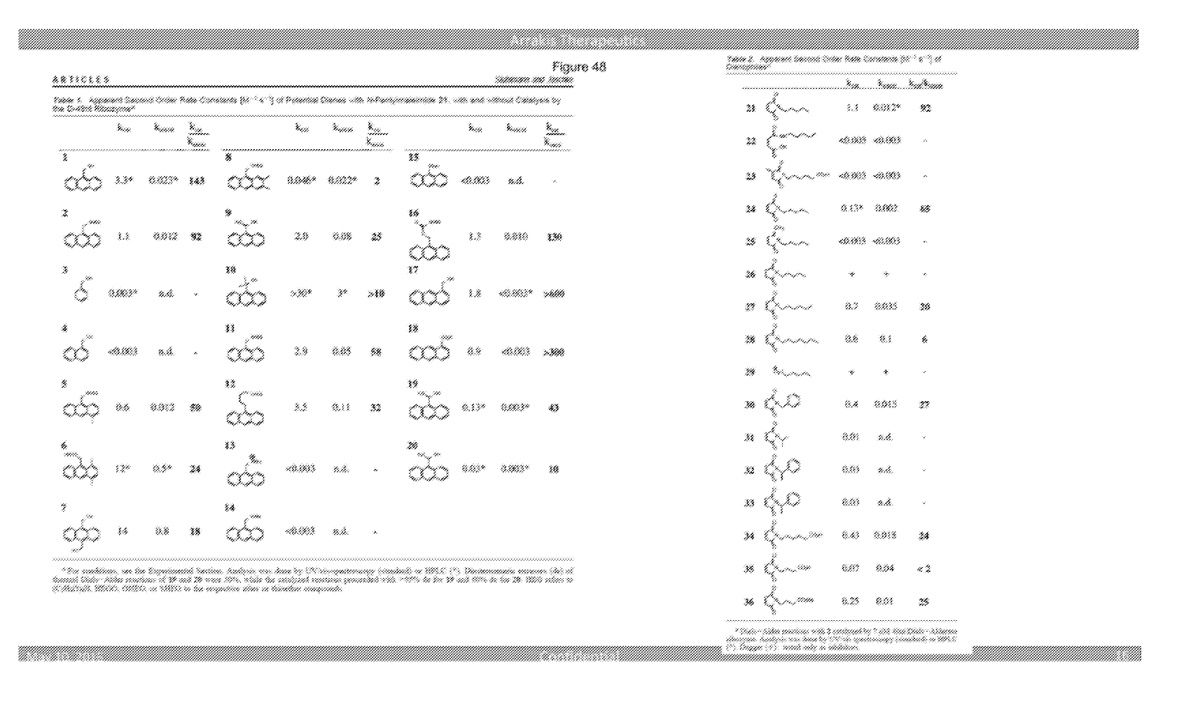

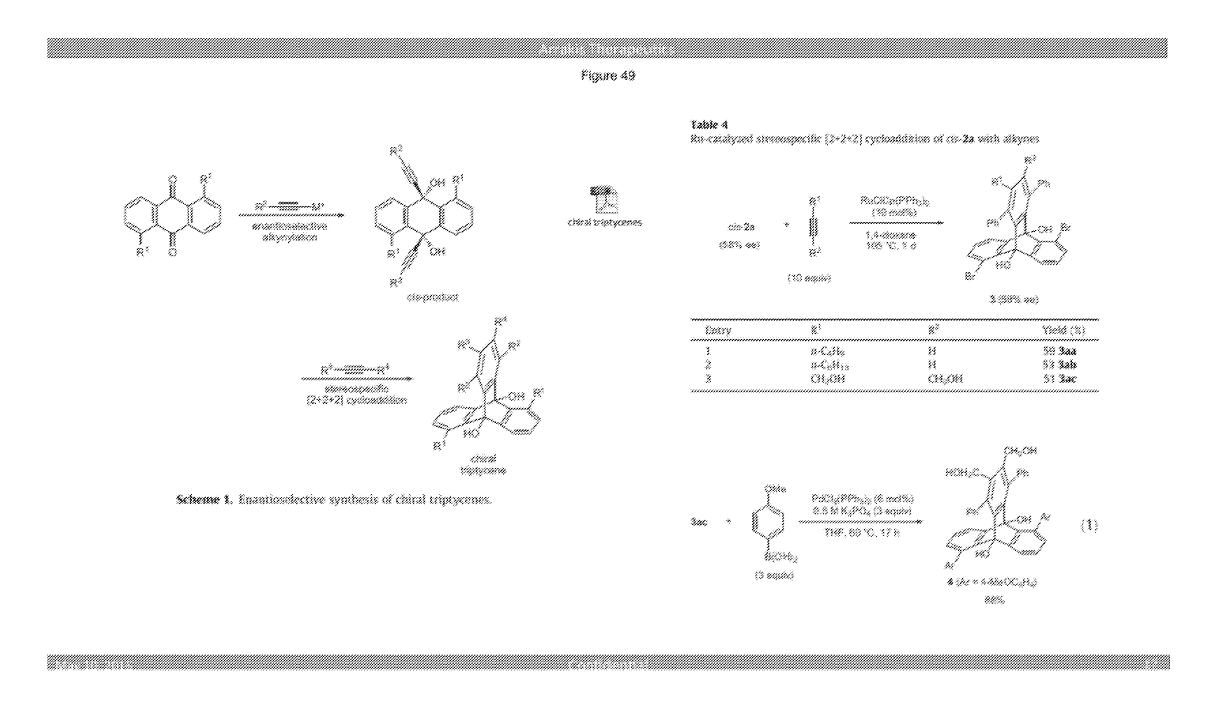

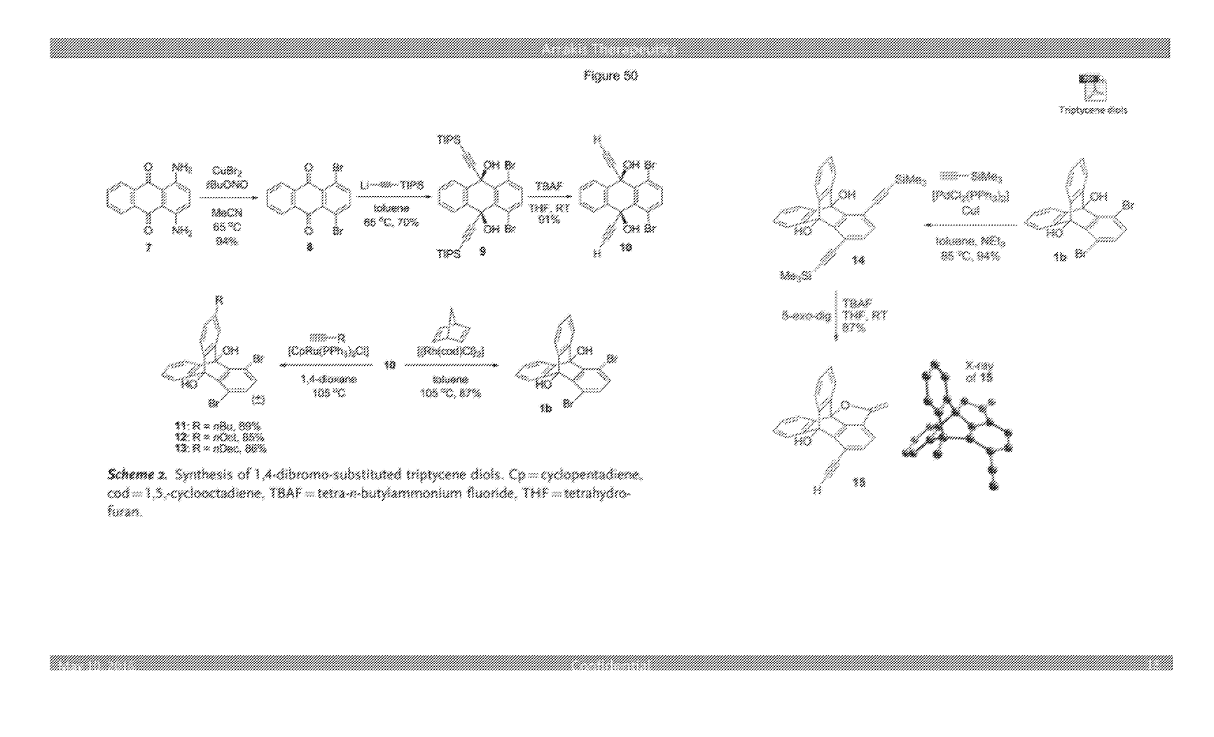

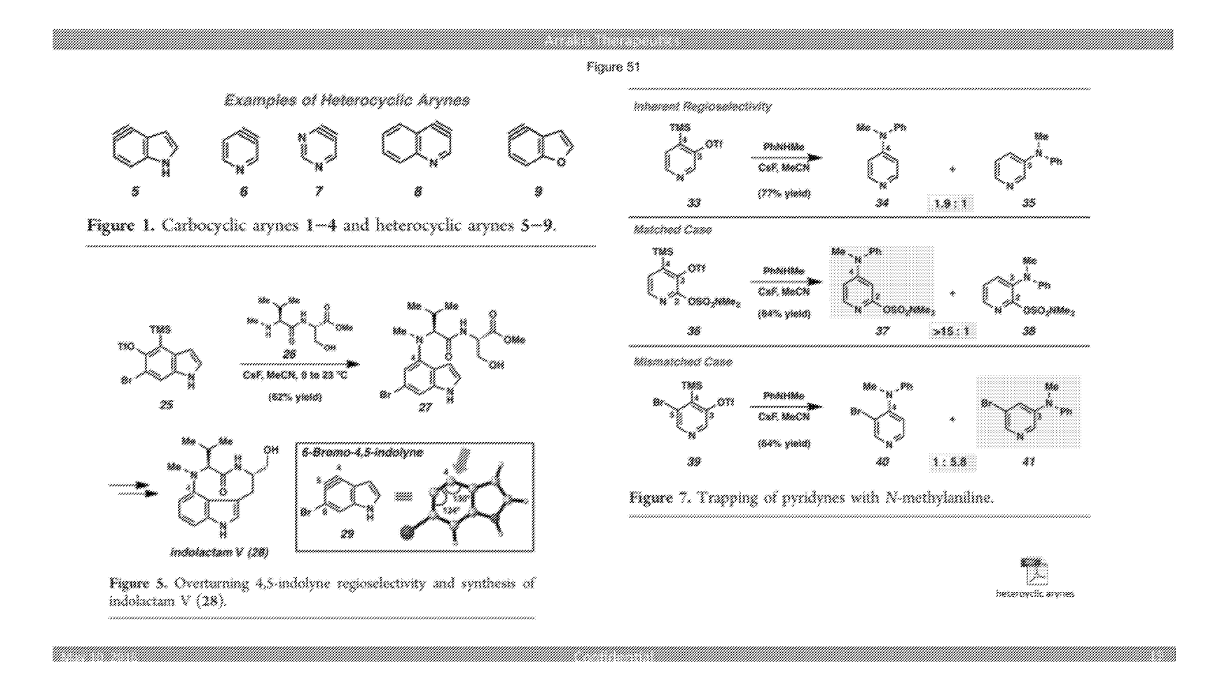

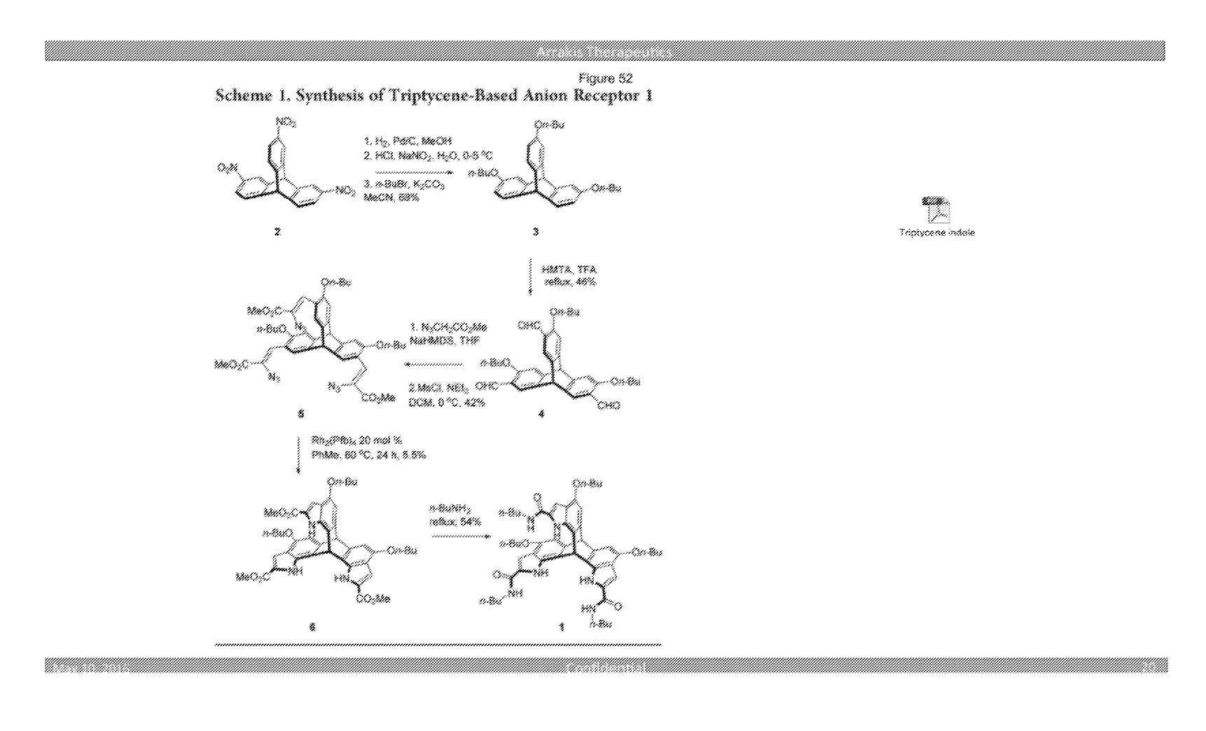

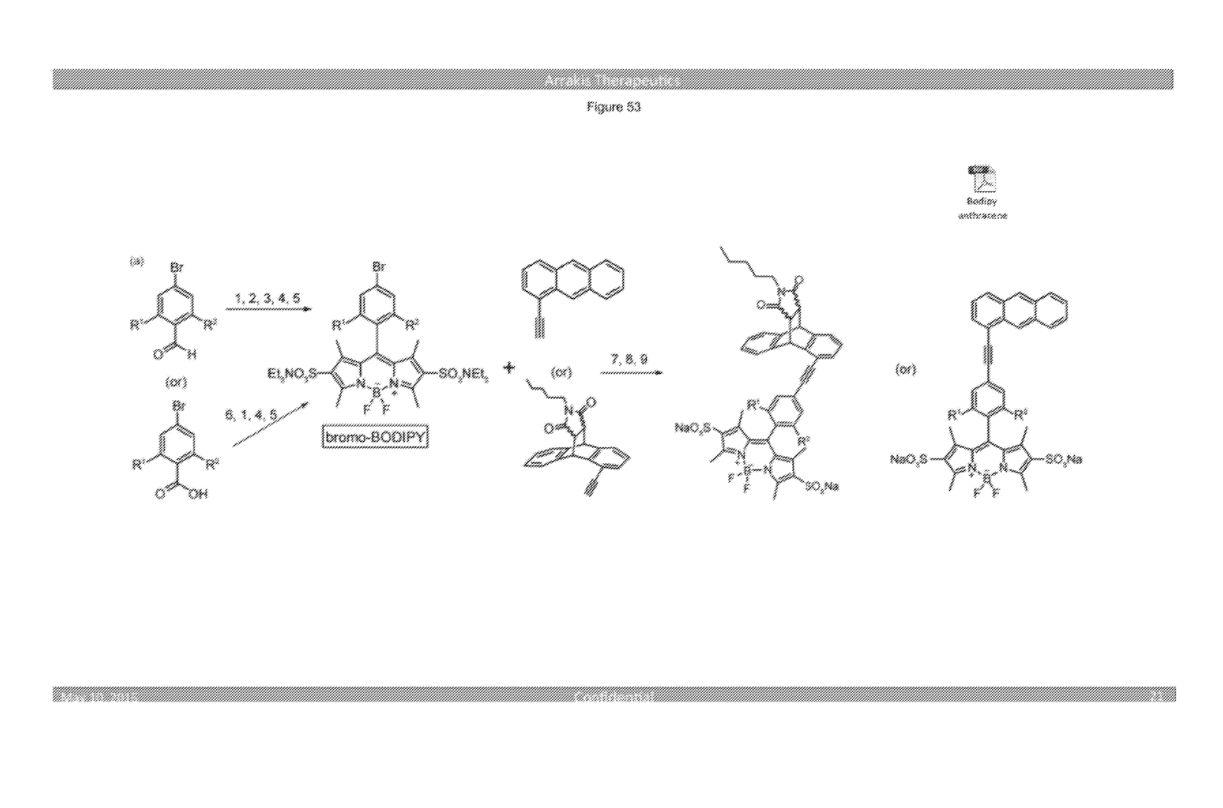

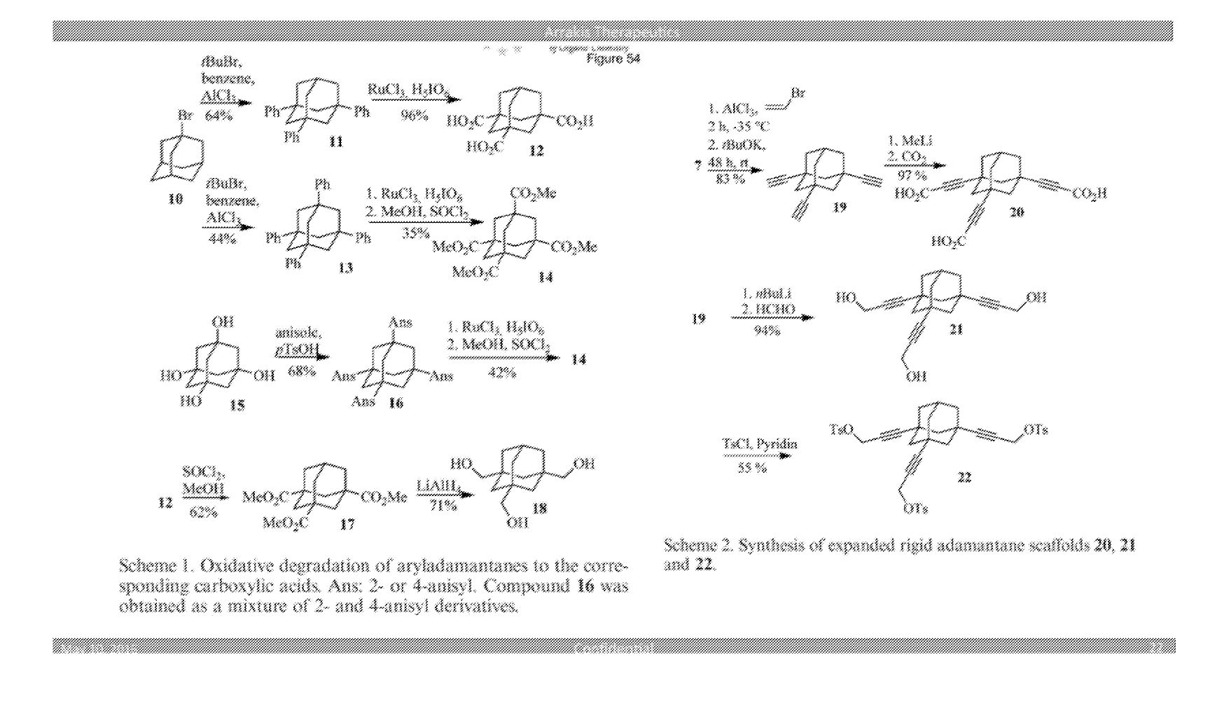

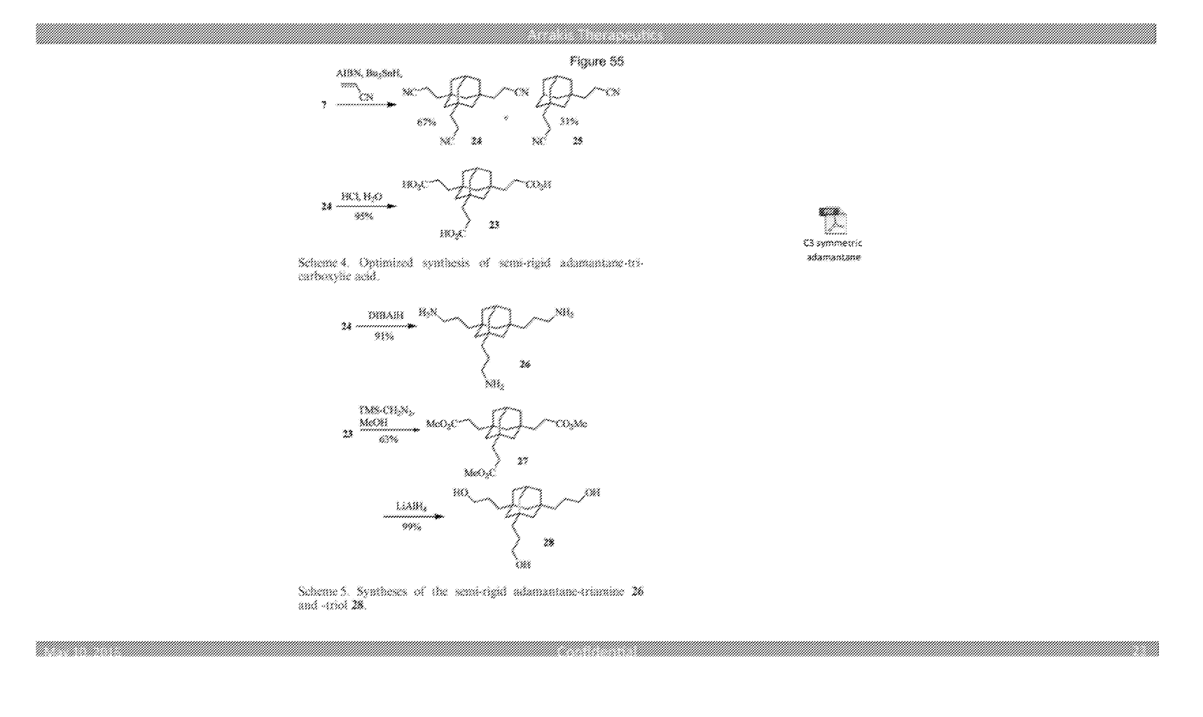

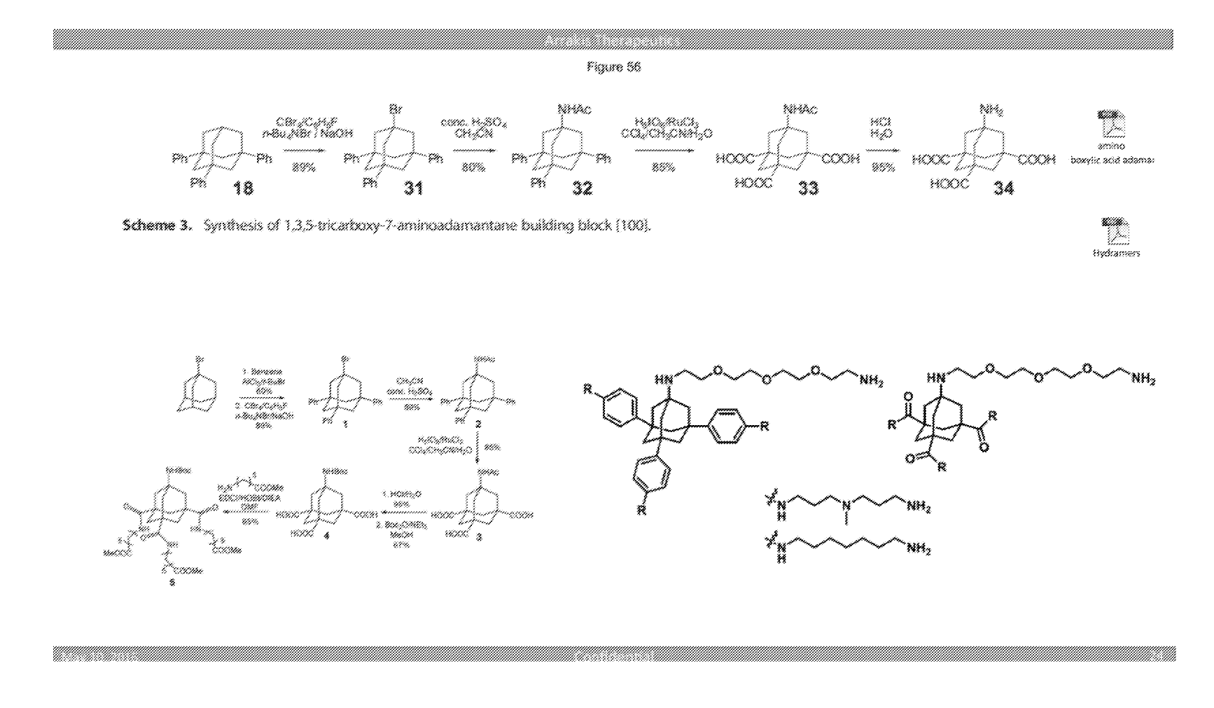

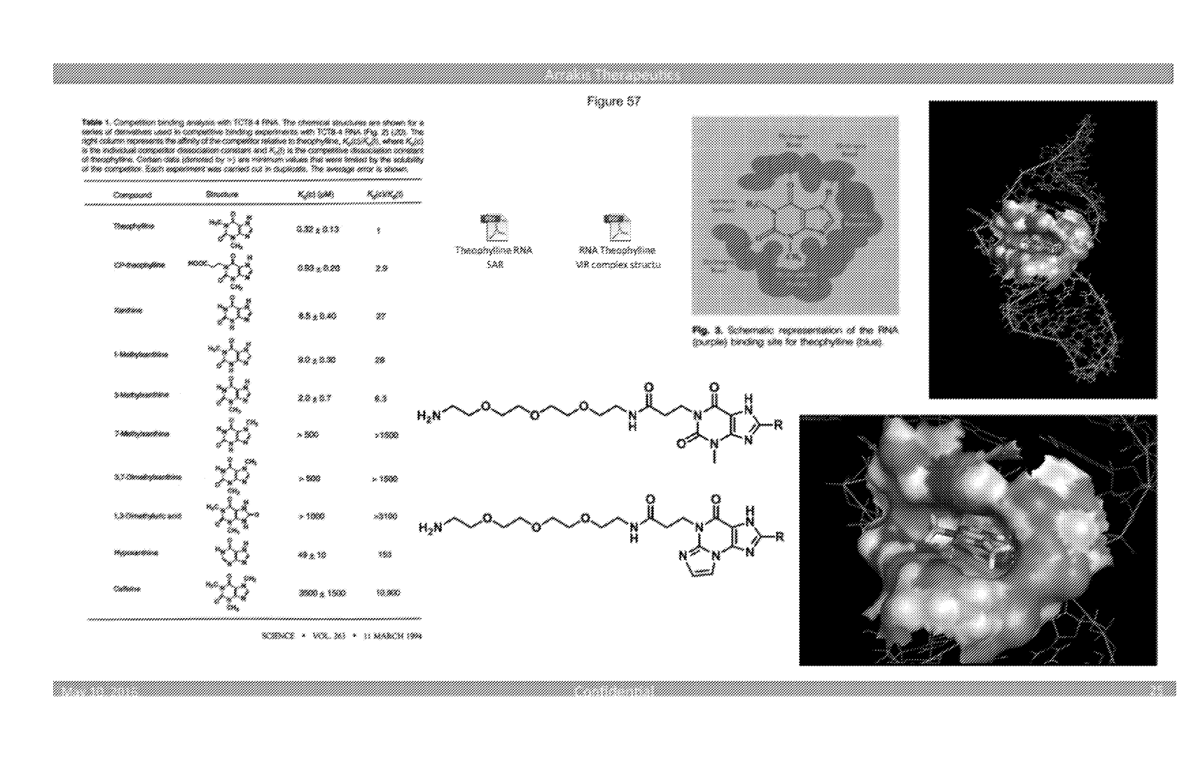

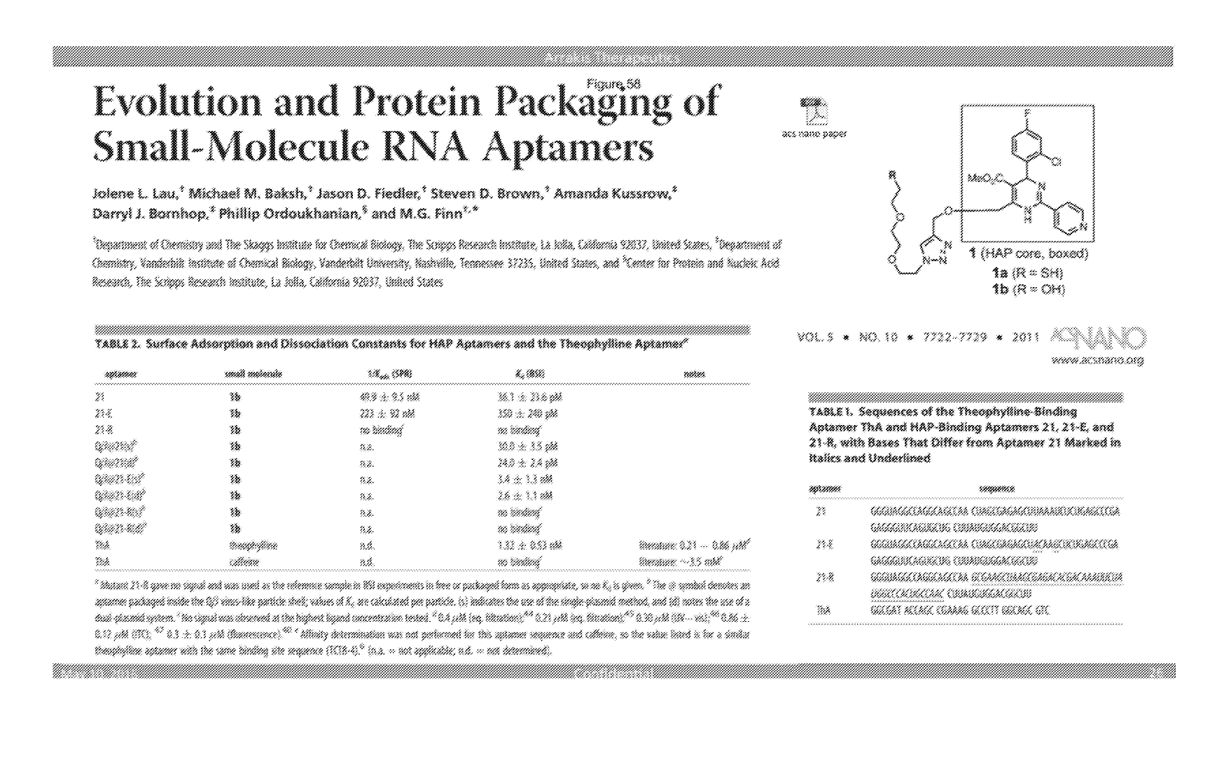

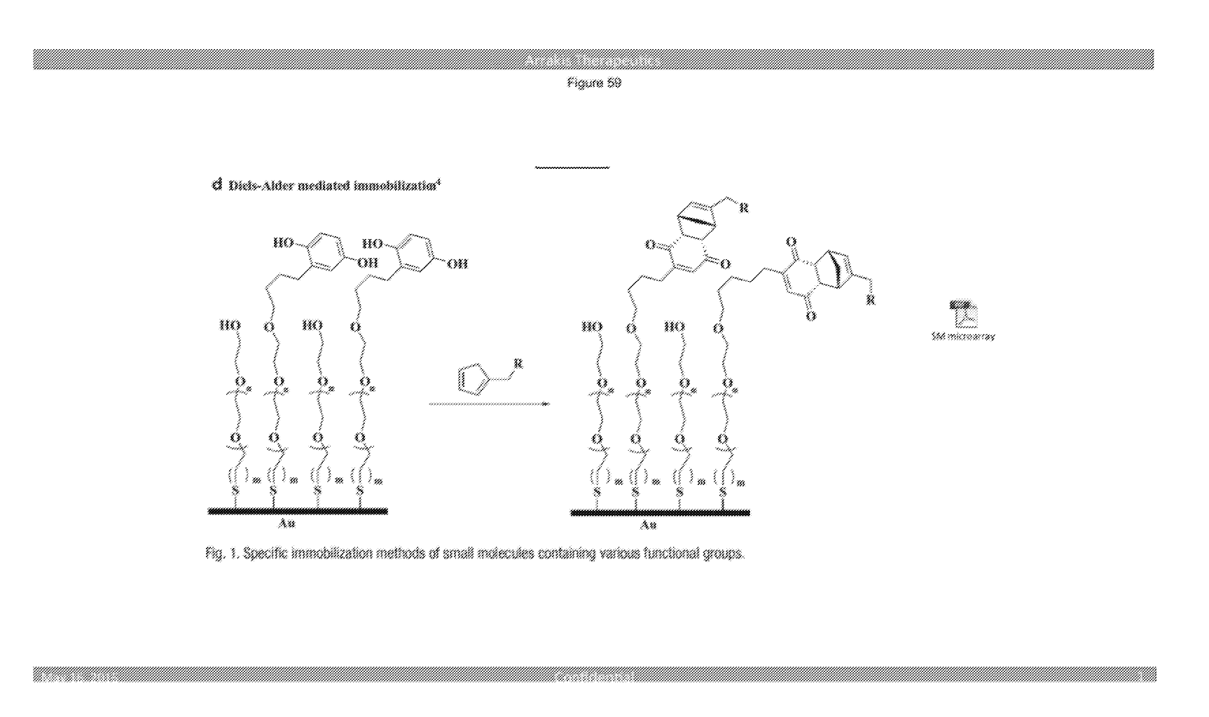

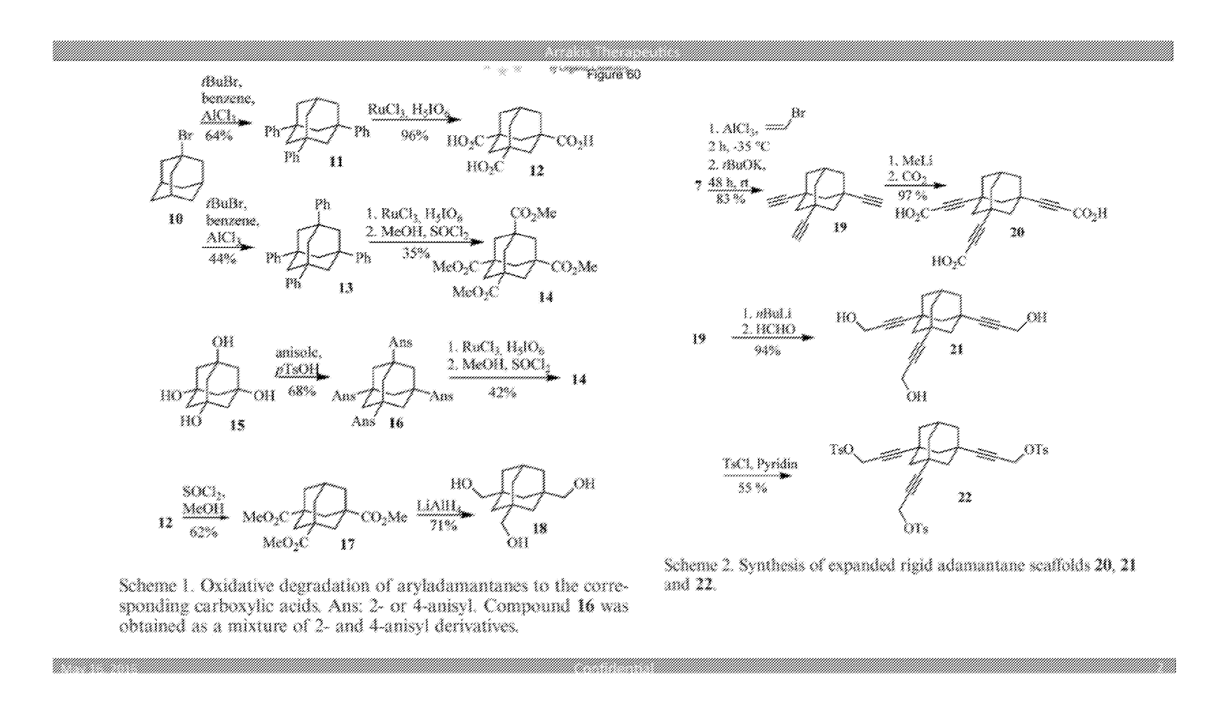

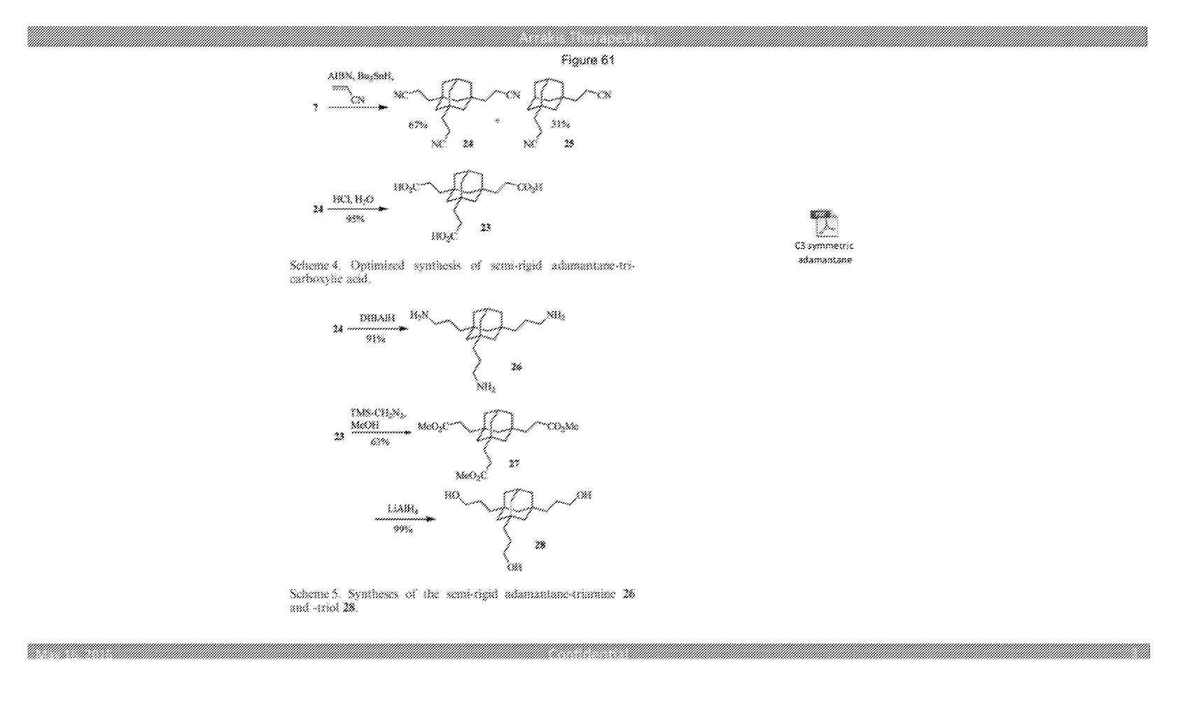

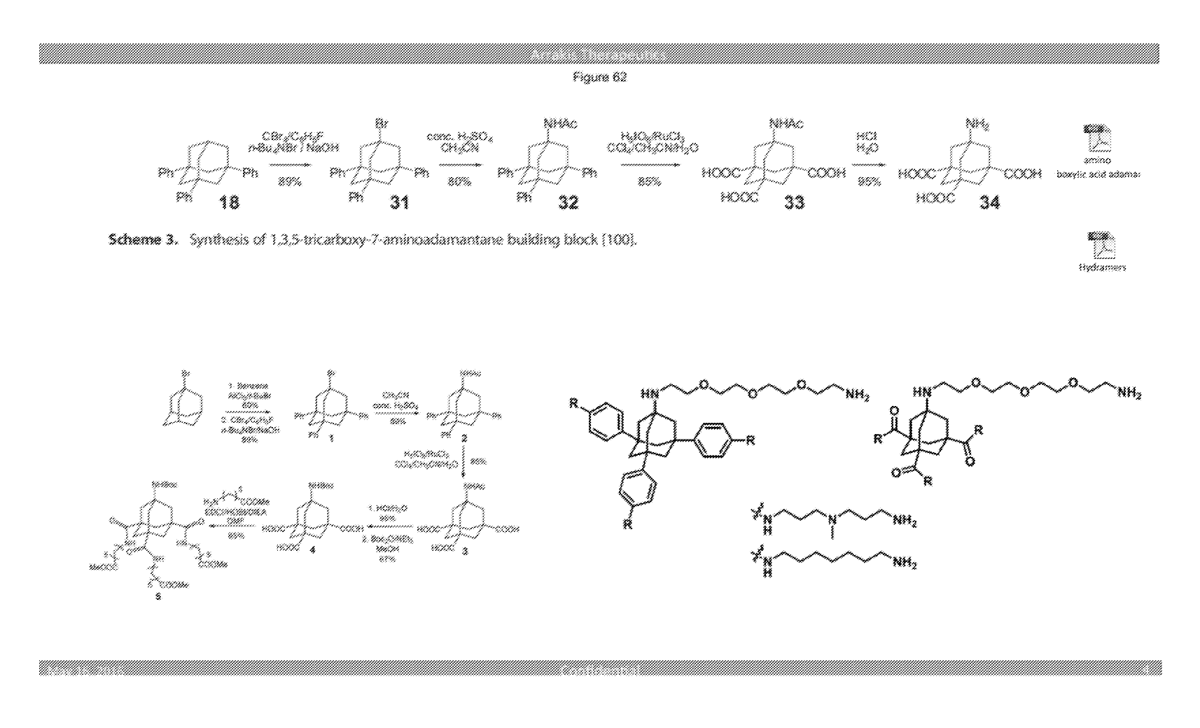

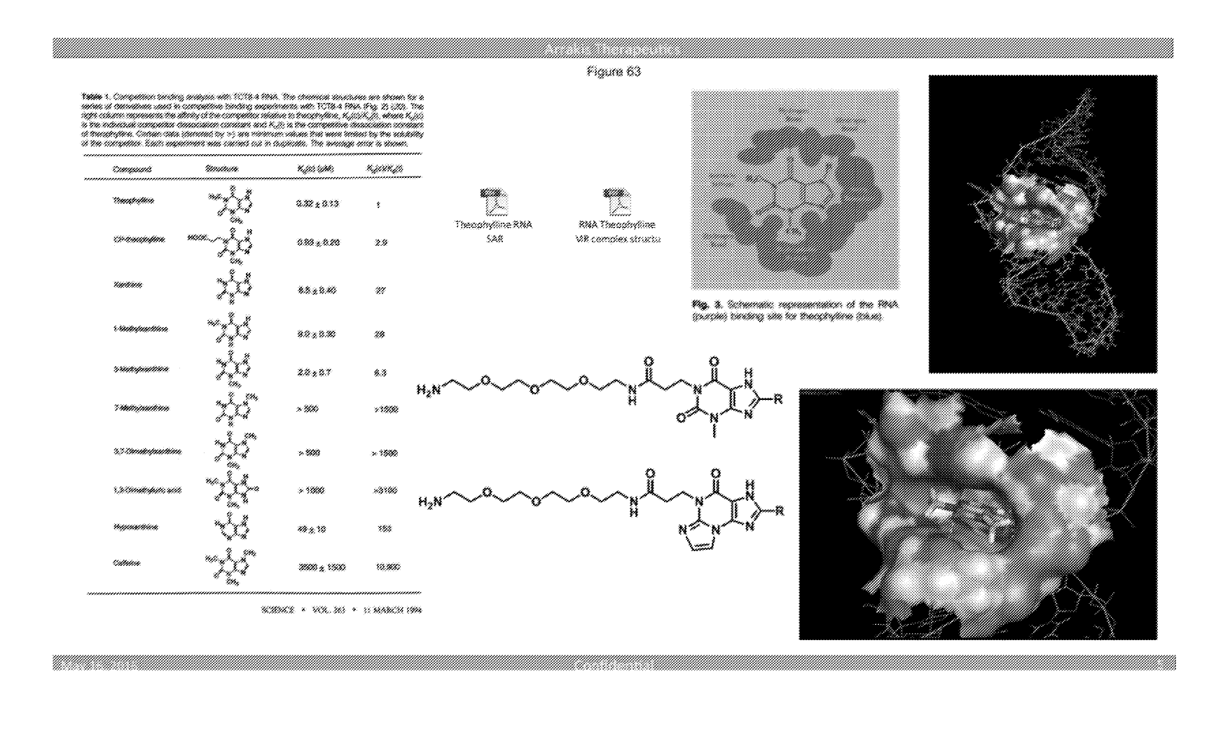

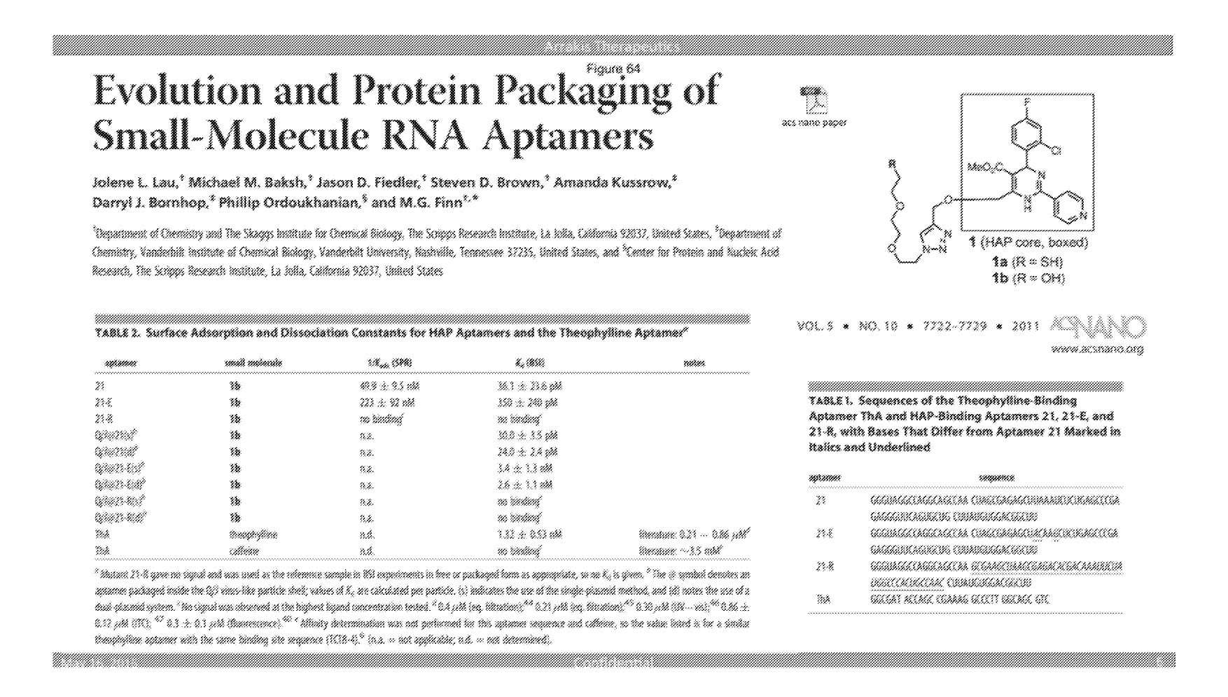

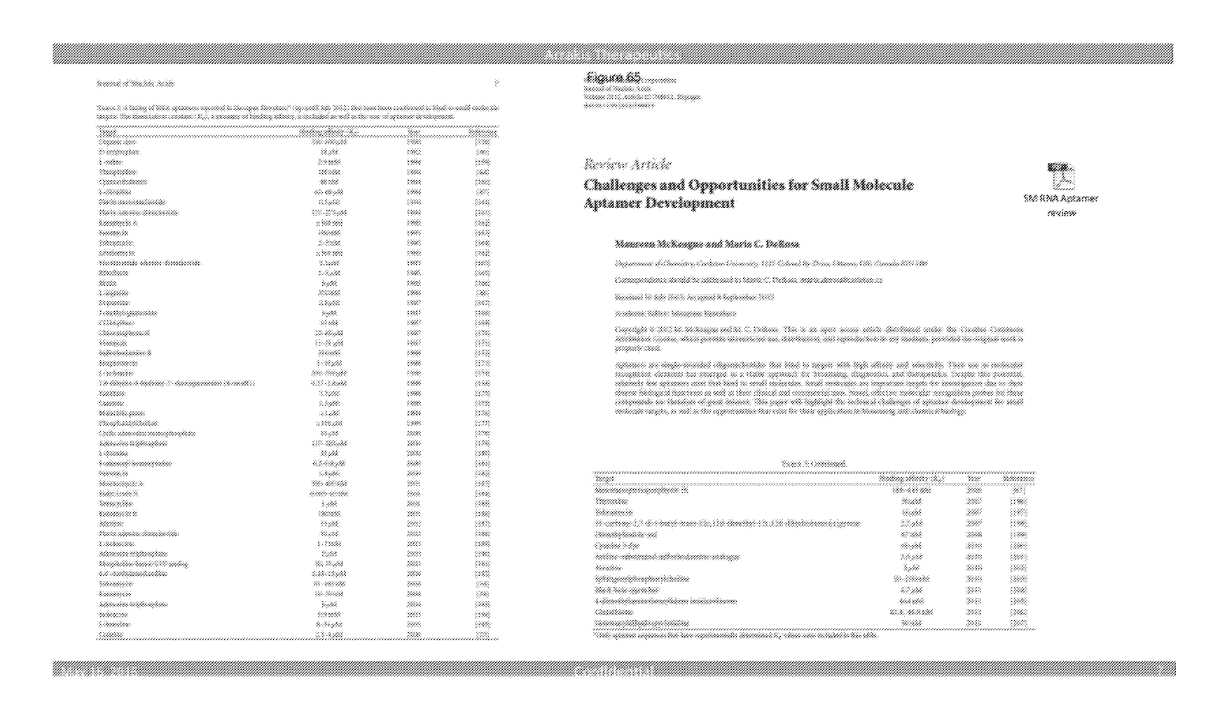

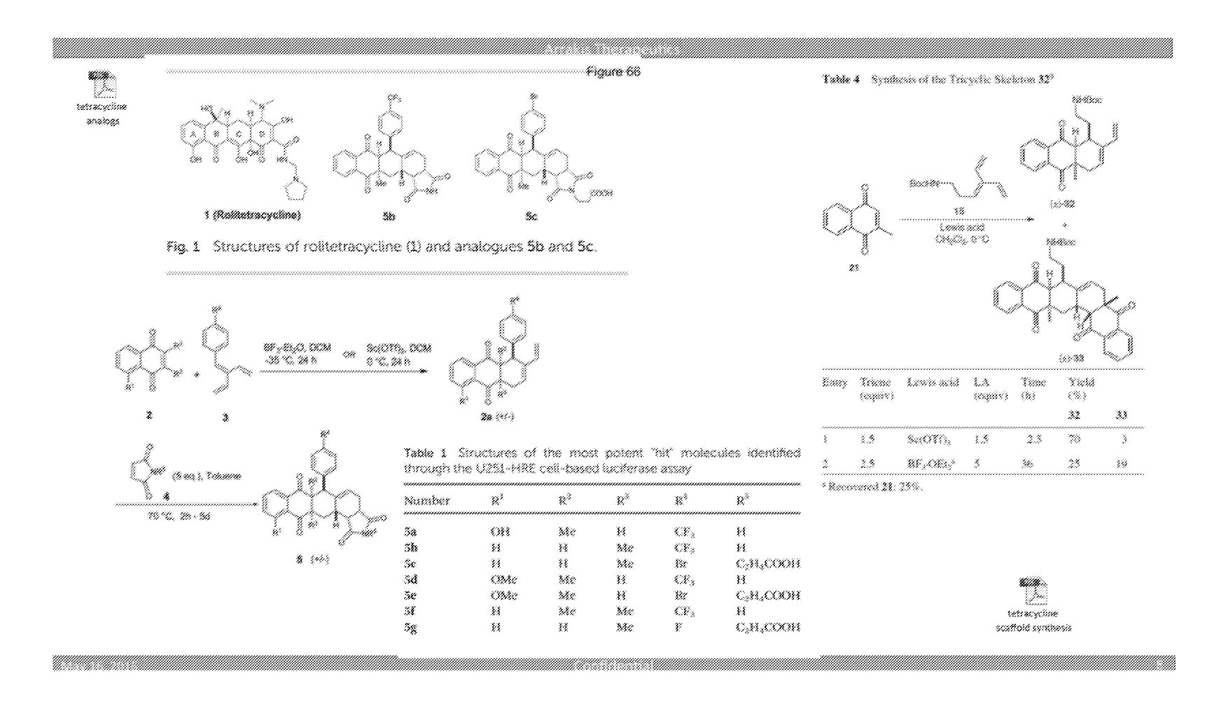

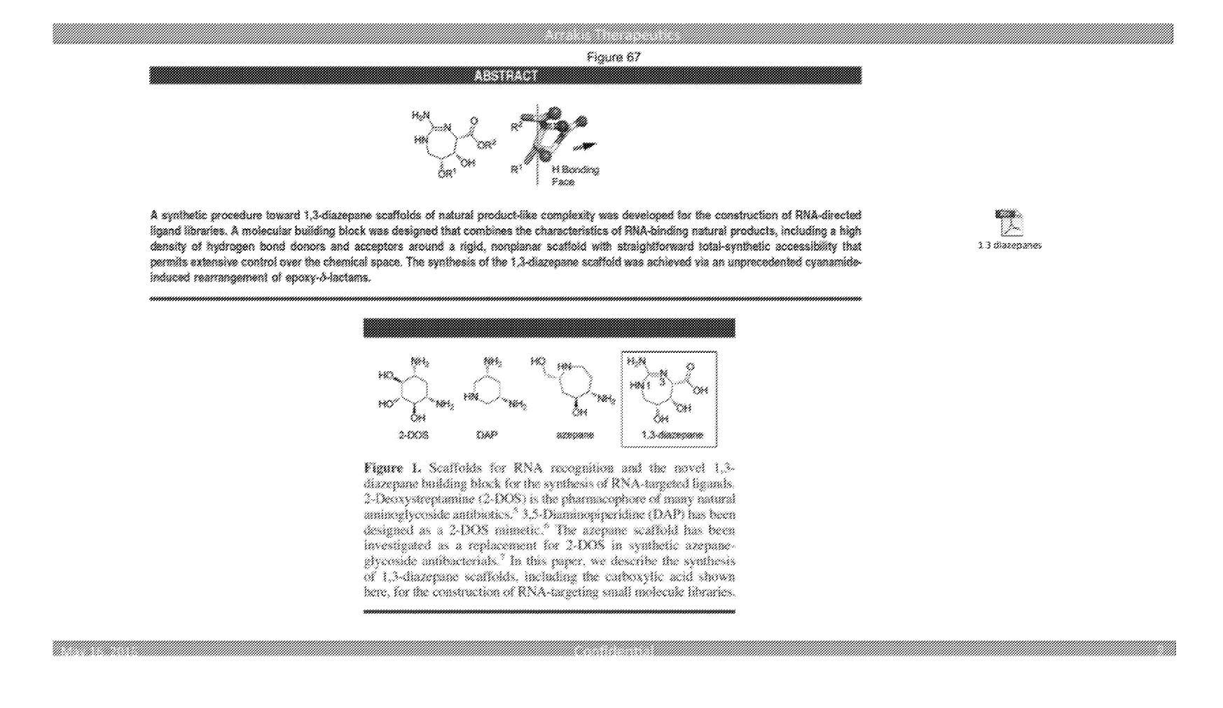

In some embodiments, a compound of Formula I is covalently linked, either directly or through a linker such as L¹ to at least one structure shown in FIG. 24 or 33 by any chemically feasible means.

In another aspect, the present invention provides a compound of Formula II:

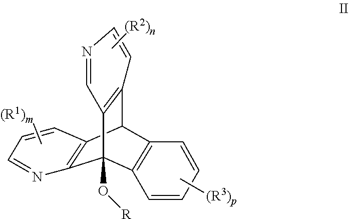

or a pharmaceutically acceptable salt thereof, wherein each of R, R¹, R², R³, R⁶, L¹, L², L³, —Cy-, m, n, and p is as defined above and described in embodiments herein, both singly and in combination.

In some embodiments, a compound of Formula II is covalently linked, either directly or through a linker such as L¹, to at least one structure shown in FIG. 24 or 33 by any chemically feasible means.

In some embodiments, the present invention provides a compound of Formula III:

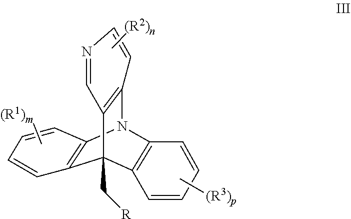

or a pharmaceutically acceptable salt thereof, wherein each of R, R¹, R², R³, R⁶, L¹, L², L³, —Cy-, m, n, and p is as defined above and described in embodiments herein, both singly and in combination.

In some embodiments, a compound of Formula III is covalently linked, either directly or through a linker such as L¹, to at least one structure shown in FIG. 24 or 33 by any chemically feasible means.

In some embodiments, the present invention provides a compound of Formula IV:

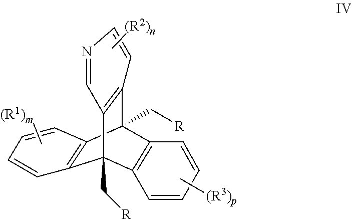

or a pharmaceutically acceptable salt thereof, wherein each of R, R¹, R², R³, R⁶, L¹, L², L³, —Cy-, m, n, and p is as defined above and described in embodiments herein, both singly and in combination.

In some embodiments, a compound of Formula IV is covalently linked, either directly or through a linker such as L¹, to at least one structure shown in FIG. 24 or 33 by any chemically feasible means.

In some embodiments, the present invention provides a compound of Formula V.

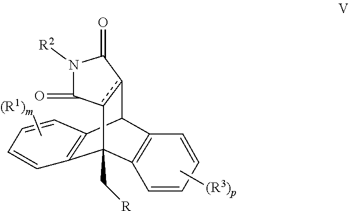

or a pharmaceutically acceptable salt thereof, wherein each of R, R¹, R², R³, R⁶, L¹, L², L³, —Cy-, m, and p is as defined above and described in embodiments herein, both singly and in combination.

In some embodiments, a compound of Formula V is covalently linked, either directly or through a linker such as L¹, to at least one structure shown in FIG. 24 or 33 by any chemically feasible means.

In some embodiments, the present invention provides a compound of Formula VI:

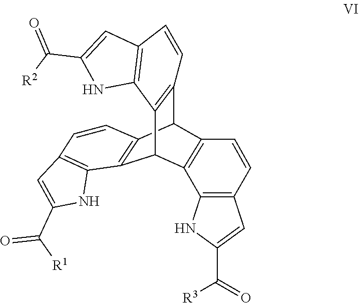

or a pharmaceutically acceptable salt thereof, wherein each of R, R¹, R², R³, R⁶, L¹, L², L³, and —Cy- is as defined above and described in embodiments herein, both singly and in combination.

In some embodiments, a compound of Formula VI is covalently linked, either directly or through a linker such as L¹, to at least one structure shown in FIG. 24 or 33 by any chemically feasible means.

In some embodiments, the present invention provides a compound of Formula V:

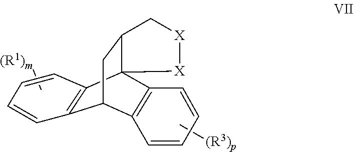

or a pharmaceutically acceptable salt thereof, wherein each of R, R¹, R³, R⁶, L¹, L³, —Cy-, m, and p is as defined above and described in embodiments herein, both singly and in combination; and X is —C(R)₂-, —NR-, or —O-.

In some embodiments, a compound of Formula VII is covalently linked, either directly or through a linker such as L¹, to at least one structure shown in FIG. 24 or 33 by any chemically feasible means.

In another aspect, the present invention provides a compound of Formula VIII:

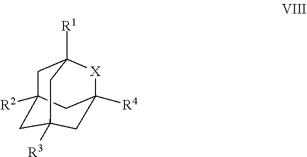

or a pharmaceutically acceptable salt thereof, wherein each of X, R, R¹, R², R₃, R⁶, L¹, L², and L³ is as defined above and described in embodiments herein, both singly and in combination; and R⁴ is independently —R, halogen, —CN, —OR, —N(R)₂, —NO₂, —N₃, —SR, or —L³—R⁶.

In some embodiments, R⁴ is R. In some embodiments, R⁴ is halogen. In some embodiments, R⁴ is —CN. In some embodiments, R⁴ is —OR. In some embodiments, R⁴ is —N(R)₂. In some embodiments, R⁴ is —NO₂. In some embodiments, R⁴ is —N₃. In some embodiments, R⁴ is —SR. In some embodiments, R⁴ is —L³—R⁶.

In some embodiments, R⁴ is hydrogen. In some embodiments, R⁴ is an optionally substituted C₁₋₆ aliphatic group. In some embodiments, R⁴ is an optionally substituted 3-8 membered saturated or partially unsaturated monocyclic carbocyclic ring. In some embodiments, R⁴ is an optionally substituted phenyl. In some embodiments, R⁴ is an optionally substituted 8-10 membered bicyclic aromatic carbocyclic ring. In some embodiments, R⁴ is an optionally substituted 4-8 membered saturated or partially unsaturated monocyclic heterocyclic ring having 1-2 heteroatoms independently selected from nitrogen, oxygen, or sulfur. In some embodiments, R⁴ is an optionally substituted 5-6 membered monocyclic heteroaromatic ring having 1-4 heteroatoms independently selected from nitrogen, oxygen, or sulfur. In some embodiments, R⁴ is an optionally substituted 8-10 membered bicyclic heteroaromatic ring having 1-5 heteroatoms independently selected from nitrogen, oxygen, or sulfur.

In some embodiments, R⁴ is selected from R, halogen, —CN, —OR, —N(R)₂, —SR, C₁₋₆ aliphatic, or —L⁴—R⁶, wherein L⁴ is a C₁₋₆ bivalent straight or branched hydrocarbon chain wherein 1, 2, or 3 methylene units of the chain are independently and optionally replaced with —O-, —C(O)-, —N(R)-, —S-, —SO-, —C(S)-, or —Cy-; wherein the C₁₋₆ aliphatic group is optionally substituted with 1, 2, or 3 groups independently selected from halogen, —CN, —N(R)₂, —NO₂, —N₃, ═NR, ═NOR, ═O, ═S, —OR, —SR, —SO₂R, —S(O)R, —R, —Cy—R, —C(O)R, —C(O)OR, —OC(O)R, —C(O)N(R)₂, —(R)NC(O)R, —OC(O)N(R)₂, —(R)NC(O)OR, —N(R)C(O)N(R)₂, —SO₂N(R)₂, —(R)NSO₂R, —C(S)R, or —C(S)OR; and each R is independently hydrogen, —CH₂-phenyl, phenyl, C₁₋₆ alkyl, cyclopropyl, cyclobutyl, cyclopentyl, cyclohexyl, —CH₂F, —CHF₂, —CF₃, —CH₂CHF₂, or —CH₂CF₃; or each R is independently hydrogen or methyl; or R is hydrogen.

In some embodiments, L⁴ is a covalent bond. In some embodiments, L⁴ is a C₁₋₈ bivalent straight or branched hydrocarbon chain. In some embodiments, L⁴ is a C₁₋₈ bivalent straight or branched hydrocarbon chain wherein 1, 2, or 3 methylene units of the chain are independently and optionally replaced with —O-, —C(O)-, —C(O)O-, —OC(O)-, —C(O)N(R)-, —(R)NC(O)-, —OC(O)N(R)-, —(R)NC(O)O-, —N(R)C(O)N(R)-, —S-, —SO-, —SO₂-, —SO₂N(R)-, —(R)NSO₂-, —C(S)-, —C(S)O-, —OC(S)-, —C(S)N(R)-, —(R)NC(S)-, —(R)NC(S)N(R)-, or —Cy-.

In some embodiments, L⁴ is a C₁₋₆ bivalent straight or branched hydrocarbon chain wherein 1, 2, or 3 methylene units of the chain are independently and optionally replaced with —O-, —C(O)-, —N(R)-, —S-, —SO-, —SO₂N(R)-, —(R)NSO₂-, —C(S)-, or —Cy-, and each R is independently hydrogen, —CH₂-phenyl, phenyl, C₁₋₆ alkyl, cyclopropyl, cyclobutyl, cyclopentyl, cyclohexyl, —CH₂F, —CHF₂, —CF₃, —CH₂CHF₂, or —CH₂CF₃; or each R is independently hydrogen or methyl; or R is hydrogen.

In some embodiments, a compound of Formula VIII is covalently linked, either directly or through a linker such as L¹, to at least one structure shown in FIG. 24 or 33 by any chemically feasible means.

In another aspect, the present invention provides a compound of Formula IX:

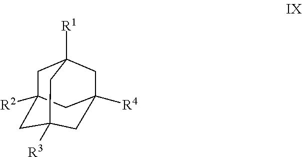

or a pharmaceutically acceptable salt thereof, wherein each of R, R¹, R², R³, R⁴, R⁶, L¹, L², L³, L⁴ and —Cy- is as defined above and described in embodiments herein, both singly and in combination.

In some embodiments, a compound of Formula IX is covalently linked, either directly or through a linker such as L¹, to at least one structure shown in FIG. 24 or 33 by any chemically feasible means.

In some embodiments, the present invention provides a compound of Formula X:

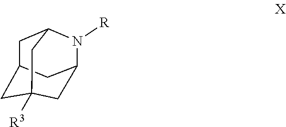

or a pharmaceutically acceptable salt thereof, wherein each of R, R³, R⁶, L³, and —Cy- is as defined above and described in embodiments herein, both singly and in combination.

In some embodiments, a compound of Formula X is covalently linked, either directly or through a linker such as L¹, to at least one structure shown in FIG. 24 or 33 by any chemically feasible means.

In another aspect, the present invention provides a compound of Formula XI:

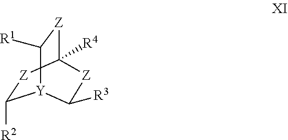

or a pharmaceutically acceptable salt thereof, wherein:
each of Y, R, R¹, R², R³, R⁴, R⁶, L¹, L², L³, L⁴ and —Cy- is as defined above and described in embodiments herein, both singly and in combination; and

### Z is —C(R)₂- or —O-.

In some embodiments, a compound of Formula XI is covalently linked, either directly or through a linker such as L¹, to at least one structure shown in FIG. 24 or 33 by any chemically feasible means.

In some embodiments, the present invention provides a compound of Formula XII:

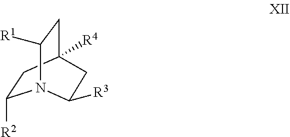

or a pharmaceutically acceptable salt thereof, wherein:
each of R, R¹, R², R³, R⁴, R⁶, L¹, L², L³, L⁴ and —Cy- is as defined above and described in embodiments herein, both singly and in combination.

In some embodiments, a compound of Formula XII is covalently linked, either directly or through a linker such as L¹, to at least one structure shown in FIG. 24 or 33 by any chemically feasible means.

In some embodiments, the present invention provides a compound of Formula XIII:

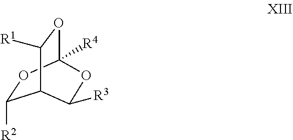

or a pharmaceutically acceptable salt thereof, wherein:
each of R, R¹, R², R³, R⁴, R⁶, L¹, L², L³, L⁴ and —Cy- is as defined above and described in embodiments herein, both singly and in combination.

In some embodiments, a compound of Formula XIII is covalently linked, either directly or through a linker such as L¹, to at least one structure shown in FIG. 24 or 33 by any chemically feasible means.

In another aspect, the present invention provides a compound of Formula XIV:

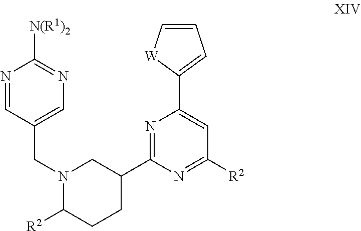

or a pharmaceutically acceptable salt thereof, wherein:
each of R¹, R², R³, R⁶, L¹, L², L³, and —Cy- is as defined above and described in embodiments herein, both singly and in combination; and
W is —NR—, —O-, or —S-.

In some embodiments, a compound of Formula XIV is covalently linked, either directly or through a linker such as L¹, to at least one structure shown in FIG. 24 or 33 by any chemically feasible means.

In another aspect, the present invention provides a compound of Formula XV:

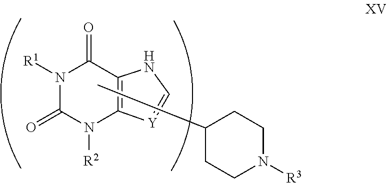

or a pharmaceutically acceptable salt thereof, wherein:
each of Y, R, R¹, R², R³, R⁶, L¹, L², L³, and —Cy- is as defined above and described in embodiments herein, both singly and in combination; except that one of R¹ or R² may be absent and replaced by

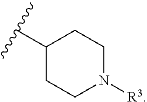

In some embodiments, a compound of Formula XV is covalently linked, either directly or through a linker such as L¹, to at least one structure shown in FIG. 24 or 33 by any chemically feasible means.

In some embodiments, the present invention provides a compound of Formula XVI:

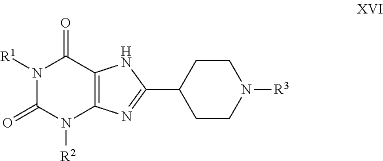

or a pharmaceutically acceptable salt thereof, wherein:
each of R, R¹, R², R³, R⁶, L¹, L², L³, and —Cy- is as defined above and described in embodiments herein, both singly and in combination.

In some embodiments, a compound of Formula XVI is covalently linked, either directly or through a linker such as L¹, to at least one structure shown in FIG. 24 or 33 by any chemically feasible means.

In some embodiments, the present invention provides a compound of Formula XVII:

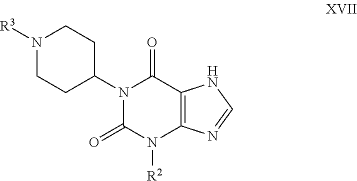

or a pharmaceutically acceptable salt thereof, wherein:
each of R, R², R³, R⁶, L², L³, and —Cy- is as defined above and described in embodiments herein, both singly and in combination.

In some embodiments, a compound of Formula XVII is covalently linked, either directly or through a linker such as L¹, to at least one structure shown in FIG. 24 or 33 by any chemically feasible means.

In some embodiments, the present invention provides a compound of Formula XVIII:

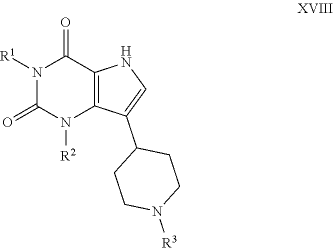

or a pharmaceutically acceptable salt thereof, wherein:
each of R, R¹, R², R³, R⁶, L¹, L², L³, and —Cy- is as defined above and described in embodiments herein, both singly and in combination.

In some embodiments, a compound of Formula XVIII is covalently linked, either directly or through a linker such as L¹, to at least one structure shown in FIG. 24 or 33 by any chemically feasible means.

In some embodiments, the present invention provides a compound of Formula XIX:

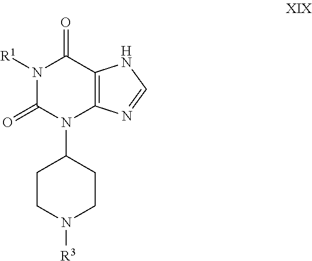

or a pharmaceutically acceptable salt thereof, wherein: each of R, R¹, R³, R⁶, L¹, L³, and —Cy- is as defined above and described in embodiments herein, both singly and in combination.

In some embodiments, a compound of Formula XIX is covalently linked, either directly or through a linker such as L¹, to at least one structure shown in FIG. 24 or 33 by any chemically feasible means.

In some embodiments, the present invention provides a compound of Formula XX:

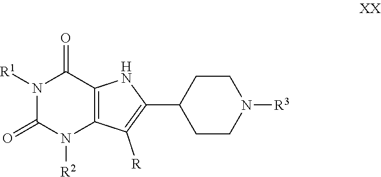

or a pharmaceutically acceptable salt thereof, wherein:
each of R, R¹, R², R³, R⁶, L¹, L², L³, and —Cy- is as defined above and described in embodiments herein, both singly and in combination.

In some embodiments, a compound of Formula XX is covalently linked, either directly or through a linker such as L¹, to at least one structure shown in FIG. 24 or 33 by any chemically feasible means.

In another aspect, the present invention provides a compound of Formula XXI:

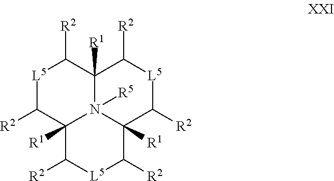

or a pharmaceutically acceptable salt thereof, wherein:
each R, R¹, R², R⁶, L¹, L², and —Cy- is as defined above and described in embodiments herein, both singly and in combination; L⁵ is CH₂ or a single or a double bond; and
R⁵ is absent or is —O⁻.

In some embodiments, a compound of Formula XXI is covalently linked, either directly or through a linker such as L¹, to at least one structure shown in FIG. 24 or 33 by any chemically feasible means.

In some embodiments, the present invention provides a compound of Formula XXII:

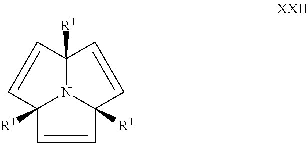

or a pharmaceutically acceptable salt thereof, wherein:
each R, R¹, R⁶, L¹, and —Cy- is as defined above and described in embodiments herein, both singly and in combination.

In some embodiments, a compound of Formula XXII is covalently linked, either directly or through a linker such as L¹, to at least one structure shown in FIG. 24 or 33 by any chemically feasible means.

In some embodiments, the present invention provides a compound of Formula XXIII:

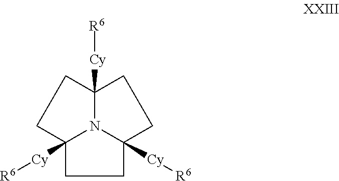

or a pharmaceutically acceptable salt thereof, wherein:
each R, R⁶, and —Cy- is as defined above and described in embodiments herein, both singly and in combination.

In some embodiments, a compound of Formula XXIII is covalently linked, either directly or through a linker such as L¹, to at least one structure shown in FIG. 24 or 33 by any chemically feasible means.

In some embodiments, the present invention provides a compound of Formula XXIV:

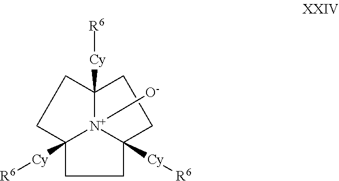

or a pharmaceutically acceptable salt thereof, wherein:
each R, R¹, R⁶, L¹, and —Cy- is as defined above and described in embodiments herein, both singly and in combination.

In some embodiments, a compound of Formula XXIV is covalently linked, either directly or through a linker such as L¹, to at least one structure shown in FIG. 24 or 33 by any chemically feasible means.

In another aspect, the present invention provides a compound of Formula XXV:

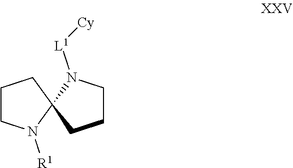

or a pharmaceutically acceptable salt thereof, wherein:
each R, R¹, R⁶, L¹, and —Cy- is as defined above and described in embodiments herein, both singly and in combination.

In some embodiments, a compound of Formula XXV is covalently linked, either directly or through a linker such as L¹, to at least one structure shown in FIG. 24 or 33 by any chemically feasible means.

In some embodiments, the present invention provides a compound of Formulae XXVI-a, XXVI-b, XXVI-c, XXVI-d, or XXVI-e:

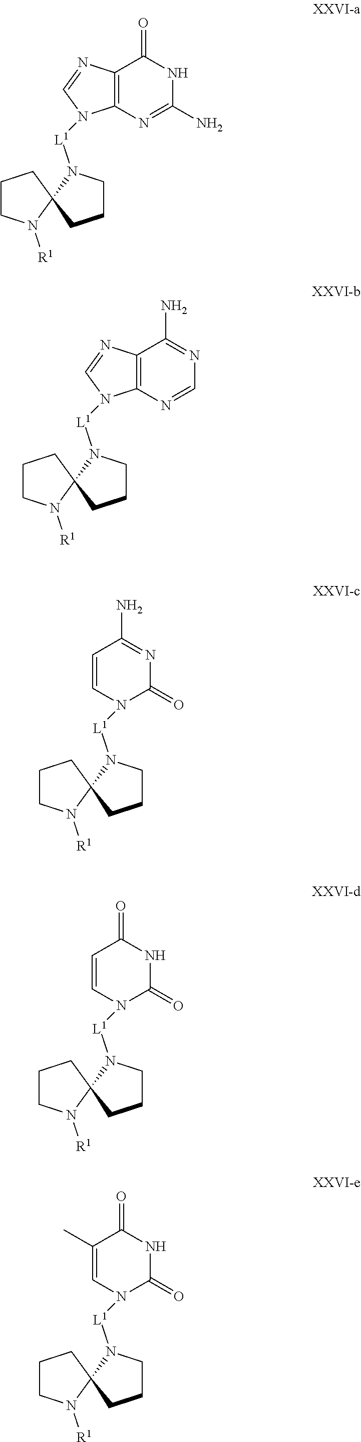

or a diastereomer, or pharmaceutically acceptable salt thereof, wherein:
each R¹, R⁶, L¹, and —Cy- is as defined above and described in embodiments herein, both singly and in combination.

In some embodiments, a compound of Formulae XXVI-a, XXVI-b, XXVI-c, XXVI-d, or XXVI-e is covalently linked, either directly or through a linker such as L¹, to at least one structure shown in FIG. 24 or 33 by any chemically feasible means.

In another aspect, the present invention provides a compound of Formula XXVII:

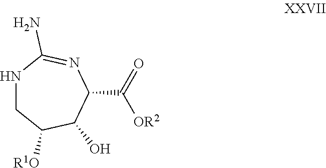

or a pharmaceutically acceptable salt thereof, wherein:
each R, R¹, R², R⁶, L¹, L², and —Cy- is as defined above and described in embodiments herein, both singly and in combination.

In some embodiments, a compound of Formula XXVII is covalently linked, either directly or through a linker such as L¹, to at least one structure shown in FIG. 24 or 33 by any chemically feasible means.

In another aspect, the present invention provides a compound of Formula XXVIII:

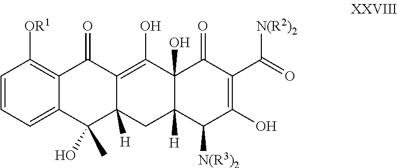

or a pharmaceutically acceptable salt thereof, wherein:
each R, R¹, R², R³, R⁶, L¹, L², L³ and —Cy- is as defined above and described in embodiments herein, both singly and in combination.

In some embodiments, a compound of Formula XXVIII is covalently linked, either directly or through a linker such as L¹, to at least one structure shown in FIG. 24 or 33 by any chemically feasible means.

In another aspect, the present invention provides a compound of Formula XXIX:

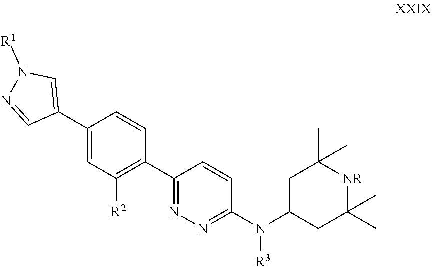

or a pharmaceutically acceptable salt thereof, wherein:
each R, R¹, R², R³, R⁶, L¹, L², L³ and —Cy- is as defined above and described in embodiments herein, both singly and in combination.

In some embodiments, a compound of Formula XXIX is covalently linked, either directly or through a linker such as L¹, to at least one structure shown in FIG. 24 or 33 by any chemically feasible means.

In another aspect, the present invention provides a compound of Formula XXX:

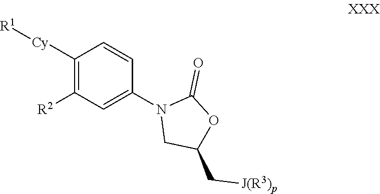

or a pharmaceutically acceptable salt thereof, wherein:
each R, R¹, R², R³, R⁶, L¹, L², L³ and —Cy- is as defined above and described in embodiments herein, both singly and in combination; and

### J is N, O or C, and p is 1, 2, or 3.

In some embodiments, a compound of Formula XXX is covalently linked, either directly or through a linker such as L¹, to at least one structure shown in FIG. 24 or 33 by any chemically feasible means.

In some embodiments, the present invention provides a compound of Formula XXXI:

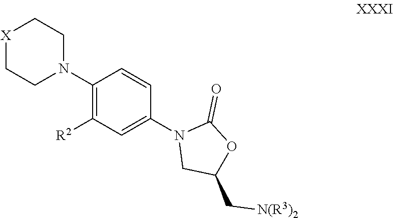

or a pharmaceutically acceptable salt thereof, wherein:
each X, R, R², R³, R⁶, L¹, L², L³ and —Cy- is as defined above and described in embodiments herein, both singly and in combination.

In some embodiments, a compound of Formula XXXI is covalently linked, either directly or through a linker such as L¹, to at least one least one structure shown in FIG. 24 or 33 by any chemically feasible means.

In some embodiments, the present invention provides a compound of Formula XXXII:

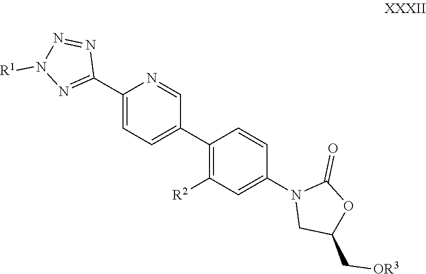

or a pharmaceutically acceptable salt thereof, wherein:
each R, R², R³, R⁶, L¹, L², L³ and —Cy- is as defined above and described in embodiments herein, both singly and in combination.

In some embodiments, a compound of Formula XXXII is covalently linked, either directly or through a linker such as L¹, to at least one least one structure shown in FIG. 24 or 33 by any chemically feasible means.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

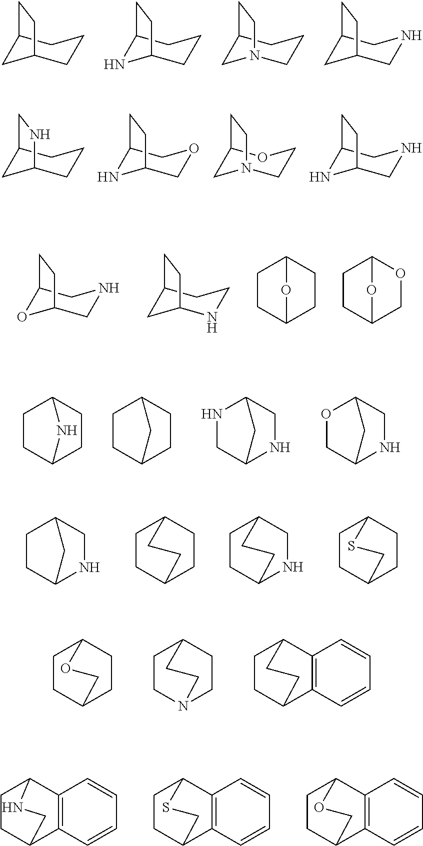

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quatemized form of any basic nitrogen or; a substitutable nitrogen of a heterocyclic ring, for example N (as in 3,4-dihydro-2H-pyrrolyl), NH (as in pyrrolidinyl) or N⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ-, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 □ electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3 -b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R°; —(CH₂)₀₋₄OR°; —O(CH₂)₀₋₄R°, —O—(CH₂)₀₋₄C(O)OR°; —(CH₂)₀₋₄CH(OR°)₂; —(CH₂)₀₋₄R°; —(CH₂)₀₋₄Ph, which may be substituted with R°; —(CH₂)₀₋₄O(CH₂)₀₋₁Ph which may be substituted with R°; —CH═CHPh, which may be substituted with R°; —(CH₂)₀₋₄O(CH₂)₀₋₁-pyridyl which may be substituted with R°; —NO₂; —CN; —N₃; —(CH₂)₀₋₄N(R°)₂; —(CH₂)₀₋₄N(R°)C(O)R°; —N(R°)C(S)R°; —CH₂)₀₋₄N(R°)C(O)NR°₂; —N(R°)C(S)NR°₂; —(CH₂)₀₋₄N(R°)C(O)OR°; —N(R°)N(R°)C(O)R°; —N(R°)N(R°)C(O)NR°₂; —N(R°)N(R°)C(O)OR°; —(CH₂)₀₋₄C(O)OR°; —C(S)R°; —(CH₂)₀₋₄C(O)OR°; —(CH₂)₀₋₄C(O)SR°; —(CH₂)₀₋₄C(O)OSiR°₃; —(CH₂)₀₋₄OC(O)R°; —OC(O)(CH₂)₀₋₄SR-, SC(S)SR°; —(CH₂)₀₋₄SC(O)R°; —(CH₂)₀₋₄C(O)NR°₂; —C(S)NR°₂; —C(S)SR°; —SC(S)SR°, —(CH₂)₀₋₄OC(O)NR°₂; —C(O)N(OR°)R°; —C(O)C(O)R°; —C(O)CH₂C(O)R°; —C(NOR°)R°; —(CH₂)₀₋₄SSR°; —(CH₂)₀₋₄S(O)₂R°; —(CH₂)₀₋₄S(O)₂OR°; —(CH₂)₀₋₄OS(O)₂R°; —S(O)₂NR°₂; —(CH₂)₀₋₄S(O)R°; —N(R°)S(O)₂NR°₂; —N(R°)S(O)₂R°; —N(OR° R°; —C(NH)NR°₂; —P(O)₂R°; —P(O)R°₂, —OP(O)R°₂; —OP(O)(OR°)₂; SiR°₃; —(C₁₋₄ straight or branched)alkylene)O—N(R°)₂; or —(C₁₋₄ straight or branched)alkylene)C(O)O—N(R°)₂, wherein each R° may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R°, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R° (or the ring formed by taking two independent occurrences of R° together with their intervening atoms), are independently halogen, —(CH₂)₀₋₂R^{●}) -(haloR^{●}), —(CH₂)₀₋₂OH, —(CH₂)₀₋₂OR^{●, —(CH}₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R° include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O-, or —S(C(R*₂))₂₋₃S-, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O-, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each Rt is independently hydrogen, C₁-₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, —(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977,66,1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention.

Such compounds are useful, for example, as analytical tools, as probes in biological assays, or as therapeutic agents in accordance with the present invention. In certain embodiments, a warhead moiety, R′, of a provided compound comprises one or more deuterium atoms.

As used herein, the term “inhibitor” is defined as a compound that binds to and/or modulates or inhibits a target RNA with measurable affinity. In certain embodiments, an inhibitor has an IC₅₀ and/or binding constant of less than about 100 μM, less than about 50 μM, less than about 1 μM, less than about 500 nM, less than about 100 nM, less than about 10 nM, or less than about 1 nM.

The terms “measurable affinity” and “measurably inhibit,” as used herein, mean a measurable change in a downstream biological effect between a sample comprising a compound of the present invention, or composition thereof, and a target RNA, and an equivalent sample comprising the target RNA, in the absence of said compound, or composition thereof

The term “RNA” (ribonucleic acid) as used herein, means naturally-occurring or synthetic oligoribonucleotides independent of source (e.g., the RNA may be produced by a human, animal, plant, virus, or bacterium, or may be synthetic in origin), biological context (e.g., the RNA may be in the nucleus, circulating in the blood, in vitro, cell lysate, or isolated or pure form), or physical form (e.g., the RNA may be in single-, double-, or triple-stranded form (including RNA-DNA hybrids), may include epigenetic modifications, native post-transcriptional modifications, artificial modifications (e.g., obtained by chemical or in vitro modification), or other modifications, may be bound to, e.g., metal ions, small molecules, proteins such as chaperones, or co-factors, or may be in a denatured, partially denatured, or folded state including any native or unnatural secondary or tertiary structure such as quadruplexes, hairpins, triplexes, three way junctions (3WJs), four way junctions (4WJs), parallel-Y junctions, hairpins, bulge loops, pseudoknots, and internal loops, etc., and any transient forms or structures adopted by the RNA). In some embodiments, the RNA is 20, 22, 50, 75, or 100 or more nucleotides in length. In some embodiments, the RNA is 250 or more nucleotides in length. In some embodiments, the RNA is 350, 450, 500, 600, 750, or 1,000, 2,000, 3,000, 4,000, 5,000, 7,500, 10,000, 15,000, 25,000, 50,000, or more nucleotides in length. In some embodiments, the RNA is between 250 and 1,000 nucleotides in length. In some embodiments, the RNA is a pre-RNA, pre-miRNA, or pretranscript. In some embodiments, the RNA is a non-coding RNA (ncRNA), messenger RNA (mRNA), micro-RNA (miRNA), a ribozyme, riboswitch, lncRNA, lincRNA, snoRNA, snRNA, scaRNA, piRNA, ceRNA, pseudo-gene, viral RNA, fungal RNA, parasitic RNA, or bacterial RNA. The term “target nucleic acid” or “target RNA,” as used herein, means any type of nucleic acid or RNA, respectively, having a secondary or tertiary structure capable of binding a small molecule compound described herein. In some embodiments, the structure is a 3WJ that is bound to or stabilized by a disclosed compound or a 3WJ that is stabilized by the binding of the compound at another site on the target nucleic acid (e.g. RNA). All 3WJ structures are encompassed by the present invention without reference to their conformation or attendant tertiary interactions. For example, the 3WJ may be a cis 3WJ, a trans 3WJ, a parallel-Y junction, or another form of 3WJ. The target RNA may be inside a cell, in a cell lysate, or in isolated form prior to contacting the compound.

The term “effector” or “effector nucleic acid,” as used herein, means a nucleic acid that binds to a target nucleic acid and regulates or modulates its activity. Examplary effectors include small RNAs acting in trans to induce 3WJ formation. In some embodiments, effectors are selected from various natural forms of small RNA such as miRNA, Piwi-interacting RNA (piRNA), and small nucleolar RNA (snoRNA). These small RNAs can base pair with a target such as mRNA in a manner that results in the formation of a 3WJ. The base pairing between the effector and target is often incomplete, meaning that the two sequences have some, but not complete, complementarity. Perfect complementarity would result in the formation of a fully double-stranded structure that lacks a 3WJ. In order to form a 3WJ, the effector (e.g., a miRNA) and target (e.g., an mRNA) would generally form a stretch of base pairing of at least 4 nucleotides (nt) followed by a base-pairing stem of at least 4 nt and a loop of unpaired nt, followed by a second stretch of base pairing between the effector and target sequences. The stem-loop can be formed either in the effector or the target RNA.

The term “cis 3WJ” or “cis three-way junction,” as used herein, refers to a 3WJ formed between portions of a single nucleic acid, such as a single strand of mRNA, precursor, or protein-bound complex thereof.

The term “trans 3WJ” or “trans three-way junction,” as used herein, refers to a 3WJ formed between two or more nucleic acids, such as an miRNA/mRNA complex sharing partial sequence complementarity.

### 4. General Methods of Providing the Present Compounds

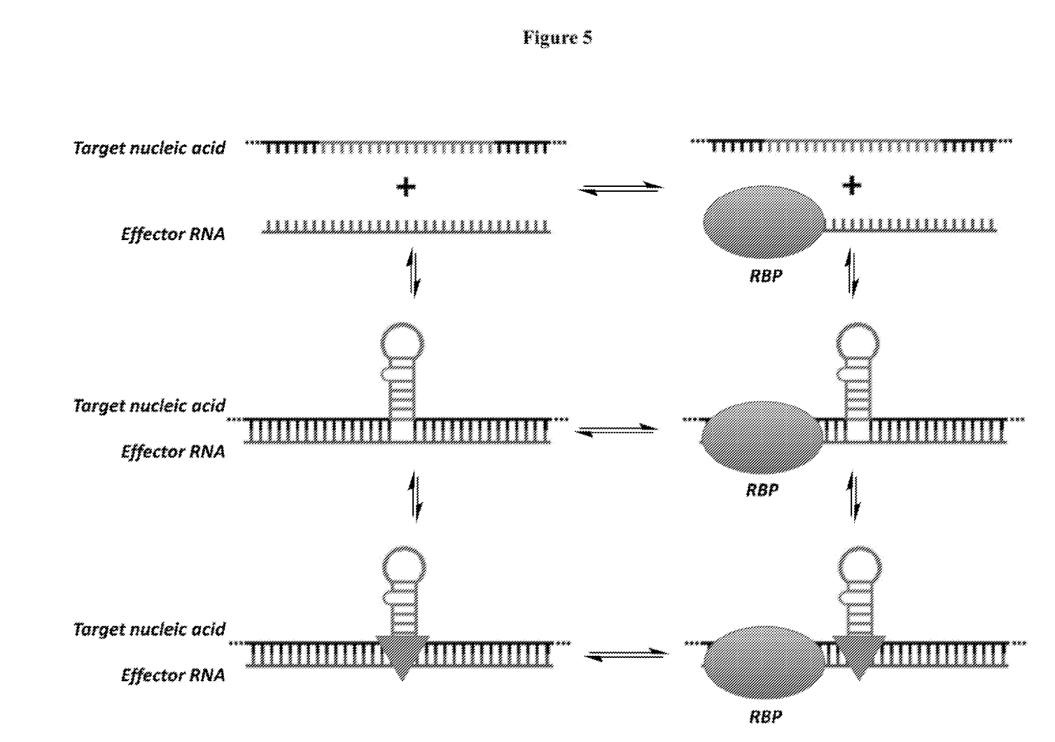

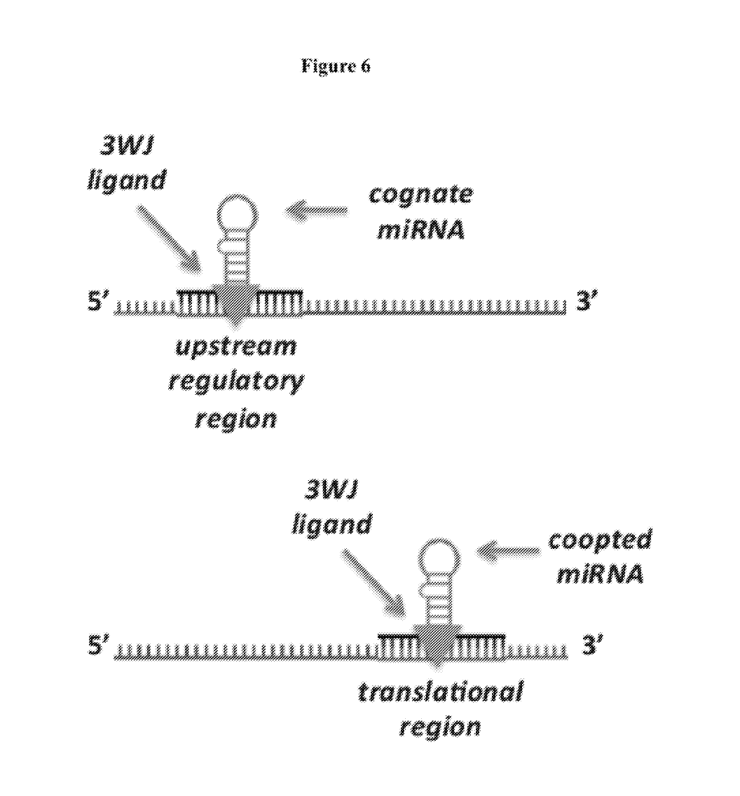

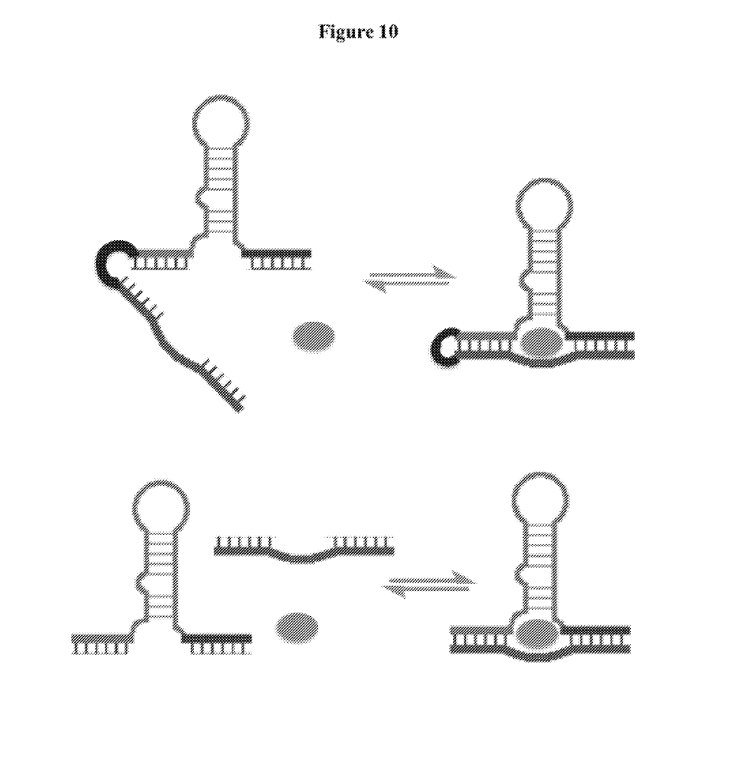

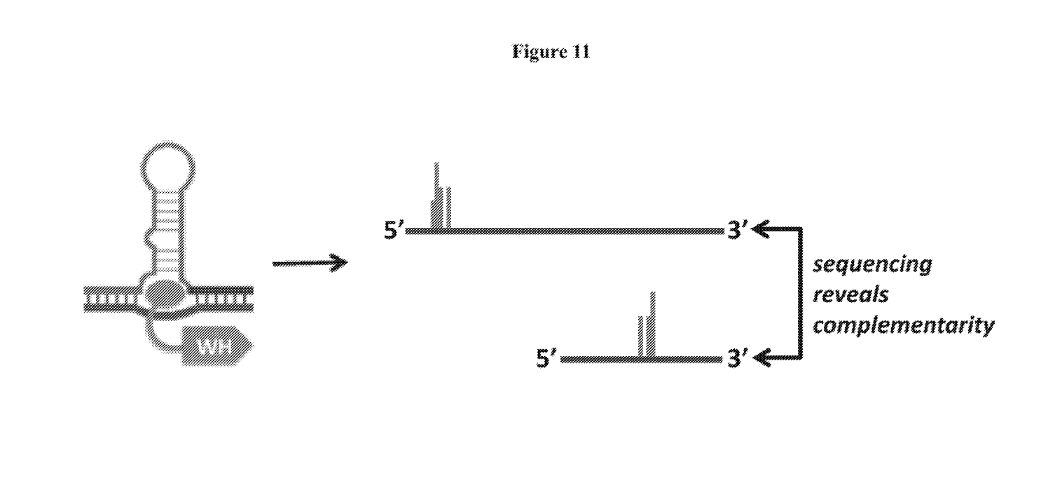

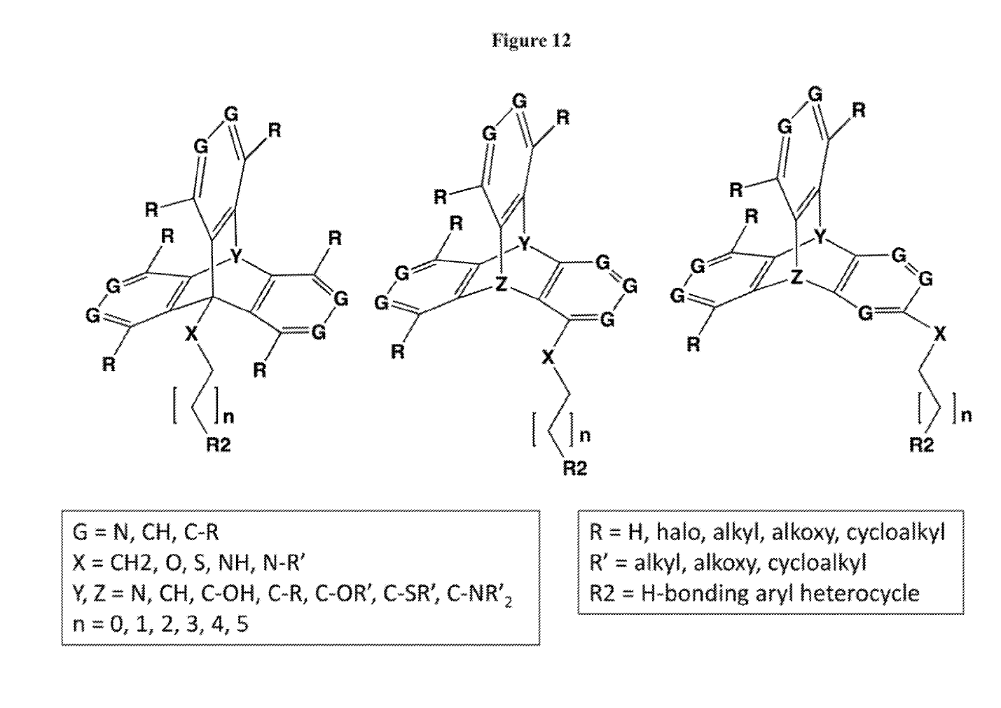

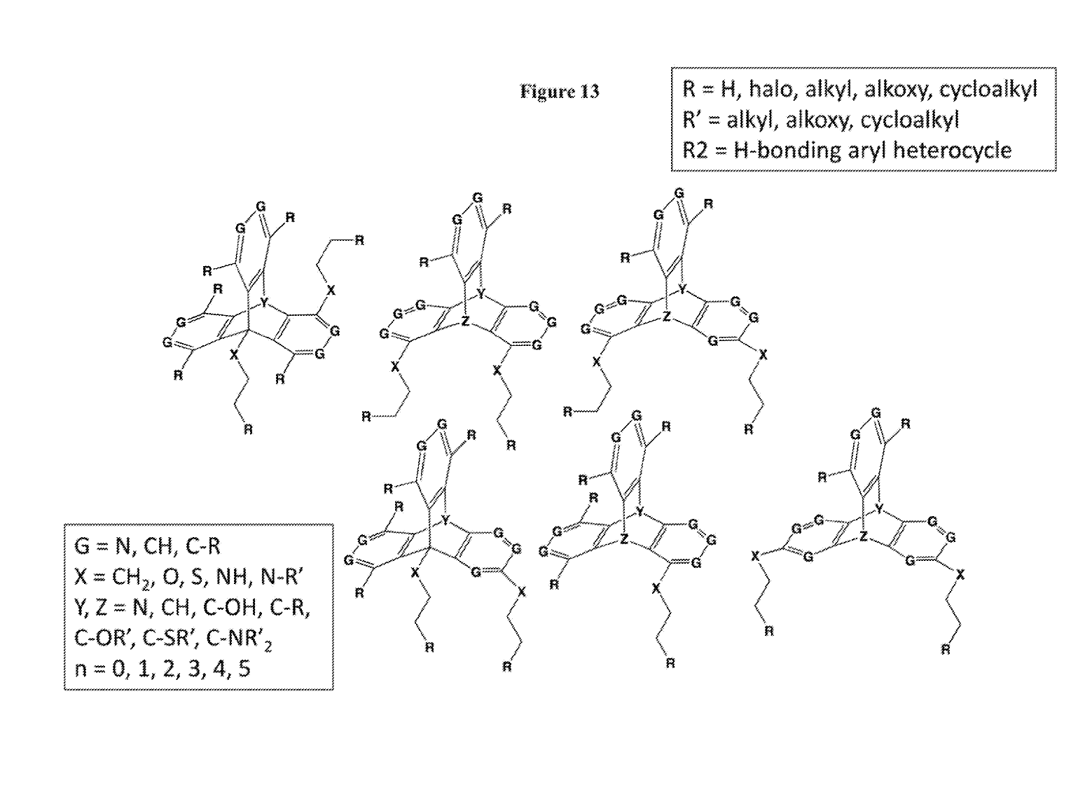

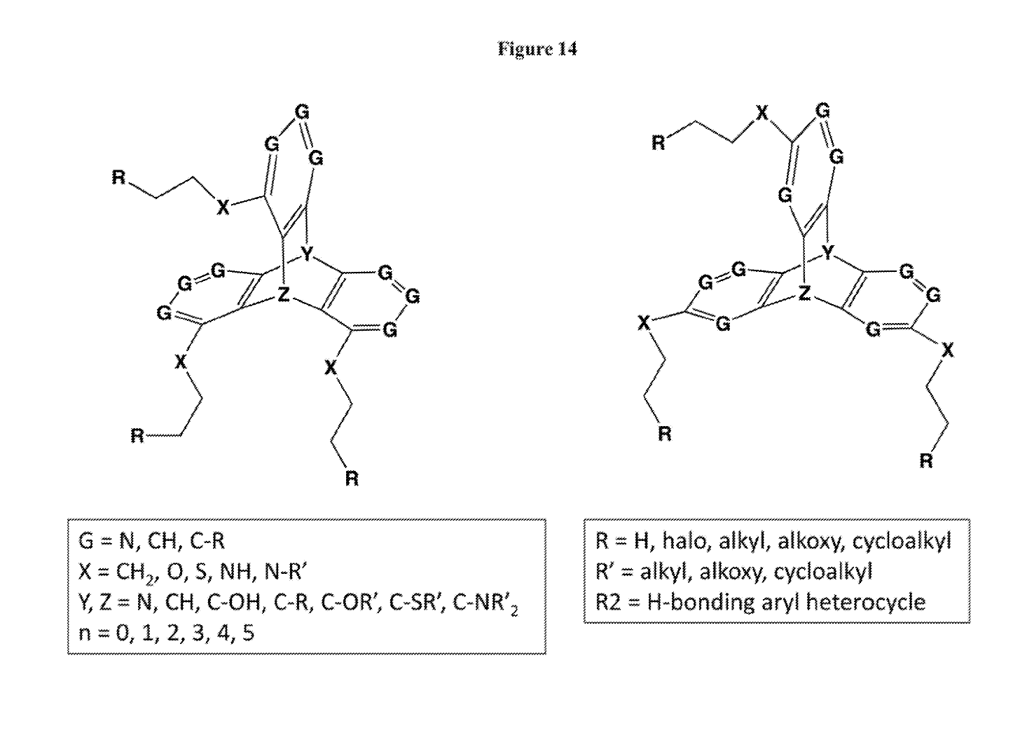

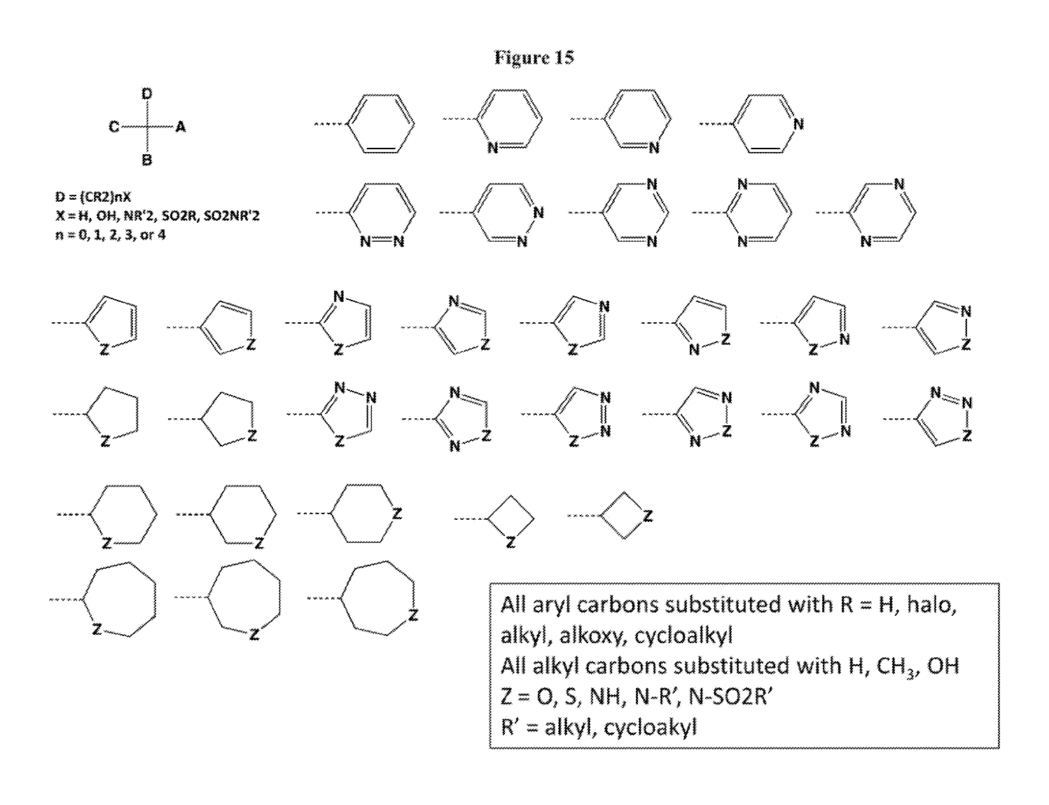

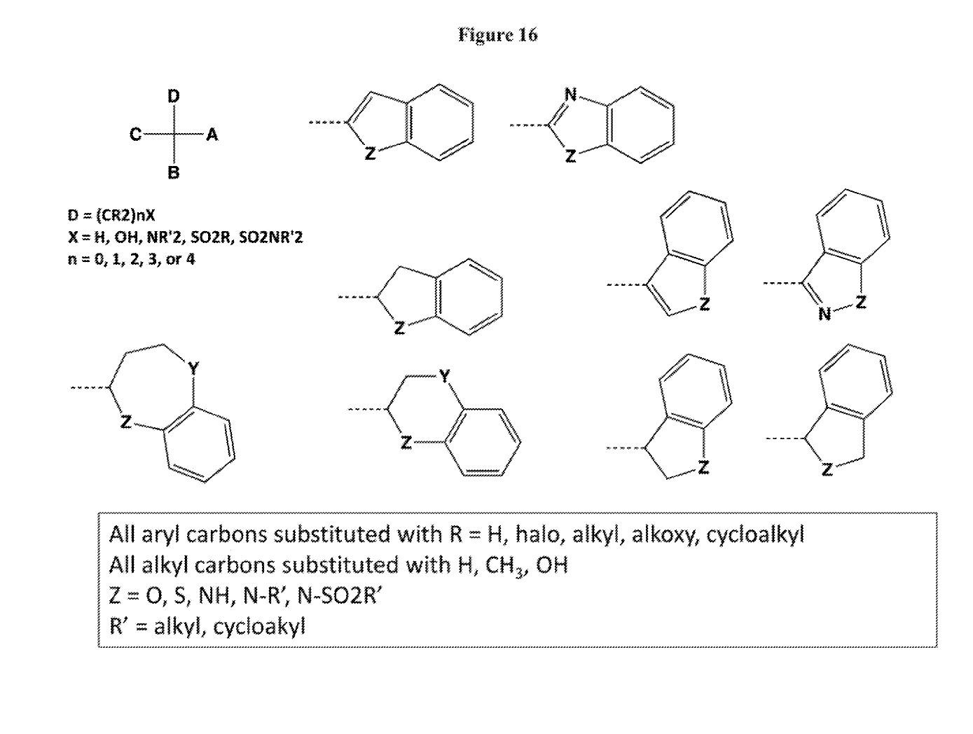

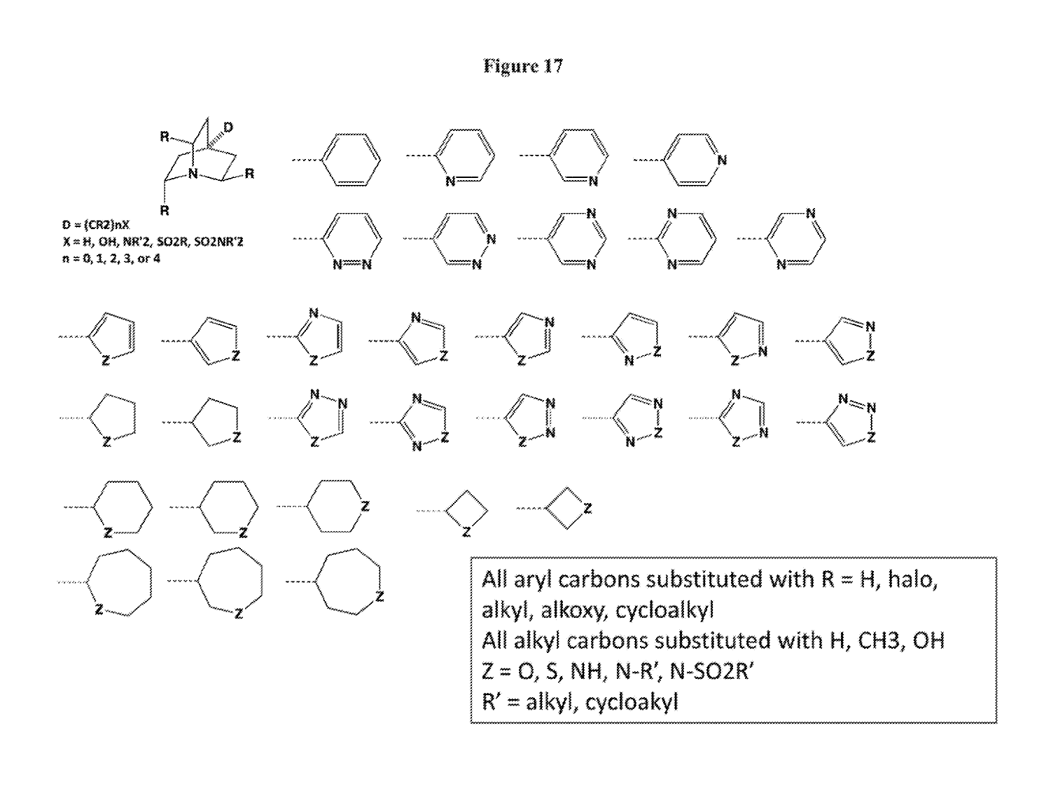

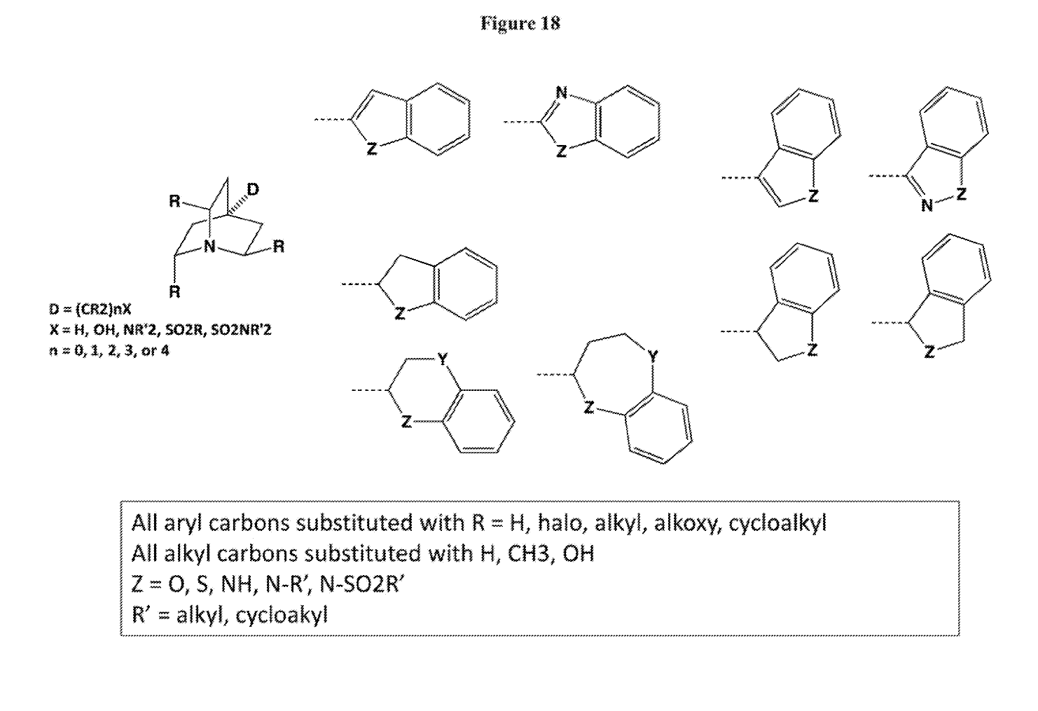

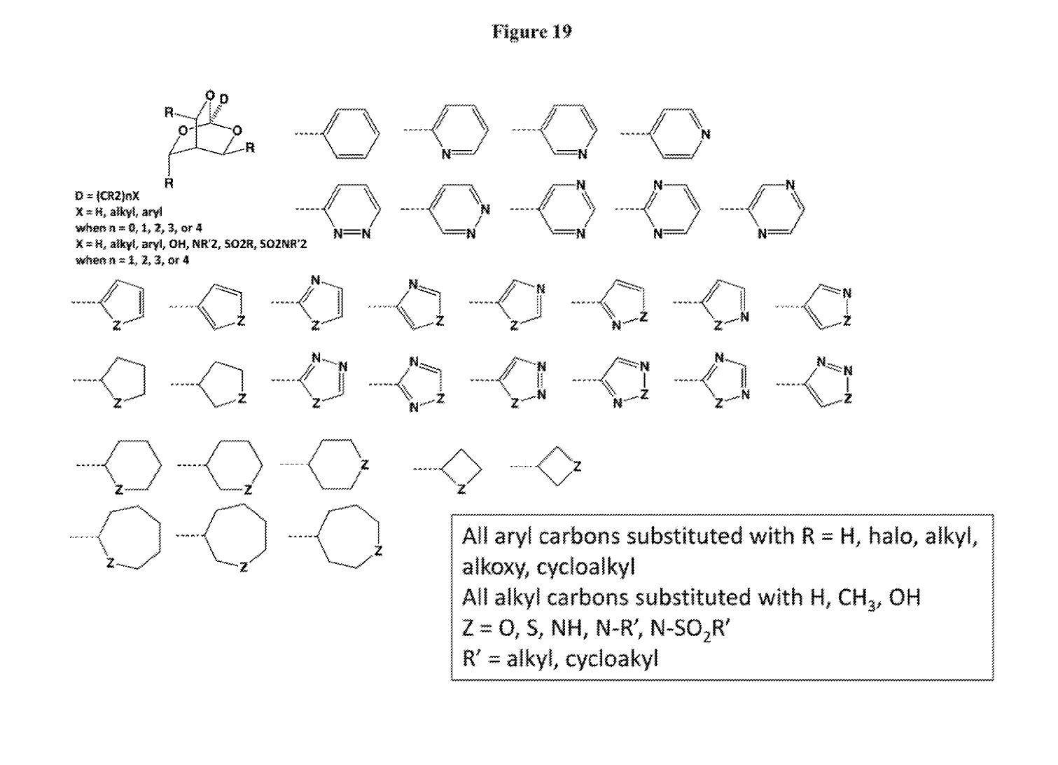

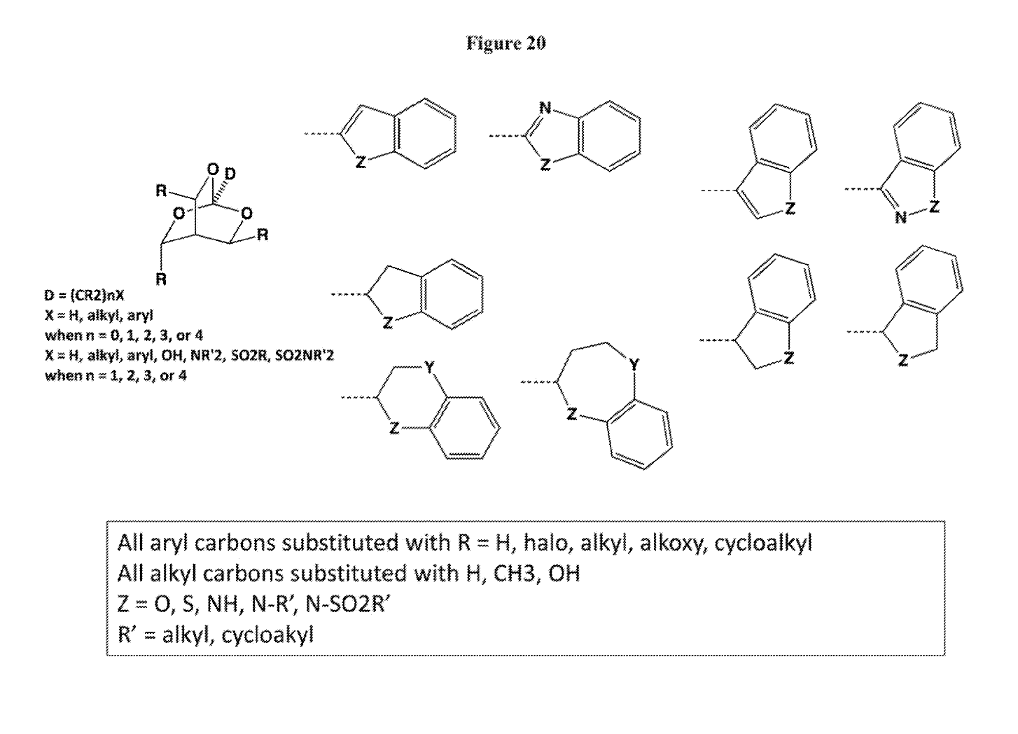

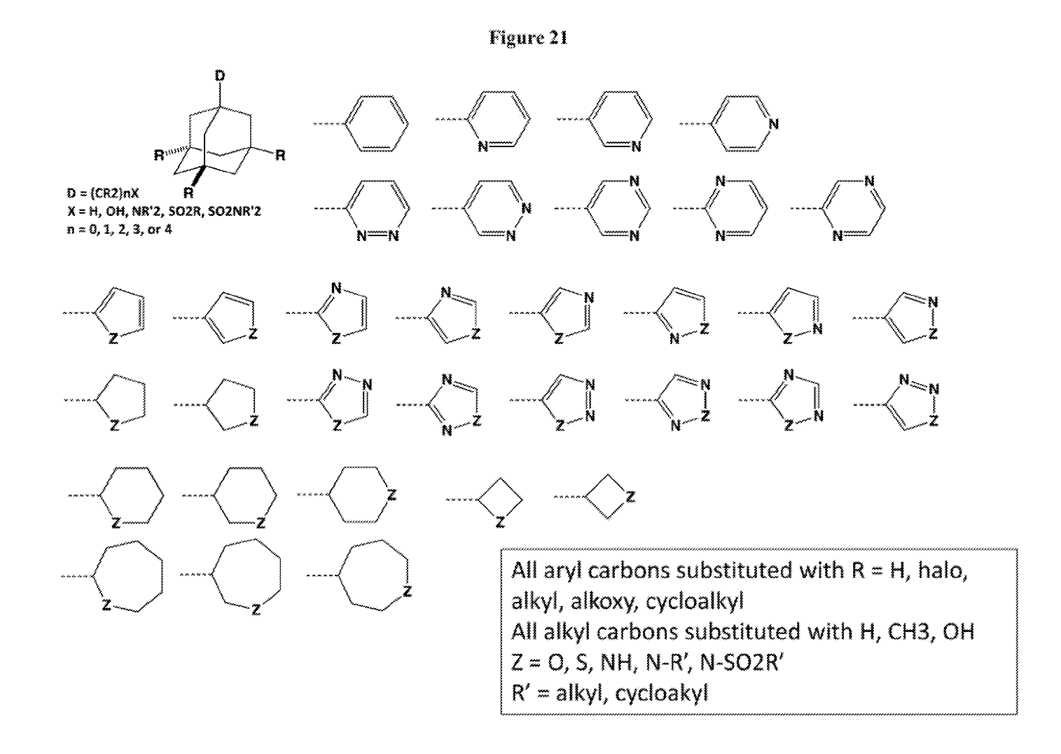

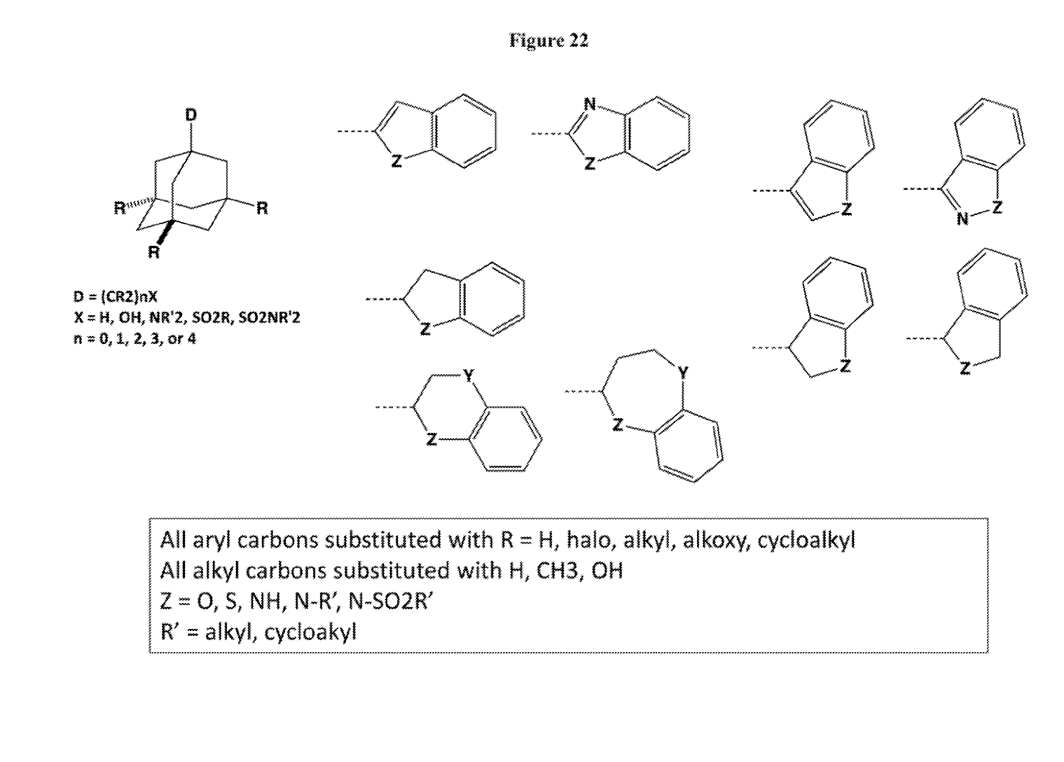

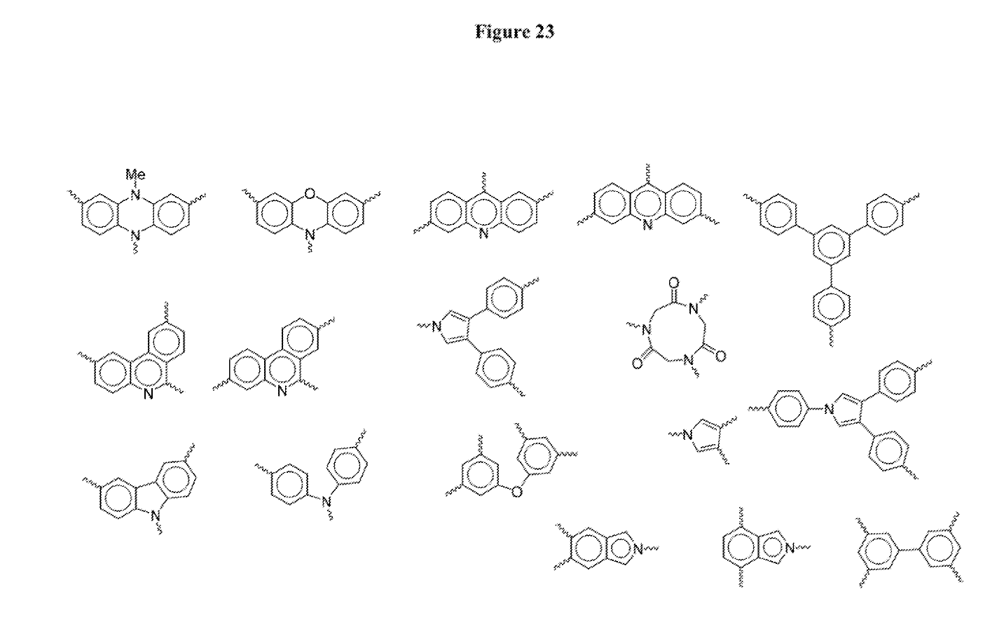

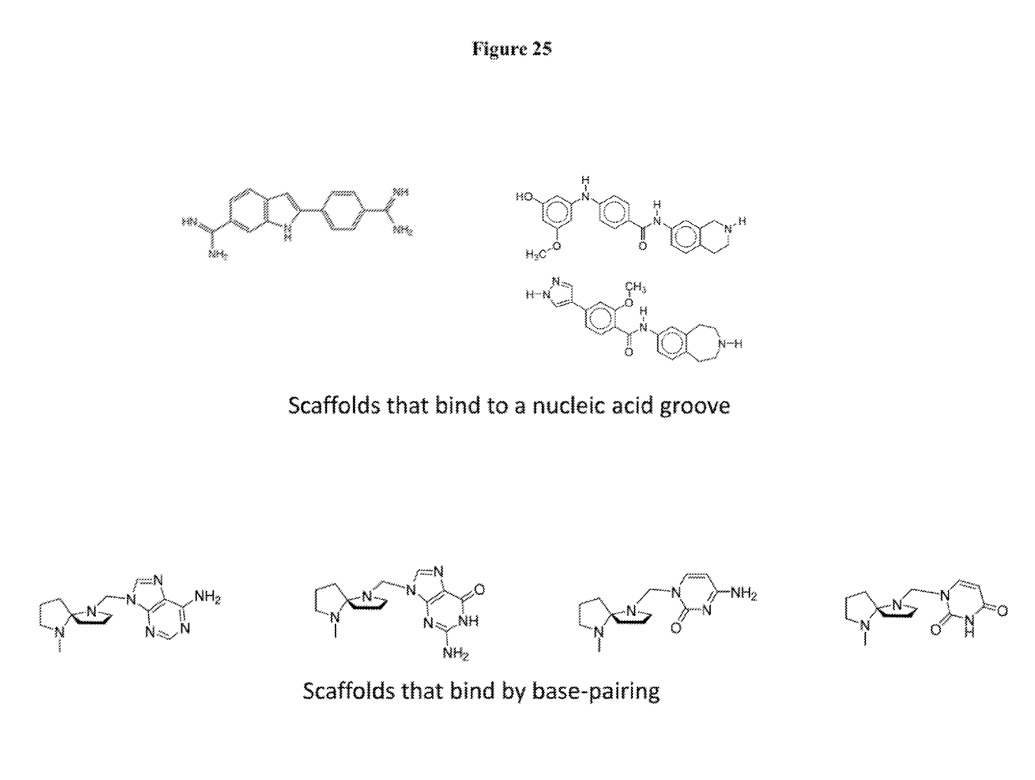

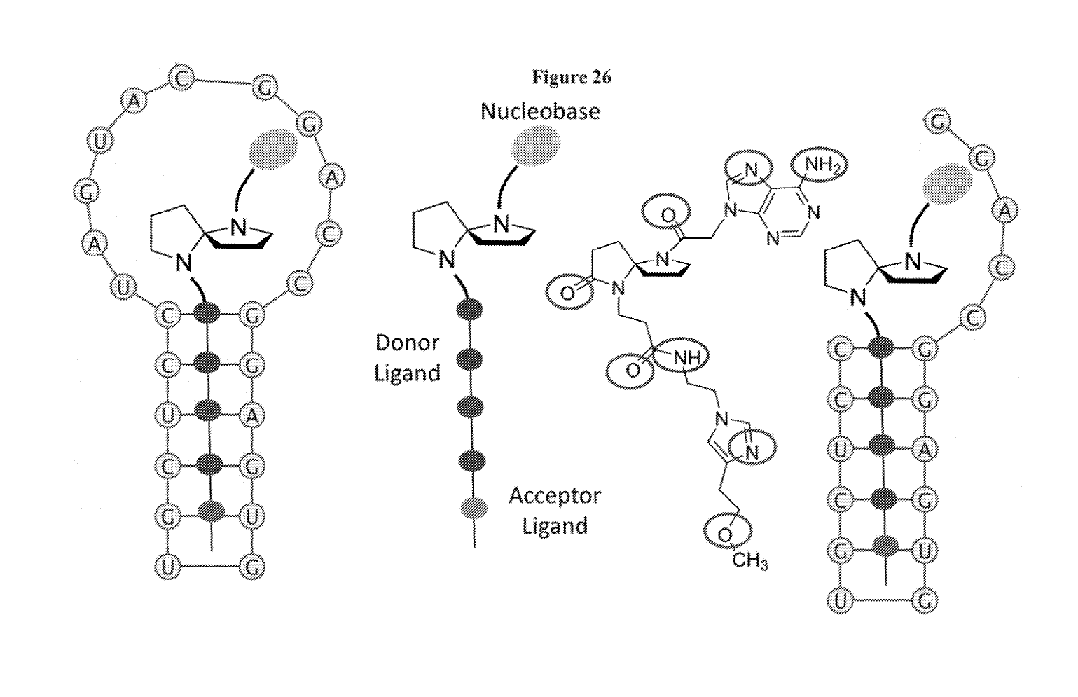

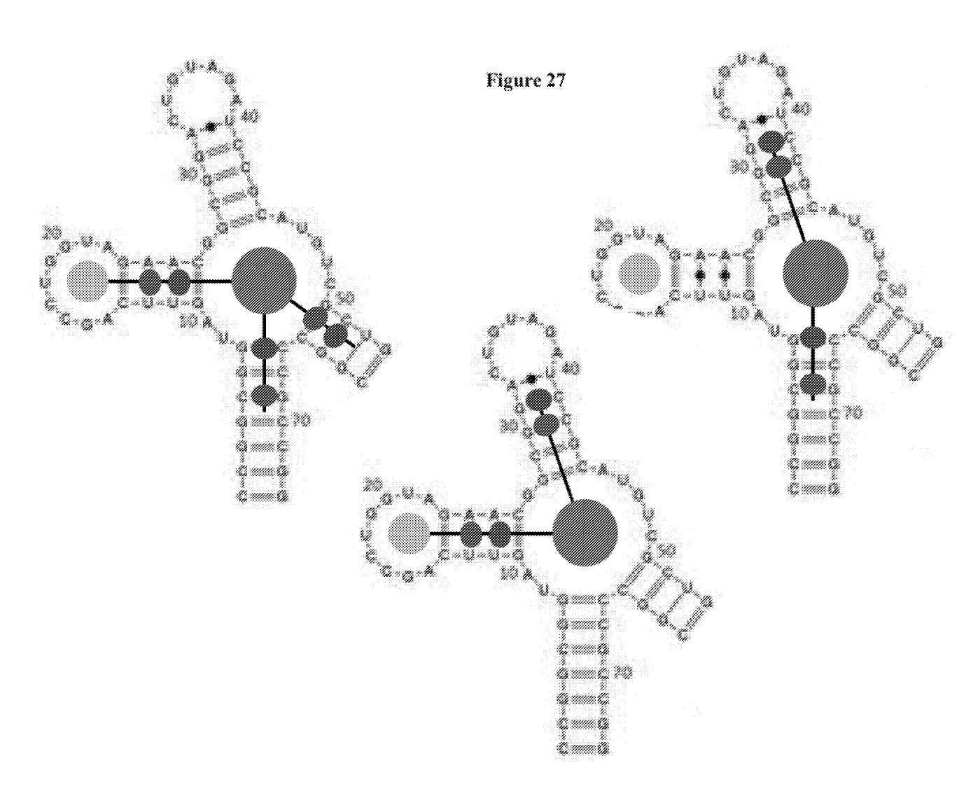

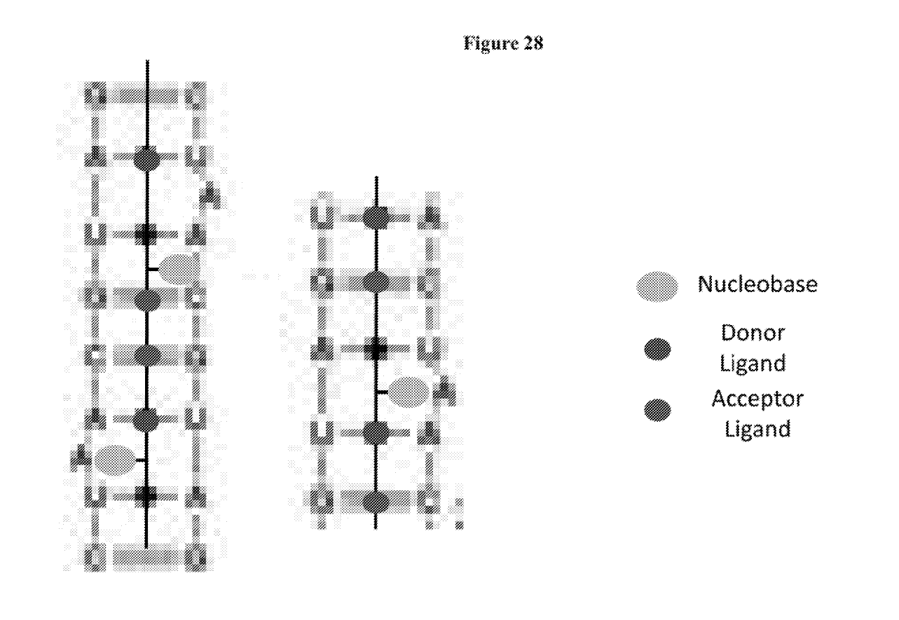

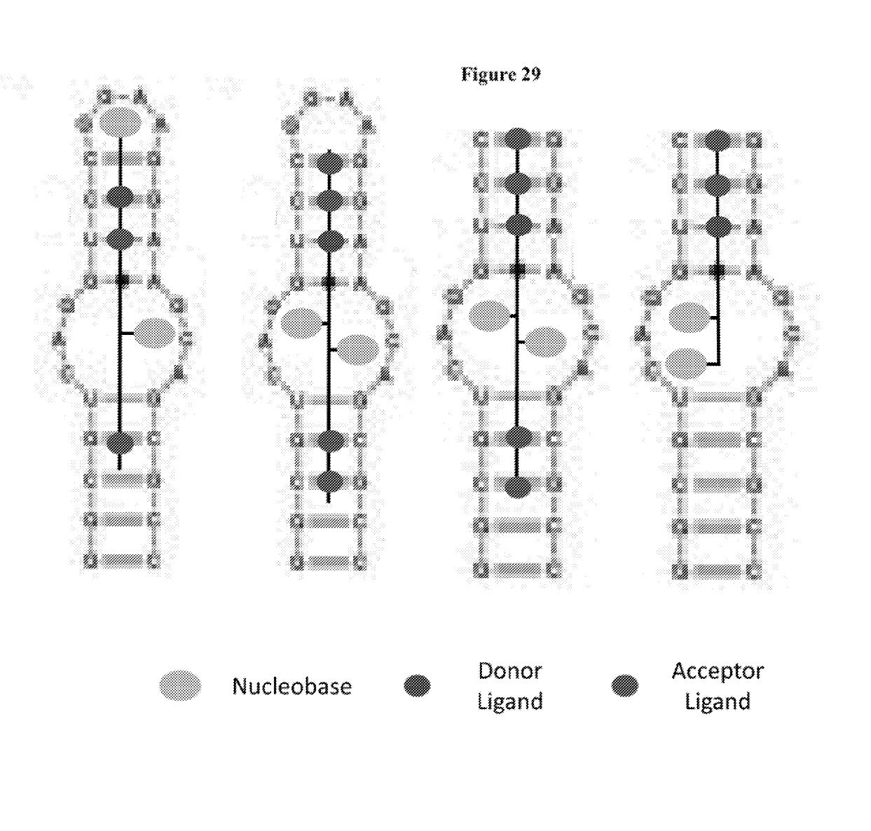

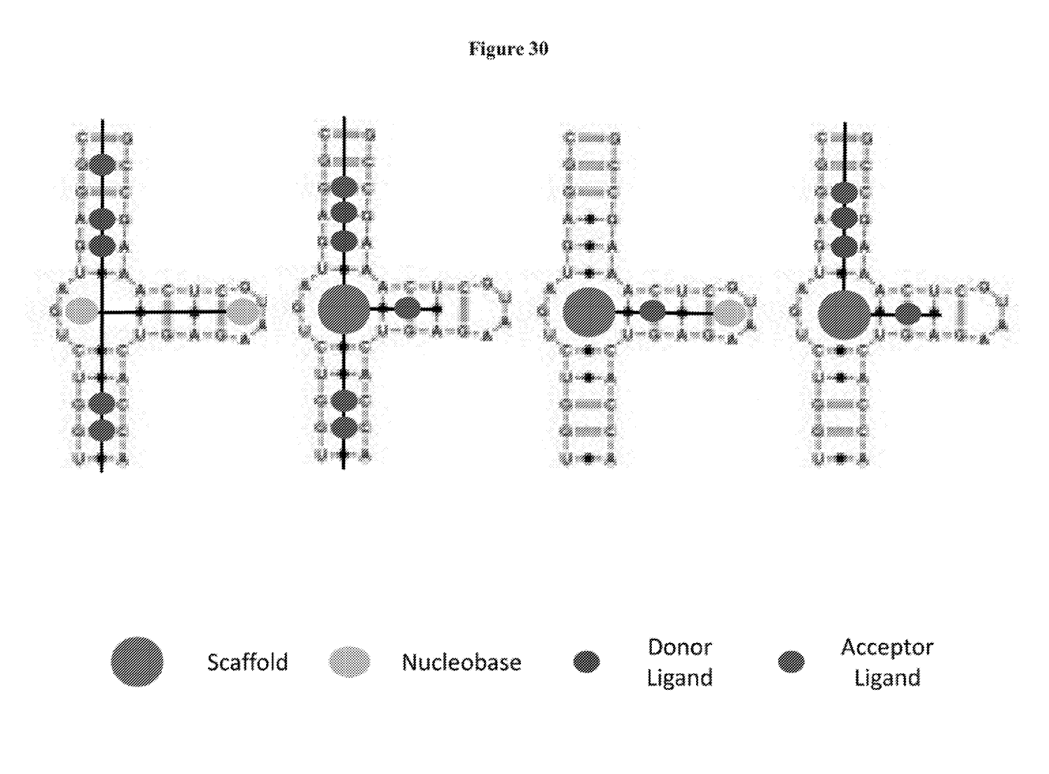

The compounds of this invention may be prepared or isolated in general by synthetic and/or semi-synthetic methods known to those skilled in the art for analogous compounds and by methods described in detail in the Examples and Figures, herein. For example, various compounds of the present invention may be synthesized by reference to FIGS. 5-31 or 77-94 or 96 of U.S. Provisional Patent Application U.S. Ser. No. 62/289,671, which is hereby incorporated by reference in its entirety.

In the schemes and chemical reactions depicted in the detailed description, Examples, and Figures, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “leaving group” (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethyl silyl, t-butyl dimethylsilyl, t-butyldiphenyl silyl, trii sopropyl silyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halob enzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3′ edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5th Ed., Ed.: Smith, M.B. and March, J., John Wiley & Sons, New York: 2001, the entirety of which is incorporated herein by reference. Such interconversions may require one or more of the aforementioned techniques, and certain methods for synthesizing compounds of the invention are described below in the Exemplification and Figures.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably inhibit or modulate a target RNA, or a mutant thereof, in a biological sample or in a patient. In certain embodiments, the amount of compound in compositions of this invention is such that is effective to measurably inhibit or modulate a target RNA, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the modulation of a target RNA to retreat an RNA-mediated disease or condition.

The activity of a compound utilized in this invention to modulate a target RNA may be assayed in vitro, in vivo or in a cell line. In vitro assays include assays that determine modulation of the target RNA. Alternate in vitro assays quantitate the ability of the compound to bind to the target RNA. Detailed conditions for assaying a compound utilized in this invention to modulate a target RNA are set forth in the Examples below.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are modulators of a target RNA and are therefore useful for treating one or more disorders associated with or affected by (e.g., downstream of) the target RNA. Thus, in certain embodiments, the present invention provides a method for treating an RNA-mediated disorder comprising the step of administering to a patient in need thereof a compound of the present invention, or pharmaceutically acceptable composition thereof

As used herein, the terms “RNA-mediated” disorders, diseases, and/or conditions as used herein means any disease or other deleterious condition in which RNA, such as an overexpressed, underexpressed, mutant, misfolded, pathogenic, or ongogenic RNA, is known to play a role. Accordingly, another embodiment of the present invention relates to treating or lessening the severity of one or more diseases in which RNA, such as an overexpressed, underexpressed, mutant, misfolded, pathogenic, or ongogenic RNA, is known to play a role.

In some embodiments, the present invention provides a method for treating one or more disorders, diseases, and/or conditions wherein the disorder, disease, or condition includes, but is not limited to, a cellular proliferative disorder.

### Cellular Proliferative Disorders

The present invention features methods and compositions for the diagnosis and prognosis of cellular proliferative disorders (e.g., cancer) and the treatment of these disorders by modulating a target RNA. Cellular proliferative disorders described herein include, e.g., cancer, obesity, and proliferation-dependent diseases. Such disorders may be diagnosed using methods known in the art.

### Cancer

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). In some embodiments, the cancer is melanoma or breast cancer.

Cancers includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the present invention provides a method for treating a tumor in a patient in need thereof, comprising administering to the patient any of the compounds, salts or pharmaceutical compositions described herein. In some embodiments, the tumor comprises any of the cancers described herein. In some embodiments, the tumor comprises melanoma cancer. In some embodiments, the tumor comprises breast cancer. In some embodiments, the tumor comprises lung cancer. In some embodiments the the tumor comprises small cell lung cancer (SCLC). In some embodiments the the tumor comprises non-small cell lung cancer (NSCLC).

In some embodiments, the tumor is treated by arresting further growth of the tumor. In some embodiments, the tumor is treated by reducing the size (e.g., volume or mass) of the tumor by at least 5%, 10%, 25%, 50%, 75%, 90% or 99% relative to the size of the tumor prior to treatment. In some embodiments, tumors are treated by reducing the quantity of the tumors in the patient by at least 5%, 10%, 25%, 50%, 75%, 90% or 99% relative to the quantity of tumors prior to treatment.

### Other Proliferative Diseases

Other proliferative diseases include, e.g., obesity, benign prostatic hyperplasia, psoriasis, abnormal keratinization, lymphoproliferative disorders (e.g., a disorder in which there is abnormal proliferation of cells of the lymphatic system), chronic rheumatoid arthritis, arteriosclerosis, restenosis, and diabetic retinopathy. Proliferative diseases that are hereby incorporated by reference include those described in U.S. Pat. Nos. 5,639,600 and 7,087,648.

### Inflammatory Disorders and Diseases

Compounds of the invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease which can be treated according to the methods of this invention is an disease of the skin. In some embodiments, the inflammatory disease of the skin is selected from contact dermatitits, atompic dermatitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, and other inflammatory or allergic conditions of the skin.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, Systemic jubenile idiopathic arthritis (SJIA), Cryopyrin Associated Periodic Syndrome (CAPS), and osteoarthritis.

In some embodiments the inflammatory disease which can be treated according to the methods of this invention is a TH17 mediated disease. In some embodiments the TH17 mediated disease is selected from Systemic lupus erythematosus, Multiple sclerosis, and inflammatory bowel disease (including Crohn's disease or ulcerative colitis).

In some embodiments the inflammatory disease which can be treated according to the methods of this invention is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis. Metabolic Disease

In some embodiments the invention provides a method of treating a metabolic disease. In some embodiments the metabolic disease is selected from Type 1 diabetes, Type 2 diabetes, metabolic syndrome or obesity.

The compounds and compositions, according to the method of the present invention, may be administered using any amount and any route of administration effective for treating or lessening the severity of a cancer, an autoimmune disorder, a proliferative disorder, an inflammatory disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of modulating the activity of a target RNA in a biological sample comprising the step of contacting said biological sample with a compound of this invention, or a composition comprising said compound.

According to another embodiment, the invention relates to a method of modulating the activity of a target RNA in a biological sample comprising the step of contacting said biological sample with a compound of this invention, or a composition comprising said compound. In certain embodiments, the invention relates to a method of irreversibly inhibiting the activity of a target RNA in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Another embodiment of the present invention relates to a method of modulating the activity of a target RNA in a patient comprising the step of administering to said patient a compound of the present invention, or a composition comprising said compound.

According to another embodiment, the invention relates to a method of inhibiting the activity of a target RNA in a patient comprising the step of administering to said patient a compound of the present invention, or a composition comprising said compound. According to certain embodiments, the invention relates to a method of irreversibly inhibiting the activity of a target RNA in a patient comprising the step of administering to said patient a compound of the present invention, or a composition comprising said compound. In other embodiments, the present invention provides a method for treating a disorder mediated by a target RNA in a patient in need thereof, comprising the step of administering to said patient a compound according to the present invention or pharmaceutically acceptable composition thereof. Such disorders are described in detail herein.

### EXEMPLIFICATION

As depicted in the Examples below, in certain exemplary embodiments, compounds are prepared according to the following general procedures and used in biological assays and other procedures described generally herein. It will be appreciated that, although the general methods depict the synthesis of certain compounds of the present invention, the following general methods, and other methods known to one of ordinary skill in the art, can be applied to all compounds and subclasses and species of each of these compounds, as described herein. Similarly, assays and other analyses can be adapted according to the knowledge of one of ordinary skill in the art.

### EXAMPLE 1

Historical efforts to identify small molecule ligands that bind to RNA have focused on base-pairing or on canonical structural motifs in duplex RNA: intercalation between bases and/or groove binding. But these motifs do not support selective binding of small molecules to specific RNAs. However, RNA folds into an enormous variety of complex tertiary structures that present pockets conducive to small molecule binding—small molecules that are complementary to the shape and electrostatics presented by those pockets. Insofar as the details of shape and electrostatics reflect the underlying sequence of the RNA, small molecules can achieve selectivity, much as they do when binding protein pockets.

Indeed, there are now several reports of drug-like small molecules that bind to RNA, many of them FDA-approved (see Table 4 below).

### Small Molecule Ligands by Class

Though a range of small molecule chemotypes has been demonstrated to bind to folded RNA (Guan & Disney, ACS Chem. Biol. 2012 7, 73-86), hereby incorporated by reference, there are limited reports of high-throughput screening of large libraries (>10⁵ compounds) to identify RNA-binding ligands. Accordingly there are also few reports of small molecules synthetically optimized for RNA binding. The present invention paves the path to a remedy for these deficiencies. Below is a table summarizing the broad chemotypes which have demonstrable RNA binding and will serve as the starting point to optimize and validate our screening method, which will in turn enable the systematic screening of essentially all known chemotypes against RNA structures of therapeutic interest.

TABLE 4
RNA-binding Small Molecules

| Small Molecule | States | RNA Target | Reference |
|---|---|---|---|
| Linezolid | FDA-approved | Bacterial | Leach et al. |
| | antibiotic | ribosomal RNA | Mol. Cell 2007, |
| | | | 26, 393-402 |
| Tedizolid | FDA-approved | Bacterial | Leach et al. |
| | antibiotic | ribosomal RNA | Mol. Cell 2007, |
| | | | 26, 393-402 |
| Tetracycline | FDA-approved | Bacterial 30S | Brodersen et al. |
| | antibiotic | ribosomal RNA | Cell 2000, |
| | | | 103, 1143-1154 |
| Amino- | FDA-approved | Bacterial 16S | Fourmy et al. |
| glycosides | antibiotics | ribosomal RNA | Science 1996, |
| | | | 274, 1367-1371 |
| Theophylline | FDA-approved | Aptameric RNA | Jenison et al. |
| | for COPD | | Science 1994, |
| | and asthma | | 263, 1425-1429 |

These discoveries revealed a molecular mechanism of action that was not anticipated. The intentional design of small molecules that bind to folded RNA has been pursued only rarely because of substantial technical challenges, with one notable example being the design of triptycene-based ligands able to bind selectively to RNA three-way junctions (Barros et al., Angew. Chem. Int. Ed. 2014, 53, 13746-13750. Triptycene-based ligands will thus provide another chemotype with RNA binding ability to serve as another starting point in the described screening methods.

The Figures provide many exemplary scaffolds and specific target compound genera, which may be combined or altered using ordinary experimentation.

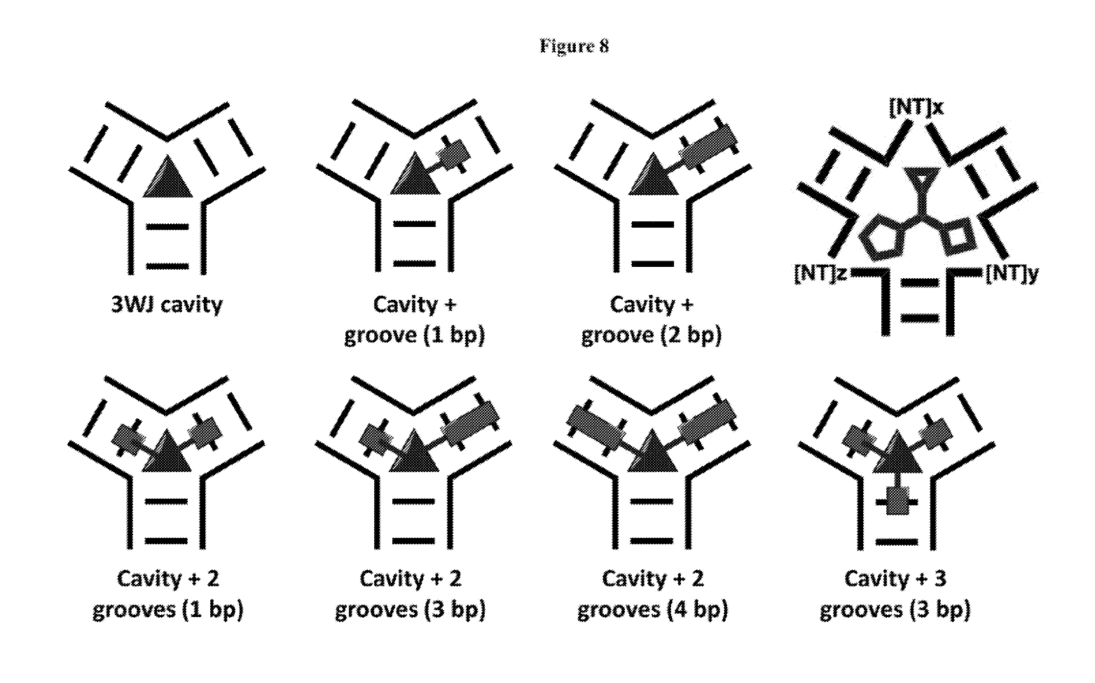

As shown in FIG. 8 (top right structure), while tripartite scaffolds represent a promising platform for binding 3WJs, they do not need to be symmetric, as the 3WJ will itself not usually be symmetric. In the pictured example, the triangle, square, and pentagon stand for cyclopropyl, cyclobutyl, or cyclopentyl, and may be substituted with the various nucleic acid binding groups disclosed herein.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound as shown or described in any one of the Formulae I-XXXII above or in any one of FIGSsssss. 1-67.
